SEQUENCE LISTING (100 sequences)

SEQ ID NO: 1 - DNA, 306 bp, Homo sapiens
tcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcag

SEQ ID NO: 2 - DNA, 126 bp, Mus musculus
atggagtccgctgcagacagactggccagggcggcggcccagggccgtgtgcatgacgtgcgggcactgctggaagccggggtttcgcccaacgccccgaactctttcggtcgtaccccgattcag

SEQ ID NO: 3 - DNA, 318 bp, Mus musculus
tgatgatgatgggcaacgttcacgtagcagctcttctgctcaactacggtgcagattcgaactgcgaggaccccactaccttctcccgcccggtgcacgacgcagcgcgggaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatag

SEQ ID NO: 4 - DNA, 22 bp, Homo sapiens
ggcggagctgctgctgctccac

SEQ ID NO: 5 - DNA, 22 bp, Homo sapiens
gcccgtggacctggctgaggag

SEQ ID NO: 6 - DNA, 22 bp, Homo sapiens
ggcagtaaccatgcccgcatag

SEQ ID NO: 7 - DNA, 22 bp, Homo sapiens
cagtaaccatgcccgcatagat

SEQ ID NO: 8 - DNA, 22 bp, Homo sapiens
gcccgcatagatgccgcggaag

SEQ ID NO: 9 - DNA, 63 bp, Homo sapiens
agcggagctgctgctgctccactagtgaagccacagatgtagtggagcagcagcagctccgcc

SEQ ID NO: 10 - DNA, 63 bp, Homo sapiens
acccgtggacctggctgaggagtagtgaagccacagatgtactcctcagccaggtccacgggc

SEQ ID NO: 11 - DNA, 63 bp, Homo sapiens
agcagtaaccatgcccgcatagtagtgaagccacagatgtactatgcgggcatggttactgcc

SEQ ID NO: 12 - DNA, 63 bp, Homo sapiens
aagtaaccatgcccgcatagattagtgaagccacagatgtaatctatgcgggcatggttactg

SEQ ID NO: 13 - DNA, 63 bp, Homo sapiens
acccgcatagatgccgcggaagtagtgaagccacagatgtacttccgcggcatctatgcgggc

SEQ ID NO: 14 - DNA, 46 bp, Mus musculus
ctctggctttcgtgaacatgtcgaaacatgttcacgaaagccagag

SEQ ID NO: 15 - DNA, 46 bp, Mus musculus
gctcttctgctcaactacggtcgaaaccgtagttgagcagaagagc

SEQ ID NO: 16 - DNA, 46 bp, Mus musculus
gctcaactacggtgcagattccgaagaatctgcaccgtagttgagc

SEQ ID NO: 17 - DNA, 23 bp, Homo sapiens
ggtaccgtgcgacatcgcgatgg

SEQ ID NO: 18 - DNA, 23 bp, Homo sapiens
tgggccatcgcgatgtcgcacgg

SEQ ID NO: 19 - DNA, 23 bp, Homo sapiens
accttccgcggcatctatgcggg

SEQ ID NO: 20 - DNA, 23 bp, Homo sapiens
tgtcgcacggtacctgcgcgcgg

SEQ ID NO: 21 - DNA, 23 bp, Homo sapiens
ccgcggcatctatgcgggcatgg

SEQ ID NO: 22 - DNA, 23 bp, Homo sapiens
gaccttccgcggcatctatgcgg

SEQ ID NO: 23 - DNA, 23 bp, Homo sapiens
gcccgcatagatgccgcggaagg

SEQ ID NO: 24 - DNA, 23 bp, Homo sapiens
ccatgcccgcatagatgccgcgg

SEQ ID NO: 25 - DNA, 23 bp, Homo sapiens
acggtacctgcgcgcggctgcgg

SEQ ID NO: 26 - DNA, 23 bp, Homo sapiens
tcccgggcagcgtcgtgcacggg

SEQ ID NO: 27 - DNA, 23 bp, Mus musculus
accgaaagagttcggggcgttgg

SEQ ID NO: 28 - DNA, 23 bp, Mus musculus
ggtacgaccgaaagagttcgggg

SEQ ID NO: 29 - DNA, 23 bp, Mus musculus
cggggcgttgggcgaaaccccgg

SEQ ID NO: 30 - DNA, 23 bp, Mus musculus
ccgaaagagttcggggcgttggg

SEQ ID NO: 31 - DNA, 23 bp, Mus musculus
gggccgtgtgcatgacgtgcggg

SEQ ID NO: 32 - DNA, 23 bp, Unknown
  Description of Unknown Mouse or rat sequence
cggtgcagattcgaactgcgagg

SEQ ID NO: 33 - DNA, 23 bp, Unknown
  Description of Unknown Mouse or rat sequence
cccgcgctgcgtcgtgcaccggg

SEQ ID NO: 34 - DNA, 23 bp, Unknown
  Description of Unknown Mouse or rat sequence
cgctgcgtcgtgcaccgggcggg

SEQ ID NO: 35 - DNA, 23 bp, Unknown
  Description of Unknown Mouse or rat sequence
gcgctgcgtcgtgcaccgggcgg

SEQ ID NO: 36 - DNA, 818 bp, Homo sapiens
caaagggcggcgcagcggctgccgagctcggccctggaggcggcgagaacatggtgcgcaggttcttggtgaccctccggattcggcgcgcgtgcggcccgccgcgagtgagggttttcgtggttcacatctcgtggttcacgggggagtgggcagcgccaggggcgcccgccgctgtggccctcgtgctgatgctactgaggagccagcgtctagggcagcagccgcttcctagaagaccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctccggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaaaaaaaaaaaaaaaaaaaa

SEQ ID NO: 37 - DNA, 913 bp, Homo sapiens
tggcgcactcagccaggcccgcgggcatctgctgcgtgtcccgctccgggctcagtgccctcgccgccgccggcactgcctcgatgttccagctgcccatcttgaatttcagcccccagcaagtggccggggtatgtgagaccctggaagagagcggcgatgtggagcgcctgggtcgcttcctctggtcgctgcccgtggcccctgcggcctgcgaggccctcaacaagaatgagtcggtgctacgcgcacgagccatcgtggcctttcacggtggcaactaccgcgagctctatcatatcctggaaaaccacaagttcaccaaggagtcgcacgccaagctgcaggcgctgtggcttgaagcacactaccaggaggctgagaagctgcgtggaagacccctgggacctgtggacaagtaccgagtaaggaagaagttcccgctgccgcgcaccatttgggacggcgaacagaagacacactgcttcaaggagcgcacgcggaacctgctacgcgagtggtacctgcaggatccataccctaaccccagcaaaaaacgtgagctcgcccaggcaaccggactgacccctacgcaggtgggcaactggttcaaaaaccgccgacaaagggaccgagcggctgcagccaagaacagactccagcagcaggtcctgtcacagggttccgggcgggcactacgggcggagggcgacggcacgccagaggtgctgggcgtcgccaccagcccggccgccagtctatccagcaaggcggccacttcagccatctccatcacgtccagcgacagcgagtgcgacatctgagttgcccatccaggatgctcagaagcagattccagtgtaaaaacgagaaaaacaaaatgaaagaggggaagaagatgagagacctgcaa

SEQ ID NO: 38 - DNA, 2508 bp, Homo sapiens [sequence omitted]

SEQ ID NO: 39 - DNA, 1522 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaaaaaaaaaaaaaaaaaaaaaaaaaaa

SEQ ID NO: 40 - DNA, 498 bp, Homo sapiens
atgctgctggaggaggttcgcgccggcgaccggctgagtggggcggcggcccggggcgacgtgcaggaggtgcgccgccttctgcaccgcgagctggtgcatcccgacgccctcaaccgcttcggcaagacggcgctgcaggtcatgatgtttggcagcaccgccatcgccctggagctgctgaagcaaggtgccagccccaatgtccaggacacctccggtaccagtccagtccatgacgcagcccgcactggattcctggacaccctgaaggtcctagtggagcacggggctgatgtcaacgtgcctgatggcaccggggcacttccaatccatctggcagttcaagagggtcacactgctgtggtcagctttctggcagctgaatctgatctccatcgcagggacgccaggggtctcacacccttggagctggcactgcagagaggggctcaggacctcgtggacatcctgcagggccacatggtggccccgctg

SEQ ID NO: 41 - DNA, 31 bp, Artificial Sequence
  Description of Artificial Sequence Synthetic primer
cggtgtaggagctgctggtgcaggggccacg

SEQ ID NO: 42 - DNA, 21 bp, Artificial Sequence
  Description of Artificial Sequence Synthetic primer
gtaagatgtgctgggactact

SEQ ID NO: 43 - DNA, 22 bp, Artificial Sequence
  Description of Artificial Sequence Synthetic primer
cgaactcccgacctcaggtgat

SEQ ID NO: 44 - DNA, 31 bp, Artificial Sequence
  Description of Artificial Sequence Synthetic primer
ctgtggtgtatgttggaataaatatcgaata

SEQ ID NO: 45 - DNA, 20 bp, Artificial Sequence
  Description of Artificial Sequence Synthetic primer
gagtcaacggatttggtcgt

SEQ ID NO: 46 - DNA, 20 bp, Artificial Sequence
  Description of Artificial Sequence Synthetic primer
gacaagcttcccgttctcag

SEQ ID NO: 47 - DNA, 18 bp, Artificial Sequence
  Description of Artificial Sequence Synthetic primer
gagcagcatggagccttc

SEQ ID NO: 48 - DNA, 20 bp, Artificial Sequence
  Description of Artificial Sequence Synthetic primer
cctccgaccgtaactattcg

SEQ ID NO: 49 - DNA, 20 bp, Artificial Sequence
  Description of Artificial Sequence Synthetic primer
agaatgagtcggtgctacgc

SEQ ID NO: 50 - DNA, 21 bp, Artificial Sequence
  Description of Artificial Sequence Synthetic primer
gcctcctggtagttgtgcttc

SEQ ID NO: 51 - DNA, 23 bp, Artificial Sequence
  Description of Artificial Sequence Synthetic primer
actcacctcttcagaacgaattg

SEQ ID NO: 52 - DNA, 23 bp, Artificial Sequence
  Description of Artificial Sequence Synthetic primer
ccatctttggaaggttcaggttg

SEQ ID NO: 53 - DNA, 20 bp, Artificial Sequence
  Description of Artificial Sequence Synthetic primer
gtcaaggccgagaatgggaa

SEQ ID NO: 54 - DNA, 20 bp, Artificial Sequence
  Description of Artificial Sequence Synthetic primer
ttggctccacccttcaagtg

SEQ ID NO: 55 - DNA, 18 bp, Artificial Sequence
  Description of Artificial Sequence Synthetic primer
gcggactccatgctgctc

SEQ ID NO: 56 - DNA, 18 bp, Artificial Sequence
  Description of Artificial Sequence Synthetic primer
cacgactgggcgattggg

SEQ ID NO: 57 - DNA, 20 bp, Artificial Sequence
  Description of Artificial Sequence Synthetic primer
actccagcagcaggttctgt

SEQ ID NO: 58 - DNA, 20 bp, Artificial Sequence
  Description of Artificial Sequence Synthetic primer
agatgtcgcactcactgtcg

SEQ ID NO: 59 - DNA, 20 bp, Artificial Sequence
  Description of Artificial Sequence Synthetic primer
cgcaggttcttggtcactgt

SEQ ID NO: 60 - DNA, 20 bp, Artificial Sequence
  Description of Artificial Sequence Synthetic primer
tgttcacgaaagccagagcg

SEQ ID NO: 61 - DNA, 20 bp, Artificial Sequence
  Description of Artificial Sequence Synthetic primer
ttgcggaaggcggagggaac

SEQ ID NO: 62 - DNA, 20 bp, Artificial Sequence
  Description of Artificial Sequence Synthetic primer
aagagcagggccaccgtgac

SEQ ID NO: 63 - DNA, 22 bp, Artificial Sequence
  Description of Artificial Sequence Synthetic primer
cgatccggagcagcatggagtc

SEQ ID NO: 64 - DNA, 22 bp, Artificial Sequence
  Description of Artificial Sequence Synthetic primer
ttccagcagtgcccgcacctcg

SEQ ID NO: 65 - DNA, 18 bp, Artificial Sequence
  Description of Artificial Sequence Synthetic primer
gccttcttgggactgatg

SEQ ID NO: 66 - DNA, 18 bp, Artificial Sequence
  Description of Artificial Sequence Synthetic primer
tgtgggtggtatcctctg

SEQ ID NO: 67 - DNA, 18 bp, Artificial Sequence
  Description of Artificial Sequence Synthetic primer
caggagtcccatgtaaga

SEQ ID NO: 68 - DNA, 18 bp, Artificial Sequence
  Description of Artificial Sequence Synthetic primer
acagggaaacacttctgc

SEQ ID NO: 69 - DNA, 18 bp, Artificial Sequence
  Description of Artificial Sequence Synthetic primer
accctgtccctcaaatcc

SEQ ID NO: 70 - DNA, 18 bp, Artificial Sequence
  Description of Artificial Sequence Synthetic primer
ggtgccacattcgctaag

SEQ ID NO: 71 - DNA, 18 bp, Artificial Sequence
  Description of Artificial Sequence Synthetic primer
caatatggcggtggaagg

SEQ ID NO: 72 - DNA, 18 bp, Artificial Sequence
  Description of Artificial Sequence Synthetic primer
ccgcaaccaatggatctc

SEQ ID NO: 73 - DNA, 18 bp, Artificial Sequence
  Description of Artificial Sequence Synthetic primer
atggagcccggactacag

SEQ ID NO: 74 - DNA, 18 bp, Artificial Sequence
  Description of Artificial Sequence Synthetic primer
ggtgttagcgtgggtagc

SEQ ID NO: 75 - DNA, 21 bp, Artificial Sequence
  Description of Artificial Sequence Synthetic primer
cactgtgacaagcgaggtgag

SEQ ID NO: 76 - DNA, 21 bp, Artificial Sequence
  Description of Artificial Sequence Synthetic primer
gatgggcgtggagcaaagatg

SEQ ID NO: 77 - DNA, 19 bp, Artificial Sequence
  Description of Artificial Sequence Synthetic primer
aagttgtgcctctgcactc

SEQ ID NO: 78 - DNA, 19 bp, Artificial Sequence
  Description of Artificial Sequence Synthetic primer
gcgattgatgcctccaaag

SEQ ID NO: 79 - protein, 6 aa, Artificial Sequence
  Description of Artificial Sequence Synthetic 6xHis tag
HHHHHH

SEQ ID NO: 80 - DNA, 22 bp, Artificial Sequence
  Description of Artificial Sequence Synthetic primer
gtggcctttcacggtggcaact

SEQ ID NO: 81 - DNA, 21 bp, Artificial Sequence
  Description of Artificial Sequence Synthetic primer
gttgcccacctgcgtaggggt

SEQ ID NO: 82 - DNA, 42 bp, Artificial Sequence
  Description of Artificial Sequence Synthetic primer
ggttagggtatggatcctgcaggtaccactcgcgtagcaggt

SEQ ID NO: 83 - DNA, 21 bp, Artificial Sequence
  Description of Artificial Sequence Synthetic primer
gatgctgtccccggacgatat

SEQ ID NO: 84 - DNA, 21 bp, Artificial Sequence
  Description of Artificial Sequence Synthetic primer
gcccagacggaaaccgtagct

SEQ ID NO: 85 - protein, 29 aa, Homo sapiens
DGEQKTHCFKERTRHLLREWYLQDPYPNP

SEQ ID NO: 86 - protein, 29 aa, Pan troglodytes
DGEQKTHCFKERTRHLLREWYLQDPYPNP

SEQ ID NO: 87 - protein, 29 aa, Macaca mulatta
DGEQKTHCFKERTRHLLREWYLQDPYPNP

SEQ ID NO: 88 - protein, 29 aa, Canis lupus
DGEQKTHCFKERTRHLLREWYLQDPYPNP

SEQ ID NO: 89 - protein, 29 aa, Mus musculus
DGEQKTHCFKERTRHLLREWYLQDPYPNP

SEQ ID NO: 90 - protein, 29 aa, Rattus norvegicus
DGEQKTHCFKERTRHLLREWYLQDPYPNP

SEQ ID NO: 91 - protein, 29 aa, Gallus gallus
DGEQKTHCFKERTRHLLREWYLQDPYPNP

SEQ ID NO: 92 - protein, 29 aa, Danio rerio
DGEQKTHCFKERTRHLLREWYLQDPYPNP

SEQ ID NO: 93 - DNA, 23 bp, Artificial Sequence
  Description of Artificial Sequence Synthetic oligonucleotide
accgccgaaagagttcggggcgt

SEQ ID NO: 94 - DNA, 23 bp, Artificial Sequence
  Description of Artificial Sequence Synthetic oligonucleotide
aacacgccccgaactctttcggc

SEQ ID NO: 95 - DNA, 23 bp, Artificial Sequence
  Description of Artificial Sequence Synthetic oligonucleotide
accggtacgaccgaaagagttcg

SEQ ID NO: 96 - DNA, 23 bp, Artificial Sequence
  Description of Artificial Sequence Synthetic oligonucleotide
aaccgaactctttcggtcgtacc

SEQ ID NO: 97 - DNA, 319 bp, Mus musculus
tgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatag

SEQ ID NO: 98 - protein, 50 aa, Artificial Sequence
  Description of Artificial Sequence Synthetic polypeptide
RRRRRRRRRRRRRRRRRRRRRRRRRRRRRRRRRRRRRRRRRRRRRRRRRR

SEQ ID NO: 99 - protein, 9 aa, Artificial Sequence
  Description of Artificial Sequence Synthetic peptide
RRRRRRRRR

SEQ ID NO: 100 - protein, 9 aa, Artificial Sequence
  Description of Artificial Sequence Synthetic peptide
EEEEEEEEE

(19) United States
(12) Patent Application Publication
(10) Pub. No.: US 2017/0072025 A1
(43) Pub. Date: Mar. 16, 2017

(54) METHODS AND COMPOSITIONS FOR THE TREATMENT OF GLAUCOMA

(71) Applicants: YouHealth Biotech, Limited, Grand Cayman (KY); The Regents of the University of California, Oakland, CA (US)

(72) Inventors: Lianghong ZHENG, Shenyang (CN); Jie ZHU, Shenyang (CN); Ling ZHAO, Shenyang (CN); Michael AI, Shenyang (CN); Oulan LI, Shenyang (CN); Kang ZHANG, La Jolla, CA (US)

(21) Appl. No.: 15/261,617

(22) Filed: Sep. 9, 2016

Related U.S. Application Data
(60) Provisional application No. 62/216,374, filed on Sep. 10, 2015.

(51) Int. Cl. A61K 38/46; A61K 9/127; C12N 15/113; A61K 47/48; A61K 9/00

(52) U.S. Cl. CPC A61K 38/465; A61K 47/48092; A61K 9/0048; C12N 15/1135; A61K 9/127; C12N 2310/11; A61K 48/00

(57) ABSTRACT

Disclosed herein are methods and pharmaceutical compositions for the treatment of glaucoma by interfering with expression of genes, such as p16, in cells of the eye. These methods and compositions employ nucleic acid based therapies.

### CROSS-REFERENCE

This application claims the benefit of U.S. Provisional Application No. 62/216,374, filed Sep. 10, 2015, which is incorporated herein by reference.

The instant application contains a Sequence Listing which has been submitted electronically in ASCII format and is hereby incorporated by reference in its entirety. Said ASCII copy, created on Nov. 14, 2016, is named 49697-712_201_SL.txt and is 30,677 bytes in size.

### BACKGROUND OF THE DISCLOSURE

Glaucoma is a blinding neurodegenerative disease. Risk factors for glaucoma include elevated intraocular pressure (IOP), age and genetics. Glaucoma is characterized by accelerated and progressive retinal ganglion cell (RGC) death. Despite decades of research, the mechanism of R(C death in glaucoma is still unknown.

Primary open-angle glaucoma (POAG) is a group of progressive optic neuropathies characterized by a slow and progressive degeneration of retinal ganglion cells (RGCs) and their axons, resulting in a distinct appearance of the optic disc and a concomitant pattern of vision loss (Zhang et al., 2012). POAG is the most frequent type of glaucoma in the western world, one of world's leading causes of blindness, and the leading cause of blindness among African Americans (Kwon et al., 2009). IOP and age are the leading risk factors for both the development and progression of POAG.

### SUMMARY OF THE DISCLOSURE

Disclosed herein are methods of treating a subject for glaucoma or symptoms thereof comprising administering to an eye of the subject: a guide RNA that hybridizes to a target site of a gene, wherein the gene encodes a protein that contributes to glaucoma or symptoms thereof; and a Cas nuclease that cleaves a strand of the gene at the target site, wherein cleaving the strand modifies expression of the gene, thereby reducing contribution of the protein to glaucoma or symptoms thereof. In some embodiments, the method comprises administering a repair template to replace a portion of the gene. In some embodiments, the method comprises reducing expression of the protein or reducing activity of the protein. In some embodiments, the method results in reducing retinal ganglion cell senescence in the eye. In some embodiments, the method comprises administering a polynucleotide encoding the Cas nuclease and the guide RNA in a delivery vehicle selected from a vector, a liposome, and a ribonucleoprotein. In some embodiments, the gene is a p16 gene. In some embodiments, the subject harbors a p16 allelic variant of a wildtype p16 gene, wherein the wildtype p16 gene comprises a coding sequence of SEQ ID NO. 36. In some embodiments, the p16 allelic variant harbors a single nucleotide polymorphism that contributes to glaucoma or symptoms thereof. In some embodiments, the single nucleotide polymorphism is an alanine residue at rs1042522. In some embodiments, the guide RNA targets the Cas nuclease to a sequence of the p16 gene selected from SEQ ID NOS: 17-35. In some embodiments, the gene is a Six6 gene. In some embodiments, the subject harbors a p16 allelic variant of a wildtype p16 gene, wherein the wildtype p16 gene comprises a coding sequence of SEQ ID NO. 37. In some embodiments, the Six6 gene comprises a single nucleotide polymorphism of a cytosine at rs33912345.

Further disclosed herein are methods of treating a subject for glaucoma comprising administering to an eye of the subject an antisense oligonucleotide that hybridizes to a p16 messenger RNA, thereby reducing expression of the p16 gene via RNA interference. In some embodiments, reducing expression of the p16 gene reduces retinal ganglion cell senescence. In some embodiments, retinal ganglion cell senescence is reduced from about 10% to about 90%. In some embodiments, retinal ganglion cell senescence is reduced at least about 40%. In some embodiments, the antisense oligonucleotide is a short hairpin RNA encoded by a sequence selected from SEQ ID NOS: 9-13. In some embodiments, the antisense oligonucleotide is administered in a polynucleotide vector, a liposome, or ribonucleoprotein.

Further disclosed herein are pharmaceutical compositions for the treatment of glaucoma comprising: a polynucleotide encoding a Cas protein; and a guide RNA that is complementary to a portion of a gene selected from a p16 gene and a Six6 gene. In some embodiments, the pharmaceutical composition comprises a repair template, wherein the guide RNA targets the Cas protein to the gene, resulting in Cas-mediated cleavage of the gene and insertion of the repair template. In some embodiments, the polynucleotide encoding the Cas protein and the guide RNA are present in at least one viral vector. In some embodiments, the polynucleotide encoding the Cas protein or the guide RNA are present in a liposome. In some embodiments, the p16 gene comprises a coding sequence of SEQ ID NO: 36. In some embodiments, the portion of the p16 gene comprises a single nucleotide polymorphism of an alanine residue at rs1042522. In some embodiments, the guide RNA targets the Cas nuclease to a sequence of the p16 gene selected from SEQ ID NOS: 17-35. In some embodiments, the Six6 comprises a coding sequence of SEQ ID NO: 37. In some embodiments, the portion of the Six6 gene comprises single nucleotide polymorphism of a cytosine at rs33912345. In some embodiments, the pharmaceutical composition is formulated as a liquid for administration with an eye dropper. In some embodiments, the pharmaceutical composition is formulated as a liquid for intravitreal administration.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a molecular pathway involving Six6 and p16 that can be targeted with therapeutic agents to decrease retinal senescence and blindness that results from glaucoma intraocular pressure.

FIG. 2A-FIG. 2B show SIX6 protein residue 141 variants bind to DNA with similar efficiency. FIG. 2A, Computer modeling of SIX6 structure. Upper panel: model of SIX6 with histidine at position 141; lower panel: model of SIX6 with asparagine at position 141. FIG. 2B shows ChIP-qPCR analysis of SIX6 binding to p27 regulatory element in patient-derived lymphoblastoid cells shows similar efficiency of binding of both SIX6 variants. Experiments repeated 3 times, +/−SD. CTL, negative control; See also FIG. 3A-FIG. 3C.

FIG. 3A-FIG. 3C (related to FIG. 2A-FIG. 2B) show Six6 protein residue 141 variants bind to DNA with similar efficiency. FIG. 3A shows validation of SIX6 antibody specificity by qPCR analysis of ChIP signals on known target. p27 regulatory region shows highly reduced signal in Six6^{-/-} retinas. Experiments repeated 3 times, p-values calculated using a two-tailed Student's t-test (+/−SD; *p<0.05). CTL, negative control. FIG. 3B and FIG. 3C show ChIP-qPCR experiments using either SIX6 antibody (FIG. 3B) or HA antibody (FIG. 3C) after overexpression of HA-tagged SIX6 protein variants show that both forms bind efficiently to the p27 regulatory region. Experiments repeated 3 times, p-values calculated using a two-tailed Student's t-test. +/−SD. CTL, negative control.

FIG. 4A-FIG. 4D show a joint effect of specific alleles of SIX6 (rs33912345) and P16/INK4A (rs3731239) suggest functional interaction between the two genes. FIG. 4A shows results of the logistic regression analysis, plotted as Z-axis by odds ratios. FIG. 4B shows RT-qPCR analysis of mRNA expression of SIX6 and P16/INK4A in human lymphocytes stratified by their SIX6 (rs33912345) genotypes. Four cell lines with rs33912345-AA (non-risk alleles) and four cell lines with rs33912345-CC (risk alleles) were analyzed. Relative mRNA levels were calculated by normalizing results with GAPDH and expressed relative to the AA genotype. p-values were calculated using two-tailed Student's t-test. (+/−SD; *p<0.05, ***p<0.001). FIG. 4C shows SA-βgal staining of human retinas indicating higher numbers of senescent cells in retinas with POAG. FIG. 4D shows quantification of senescent cells in healthy and POAG retinas (*p<0.05); See also FIG. 5.

FIG. 5A-FIG. 5B show increased senescence in human glaucoma retinas. FIG. 5A shows several examples of SA-βgal analysis of the human retina showing higher numbers of senescent cells in retinas with POAG. FIG. 5B shows RT-qPCR analysis of expression of p16/INK4A mRNA in four healthy and four POAG human retinas showing significant upregulation of the p16/INK4A transcript in the diseased retinas. p-values were calculated using a two-tailed Student's t-test (+/−SD; *p<0.05).

FIG. 6A-6F show increased expression of SIX6-risk variant correlates with a higher senescence rate. FIG. 6A shows RT-qPCR analysis shows that overexpression of SIX6-His variant increased p16/INK4A expression in fRPCs. Experiments repeated 3 times, p-values were calculated using two-tailed Student's t-test. (+/−SD, ***p<0.001). CTL, negative control FIG. 6B shows RT-qPCR analysis shows that the overexpression of the SIX6-His variant increased P16/INK4A expression in 293T cells. Experiments repeated 3 times, p-values calculated using a two-tailed Student's t-test. (+/−SD; *p<0.05, **p<0.01). CTL, negative control FIG. 6C shows Western-blot confirming similar levels of expression of both SIX6 variants in transient transfections experiments. CTL, negative control FIG. 6D shows ChIP-qPCR analysis of SIX6 variant association with P16/INK4A promoter, showing similar level of binding. CTL, negative control FIG. 6E shows SA-βgal staining of the fRPCs transfected with either of the two SIX6 variants showing higher ratio of senescence in cells transfected with SIX6-141His risk variant. CTL, negative control FIG. 6F shows quantification of β-galactosidase-positive cells in fRPCs transfected with SIX6 variants. p-values calculated using a two-tailed Student's t-test. (+/−SD; *p<0.05, **p<0.01). CTL, negative control; See also FIG. 7.

FIG. 7 shows induction of IL6, a senescence associated secretory phenotype marker, in immunopanned RGCs upon Six6 protein overexpression. Experiments were repeated 3 times, p-values were calculated using a two-tailed student's t-test. (+/−SD; *p<0.05). CTL, negative control.

FIG. 8A-FIG. 8F show increased expression of SIX6 and induction of cell senescence in retinas upon IOP elevation. FIG. 8A shows the expression of SIX6 protein in mouse retina during development and in the adult stage analyzed by Western blotting. FIG. 8B shows RT-qPCR analysis of Six6 and P16/INK4A mRNA levels shows elevated expression of SIX6 and P16/INK4A in IOP-elevated mouse retinas 5 days after induction of acute experimental glaucoma (5d IOP) as compared to non-treated retina (5d CTL). Experiments were repeated in 8 animals, p-values calculated using a two-tailed Student's t-test. (+/−SD; *p<0.05). FIG. 8C shows ChIP-qPCR analysis of SIX6 protein binding shows its higher association with the P16/INK4A promoter in retinas subjected to acute intraocular pressure increase (5d IOP) as compared to non-treated retina (5d CTL). Experiments repeated 3 times, p-values calculated using a two-tailed Student's t-test. (+/−SD; *p<0.05). CTL, negative control. FIG. 8D shows ChIP-qPCR analysis shows higher levels of p300 association with P16/INK4A promoter upon experimental glaucoma (5d IOP) as compared to non-treated retina (5d CTL). Experiments repeated 3 times, p-values calculated using a two-tailed Student's t-test. (+/−SD; ***p<0.001). CTL, negative control. FIG. 8E shows ChIP-qPCR shows higher level of H3 acetylation at P16/INK4A promoter after acute intraocular pressure increase (5d IOP) as compared to non-treated retina (5d CTL). Experiments repeated 3 times, p-values calculated using a two-tailed Student's t-test (+/−SD; *p<0.05). CTL, negative control. FIG. 8F shows SA-βgal staining of flat mount retinas isolated from treated and non-treated eyes. High number of senescent cells is evident in IOP treated tissue. See also FIG. 8.

FIG. 9A-FIG. 9G show higher expression of Six6 protein and induction of senescence in retinas upon IOP elevation. FIG. 9A and FIG. 9B show RT-qPCR analysis of p19ARF (FIG. 9A) and p15/CDKN2B (FIG. 9B) gene expression in retinas 5 days after TOP-elevation (5d IOP) as compared to non-treated retinas (5d CTL). p-values were calculated using a two-tailed student's t-test (+/−SD; *p<0.05). FIG. 9C shows ChIP-qPCR analysis of WT and Six6^{-/-} retinas showed enrichment of SIX6 protein on p16 regulatory elements. Experiments were repeated 3 times, p-values were calculated using a two-tailed student's t-test (+/−SD; *p<0.05). CTL, negative control. FIG. 9D shows ChIP-qPCR analysis of the recruitment of Six6 protein to the p19ARF and p15/CDKN2B promoters after IOP-elevation (5d IOP) as compared to no-treated retinas (5d CTL). Experiment performed 3 times (+/−SD). CTL, negative control. FIG. 9E shows SA-βgal staining of flat-mounted retinas isolated from treated (5d IOP) and non-treated (5d CTL) eyes show a high number of senescent cells in treated tissue. FIG. 9F and FIG. 9G show quantification of number of SA-βgal positive cells in presented retinas of Mouse #1 and Mouse #2, respectively. p-values were calculated using a two-tailed Student's t-test (+/−SD; **p<0.01, ***p<0.001).

FIG. 10A-FIG. 10E show IOP elevation primarily affects retinal ganglion cells. FIG. 10A, SA-βgal staining of cross-sections in IOP treated (5d IOP) or non-treated (5d CTL) retinas. Senescent cells are localized in the ganglion cell layer (GCL). FIG. 10B, Double staining of TOP treated (5d IOP) or non-treated (5d CTL) flat mount retinas with SA-βgal and BRN3A antibodies. Most senescent cells are also BRN3A positive. FIG. 10C, Immunostaining of TOP-treated Thy1-CFP retinas using anti-GFP antibody. Majority of SA-βgal positive cells are also Thy1-CFP positive. FIG. 10D, Schematic diagram of immunopanning, FIG. 10E, His but not the Asn version of Six6 significantly upregulates p16/INK4A expression as compared to non-transfected (RGC-CTL) or GFP-transfected (RGC-GFP) purified rat retinal ganglion cells. p-values calculated using a two-tailed Student's t-test (+/−SD; *p<0.05). See also FIG. 10.

FIG. 11A-FIG. 11G show IOP-elevation affects retinal ganglion cells. FIG. 11A, Several examples of double, SA-βgal and CFP positive cells in IOP-treated Thy1-CFP retinas. FIG. 11B, Immunostaining of IOP-treated (5d IOP) and untreated (5d CTL) Thy1-CFP retinas using anti-GFP and anti-IL6 antibodies. The number of double-positive cells is specifically increased in IOP-treated tissue. FIG. 11C, Quantification of IL6/CFP double-positive cells in IOP-treated retinas in 3 randomly selected fields (+/−SD). FIG. 11D, RGC purified by immunopanning after 2 days in culture. FIG. 11E, Expression of Brn3a in whole retina cells and immunopanned cells was measured by RT-qPCR. p-values were calculated using a two-tailed Student's t-test, (+/−SD; ***p<0.001). FIG. 11F, Overexpression levels of the His and Asn variants of Six6 in transfected cells was measured using RT-qPCR. +/−SD. FIG. 11G, RT-qPCR analysis of IL6 expression in RGCs upon Six6 variants or GFP (RGC-GFP) overexpression as compared to non-transfected cells (RGC-CTL). p-values were calculated using a two-tailed Student's t-test (+/−SD; *p<0.05).

FIG. 12A-FIG. 12F show absence of either Six6 or P16 protects against RGC death in glaucoma. FIG. 12A and FIG. 12B, show RT-qPCR analysis of Six6 (FIG. 12A) and P16/INK4A (FIG. 12B) mRNA levels upon IOP treatment (5d IOP) shows elevated expression of Six6 and P16/INK4A only in wild-type (Six6^{+/+}) retinas and not in Six6^{+/-} retinas as compared to non-treated (5d CTL) retinas. Experiments repeated in 8 animals, p-values calculated using a two-tailed Student's t-test. (+/−SD; *p<0.05, **p<0.01). FIG. 12C shows SA-β-galactosidase staining of flat mount retinas isolated from IOP-treated and non-treated eyes of Six6^{+/+} and Six6^{+/-} mice shows a lack of senescent cells in the treated tissue isolated from heterozygous mice (blue bar). FIG. 12D, Quantification of the RGC ratio in treated and non-treated retinas in WT and P16^{-/-} mice. FIG. 12E, Quantification of the RGC ratio in IOP treated and non-treated retinas in WT and p53 KO mice. FIG. 12F, Model of the sequence of events leading to RGC death upon Six6 upregulation in glaucoma. See also FIG. 13.

FIG. 13 shows lack of Six6 protects RGCs against senescence. SA-βgal staining of flat-mounted mouse retinas isolated from IOP treated (5d IOP) and non-treated (5d CTL) Six6^{+/-} eyes shows no increase in the number of senescent cells upon IOP elevation, as compared to the Six6^{+/+} eyes (bottom panels) Right panels: quantification of SA-βgal positive cells in presented retinas, +/−SD.

FIG. 14 depicts a schematic representation of an exemplary model for testing p16 gene editing in the retina of a subject for the treatment of glaucoma.

FIG. 15 depicts a schematic representation of a vector encoding a p16 sgRNA and a vector encoding Cas9. ITR: inverted terminal repeats. U6 promoter: a pol III promoter. hSyn: neuron-specific long-term expression promoter. KASH (Klarsicht, ANC-1 and Syne/Nesprin homology): intermediate protein nuclear migration, protein anchorage. hGHpA: human growth hormone polyA. EF1α promoter: Human elongation factor-1 alpha promoter. HA:a tag (Human influenza hemagglutinin). Guide sequences disclosed as SEQ ID NOS 27-28, respectively, in order of appearance.

FIG. 16 shows the number of RGC cells (per high power field) in mice receiving a sham treatment (control, normal eye), elevated intraocular pressure, or elevated ocular pressure after p16 gene editing.

FIG. 17 shows alignment of a p16 exon that is highly conserved between mouse (SEQ ID NO: 97) and human (SEQ ID NO: 1).

### DETAILED DESCRIPTION OF THE DISCLOSURE

Glaucoma is the leading cause of blindness affecting tens of millions of people worldwide. Despite its prevalence, its etiology and pathogenesis are poorly understood and treatment is limited to lowering TOP. Despite aggressive IOP lowering therapies, most patients have progressive loss of visual function and some will eventually become legally blind. There are various types of glaucoma wherein intraocular pressure is elevated, all of which may benefit from the methods and compositions disclosed herein.

SIX6 is a member of the SIX/Sine oculis family of homeobox transcription factors involved in the development of retina. It has been shown to directly regulate expression of cyclin-dependent kinase inhibitor genes during mouse development. Although the role of SIX6 during retina development is being investigated by a number of laboratories, there are very few reports exploring its molecular role in adult retina or in glaucoma pathogenesis. Several SIX6 mutations and single nucleotide polymorphisms (SNPs) have been shown to correlate with developmental eye defects in human. Additionally, several SNPs have been associated with an increased risk of glaucoma. The effects of SIX6 variants had always been assessed after late identification of the disease and this approach does not allow for correct dissociation of developmental defects from the genetic components that cause the disease.

The SIX6 variant (histidine encoding SIX-His), referred to as “risk variant” herein, is actually evolutionary conserved across the phyla. The Asn variant (the protective allele for glaucoma) is detected only in the human branch; however, it is unknown why it would be advantageous for humans to have a protective variant. Several groups performed rescue experiments by overexpressing human His variant in zebrafish and found that it did not rescue eye defects induced by the removal of endogenous SIX6 proteins. Interestingly, both zSix6a and zSix6b carry the His amino acid at the orthologous position. These results suggest that the inability to rescue eye phenotypes by human His version of SIX6 is likely the result of species-specific differences in other residues and not the sole effect of His/Asn variant.

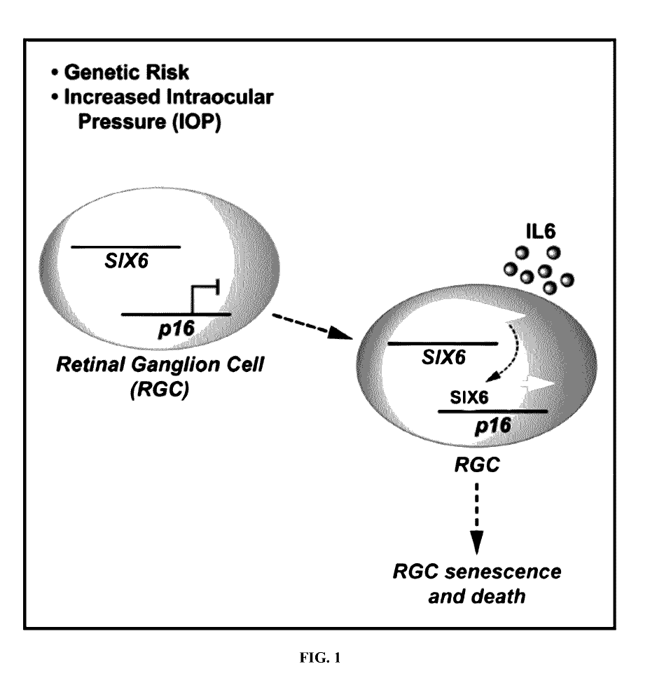

The experiments described herein suggest that cellular senescence plays a critical role in the pathogenesis of glaucoma. Cellular senescence is a state of irreversible growth arrest. When senescent cells accumulate in the tissue, their impaired function can result in a predisposition to disease development and/or progression. As shown herein, SIX6 directly regulates expression of P16/INK4A, an indicator of cell senescence and aging. Further, upon acute IOP elevation, P16/INK4A expression is up-regulated, which, in turn, may be a cause of RGC death (see FIG. 1). Therefore P16/INK4A up-regulation appears to be a downstream integrator of diverse signals such as inherited genetic risk, age and other factors, such as raised IOP. This may provide an explanation for how IOP, the most common risk factor, causes glaucoma. Moreover, it provides a molecular link between genetic susceptibility and other factors to the pathogenesis of glaucoma.

P16/INK4A is classified in the field as a tumor suppressor gene and is encoded by the gene known as cyclin-dependent kinase inhibitor 2A (CDKN2A). For the purpose of the present application, this gene, as well as any RNA or protein transcribed or translated, respectively, therefrom, will be referred to as p16.

As shown herein, SIX6 His variant increases P16/INK4A expression upon increased IOP, which in turn causes RGCs to enter into a senescent state, which may lead to increased RGC death in glaucoma. The experimental results described herein provide important insights into the pathogenesis of glaucoma and prompted the use of the CRISPR/Cas gene editing system to reduce P16/INK4A gene expression.

P16 is a cyclin-dependent kinase inhibitor and a potent negative regulator of cell cycle progression. Consequently, upregulated P16/INK4A expression and senescence phenotype, as measured by SAβ-gal assay and SASP, usually indicate the irreversible cell cycle arrest. Unexpectedly, elevated p16 expression and senescence were observed in retinal ganglion cells (post-mitotic neurons), which are believed not to be replication-competent. Therefore, one possibility is that RGCs may contain a replication-competent, stem cell-like population; alternatively, p16 may play a previously unrecognized role in these post mitotic cells.

In addition to SIX6 and P16/INK4, there may be other factors involved in the development of IOP, RGC death and glaucoma. The experimental results described herein also demonstrate another key player in cellular senescence, P53, contributes to RGC death, as evidenced by protection of RGC from IOP induced damage in mice lacking p53. Additional data show increased expression of secretory molecules, components of Senescence-Associated Secretory Phenotype (SASP), upon IOP-induced retinal damage. Moreover, induction of interleukin 1 (IL1) is an early response to IOP induced RGC damage. Induction of ILL in turn, is known to cause activation of NF-kB dependent senescence associated expression of IL6 and IL8. These data suggest that the senescence-associated cytokine network is activated in TOP-treated retinas. There is also a potential role for other genes located in the 9p21 locus in the pathology of glaucoma. For example, p19ARF may contribute to glaucoma pathogenesis independently by influencing eye vasculature development.

Disclosed herein are nucleic acid therapies for the prevention and treatment of glaucoma. Nucleic acid therapies (e.g., RNAi, CRISPR/Cas) are targeted therapies with high selectivity and specificity. However, in some instances, nucleic acid therapy is also hindered by poor intracellular uptake, limited blood stability and non-specific immune stimulation. To address these issues, various modifications of the nucleic acid composition are explored, such as for example, novel linkers for better stabilizing and/or lower toxicity, optimization of binding moiety for increased target specificity and/or target delivery, and nucleic acid polymer modifications for increased stability and/or reduced off-target effect.

In some embodiments, the arrangement or order of the different components that make-up the nucleic acid composition further effects intracellular uptake, stability, toxicity, efficacy, and/or non-specific immune stimulation. For example, if the nucleic acid component includes a binding moiety, a polymer, and a polynucleic acid molecule (or polynucleotide), the order or arrangement of the binding moiety, the polymer, and/or the polynucleic acid molecule (or polynucleotide) (e.g., binding moiety-polynucleic acid molecule-polymer, binding moiety-polymer-polynucleic acid molecule, or polymer-binding moiety-polynucleic acid molecule) further effects intracellular uptake, stability, toxicity, efficacy, and/or non-specific immune stimulation.

### Therapeutic Platforms

Disclosed herein are methods of treating a subject for glaucoma, comprising administering to the subject a therapeutic agent that inhibits expression of a tumor suppressor gene, wherein the tumor suppressor gene is upregulated in a cell of the eye by intraocular pressure. In some embodiments, the tumor suppressor gene is p16. Further disclosed herein are methods of treating a subject for glaucoma, comprising administering to the subject a therapeutic agent that inhibits a protein that induces senescence of retinal ganglion cells. Disclosed herein are methods of treating a subject for glaucoma, comprising administering to the subject a therapeutic agent that inhibits p16 gene expression or p16 gene expression product (e.g., RNA or protein) expression or activity. Further disclosed herein are methods of treating a subject for glaucoma, comprising administering to the subject a therapeutic agent that inhibits Six6 expression or Six6 gene expression product expression or activity.

In some embodiments, the glaucoma is primary open-angle glaucoma (POAG), also referred to as primary open angle glaucoma, chronic open angle glaucoma, chronic simple glaucoma, and glaucoma simplex. POAG is generally caused by trabecular blockage or clogging of drainage canals in the eye. The examples provided herein demonstrate the usefulness of the methods and compositions described herein for POAG. However, these examples are by no means meant to limit the utility of the invention to POAG. One skilled in the art would easily understand how the methods and compositions disclosed herein would be useful for any condition comprising intraocular pressure, such as a glaucoma.

In some embodiments, the glaucoma is primary angle closure glaucoma, also referred to as primary closed-angle glaucoma, narrow-angle glaucoma, pupil-block glaucoma, acute congestive glaucoma, intermittent angle closure glaucoma, acute angle closure glaucoma, chronic angle closure glaucoma. Primary angle closure glaucoma may be caused by the iris contacting the trabecular meshwork which blocks the flow of aqueous humor from the eye, thereby causing elevated intraocular pressure. In some embodiments, the glaucoma is a developmental glaucoma selected from primary congenital glaucoma, infantile glaucoma, or a hereditary glaucoma (e.g. family history of glaucoma).

In some embodiments, the subject has been diagnosed with glaucoma. In some embodiments, the methods disclosed herein comprise diagnosing the subject with glaucoma. In some embodiments, diagnosing comprises performing at least one method selected from tonometry, anterior chamber angle examination, examination of an optic nerve for visible damage, measurement of cup-to disc ratio, a visual field test, optical coherence tomography, scanning laser polarimetry and scanning laser ophthalmoscopy. In some embodiments, the subject is diagnosed with glaucoma when the subject's eye pressure is greater than or equal to 21 mmHg or 2.8 kPa. Normal eye pressure is generally considered to be between 10 mmHg and 20 mmHg, the average being 15.5 mmHg with fluctuations of about 2.75 mmHg. In some embodiments, the subject is diagnosed with glaucoma when the subject has abnormal optic cupping (e.g., a cup to disc ration of greater than 0.3). In some embodiments, the subject is diagnosed when the subject has risk factors for glaucoma, including, but not limited to, intraocular pressure, a family history of glaucoma, migraines, high blood pressure, obesity, and combinations thereof.

The methods and compositions disclosed herein may reduce a symptom of glaucoma. In some embodiments, the symptom of glaucoma is selected from the group consisting of blindness, decreased vision, blurry vision, a decrease in side vision, decrease in field of vision, a cup-to-disc ratio greater than 0.3, ocular pain, seeing halos around lights, red eye, very high intraocular pressure (>30 mmHg), nausea, vomiting, a fixed mid-dilated pupil, and an oval pupil. In some embodiments, the subject does not experience any ocular pain.

In some embodiments, the methods and compositions disclosed herein may be combined with various known glaucoma therapies for a multi-pronged approach to treating glaucoma. Known therapies for glaucoma include, but are not limited to, medications, laser treatment, and surgery. In some embodiments, the medication comprises a prostaglandin analog, a beta adrenergic receptor antagonist, an alpha2-adrenergic agonist, epinephrine, a mitotic agent, an acetylcholinesterase inhibitor, or a carbonic anhydrase inhibitor. In some embodiments, the laser treatment is selected from Argon laser trabeculoplasty, selective laser trabeculoplasty, Nd:YAG laser peripheral iridotomy, or Diode laser cycloablation. In some embodiments, the surgery is selected from a canaloplasty, trabeculectomy, application of a glaucoma drainage implant, and a sclerectomy. In some embodiments, the methods and compositions disclosed herein are preferable to known glaucoma therapies because they are less invasive, impose less risk to damaging the eye or present fewer side effects than known glaucoma therapies.

### RNAi

In some embodiments, the therapeutic agent is an anti-sense oligonucleotide, a strand of synthesized RNA capable of inhibiting expression of a p16 gene via RNA interference. In some embodiments, the therapeutic agent is an anti-sense oligonucleotide, a strand of synthesized RNA capable of inhibiting expression of a Six6 gene via RNA interference. In some embodiments, the anti-sense oligonucleotide comprises a modification providing resistance to digestion or degradation by naturally-occurring DNase enzymes. In some embodiments, the modification is a modification of the anti-sense oligonucleotide's phosphodiester backbone using a solid-phase phosphoramidite method during its synthesis. This will effectively render most forms of DNase ineffective to the anti-sense oligonucleotide.

In some embodiments, the anti-sense oligonucleotide comprises a delivery system that facilitates or enhances uptake of the anti-sense oligonucleotide most efficiently in two methods. In some embodiments, the delivery system comprises a liposome or lipid container that is easily taken in by a human cell. In some embodiments, the delivery system is a system that is mediated by the tat protein, which allows easy transfer of large molecules, like oligonucleotides, through the cell membrane.

In some embodiments, the anti-sense oligonucleotide is a small hairpin RNA (shRNA). These strands of RNA silence the gene by targeting the mRNA produced by the gene of interest. In some embodiments, the shRNA may be custom-designed via computer software and manufactured commercially using a design template. In some embodiments, the shRNA is delivered using bacterial plasmids, circular strands of bacterial DNA, or viruses carrying viral vectors.

In some embodiments, the anti-sense oligonucleotide targets a RNA encoded by a p16 gene. In some embodiments, the anti-sense oligonucleotide targets a RNA encoded by a Six6 gene. In some embodiments, the anti-sense oligonucleotide targets a RNA encoded by a p53 gene. In some embodiments, the anti-sense oligonucleotide targets a RNA encoded by an IL1 gene. In some embodiments, the anti-sense oligonucleotide targets a RNA encoded by a CDKN2D gene.

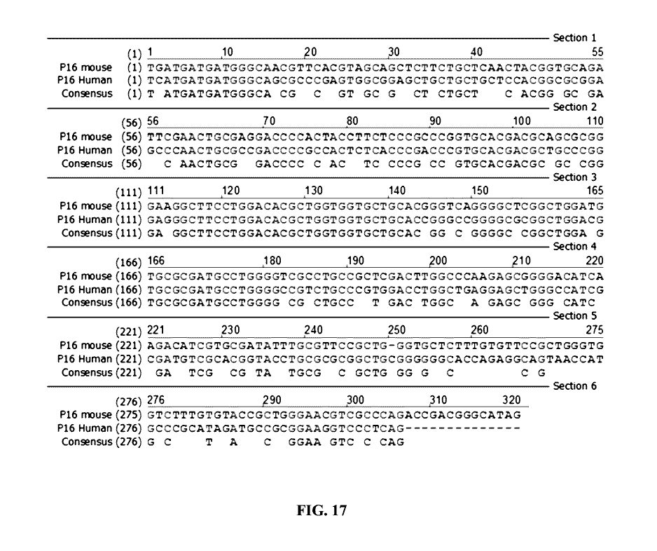

In some embodiments, the anti-sense oligonucleotide is a siRNA or a shRNA that hybridized to a portion of a transcript encoded by the p16 gene, wherein the portion of the transcript is encoded by an exon found at positions 23836-24142 of the human p16 gene (see SEQ ID NO: 1 in Table 7). This exon is highly conserved between mouse p16 and human p16 (see, e.g., FIG. 17).

In some embodiments, the siRNA is between about 18 nucleotides and about 30 nucleotides in length. In some embodiments, the siRNA is 18 nucleotides in length. In some embodiments, the siRNA is 19 nucleotides in length. In some embodiments, the siRNA is 20 nucleotides in length. In some embodiments, the siRNA is 21 nucleotides in length. In some embodiments, the siRNA is 22 nucleotides in length. In some embodiments, the siRNA is 23 nucleotides in length. In some embodiments, the siRNA is 24 nucleotides in length. In some embodiments, the siRNA is 25 nucleotides in length.

In some embodiments, the anti-sense oligonucleotide hybridized to a target sequence of a p16 transcript. In some embodiments, the target sequence is a sequence selected from SEQ ID NOS: 4-8. In some embodiments, the target sequence is encoded by a sequence selected from SEQ ID NOS: 9-13 (see Table 7). In some embodiments, the target sequence is encoded by a sequence that is at least 90% homologous to a sequence selected from SEQ ID NOS: 9-13. In some embodiments, the target sequence is encoded by a sequence that is at least about 80% homologous to a sequence selected from SEQ ID NOS: 9-13. In some embodiments, the target sequence is encoded by a sequence that is at least about 85% homologous to a sequence selected from SEQ ID NOS: 9-13. In some embodiments, the target sequence is encoded by a sequence that is at least about 90% homologous to a sequence selected from SEQ ID NOS: 9-13. In some embodiments, the target sequence is encoded by a sequence that is at least about 95% homologous to a sequence selected from SEQ ID NOS: 9-13.

In some embodiments, the anti-sense oligonucleotide is a shRNA that targets a p16 transcript. In some embodiments, the shRNA is encoded by a sequence selected from SEQ ID NOS: 9-13 (see Table 7). In some embodiments, the shRNA is encoded by a sequence that is at least 90% homologous to a sequence selected from SEQ ID NOS: 9-13. In some embodiments, the shRNA is encoded by a sequence that is at least about 80% homologous to a sequence selected from SEQ ID NOS: 9-13. In some embodiments, the shRNA is encoded by a sequence that is at least about 85% homologous to a sequence selected from SEQ ID NOS: 9-13. In some embodiments, the shRNA is encoded by a sequence that is at least about 90% homologous to a sequence selected from SEQ ID NOS: 9-13. In some embodiments, the shRNA is encoded by a sequence that is at least about 95% homologous to a sequence selected from SEQ ID NOS: 9-13.

### Gene Editing

In some embodiments, methods and cells disclosed herein utilize genome editing to modify a DNA molecule in a cell, for the treatment of glaucoma. In some embodiments, methods and cells disclosed herein utilize genome editing to modify a target gene in a cell, for the treatment of glaucoma. In some embodiments, methods and cells disclosed herein utilize a nuclease or nuclease system. In some embodiments, nuclease systems comprise site-directed nucleases. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute proteins; any derivative thereof; any variant thereof; and any fragment thereof. In some embodiments, site-directed nucleases disclosed herein can be modified in order to generate catalytically dead nucleases that are able to site-specifically bind target sequences without cutting, thereby blocking transcription and reducing target gene expression.

In some embodiments, methods and cells disclosed herein utilize a nucleic acid-guided nuclease system. In some embodiments, methods and cells disclosed herein utilize a clustered regularly interspaced short palindromic repeats (CRISPR), CRISPR-associated (Cas) protein system for modification of a nucleic acid molecule. In some embodiments, the CRISPR/Cas systems disclosed herein comprise a Cas nuclease and a guide RNA. In some embodiments, the CRISPR/Cas systems disclosed herein comprise a Cas nuclease, a guide RNA, and a repair template. The guide RNA directs the Cas nuclease to a target sequence, where the Cas nuclease cleaves or nicks the target sequence, thereby creating a cleavage site. In some embodiments, the Cas nuclease generates a double stranded break (DSB) that is repaired via nonhomology end joining (NHEJ). However, in some embodiments, unmediated or non-directed NHEJ-mediated DSB repair results in disruption of an open reading frame that leads to undesirable consequences. To circumvent these issues, in some embodiments, the methods disclosed herein comprise the use of a repair template to be inserted at the cleavage site, allowing for control of the final edited gene sequence. This use of a repair template may be referred to as homology directed repair (HDR).

In some embodiments, the repair template comprises a wildtype sequence corresponding to the target gene. In some embodiments, the repair template comprises a desired sequence to be delivered to the cleavage site. In some embodiments, the desired sequence is not the wildtype sequence. In some embodiments, the desired sequence is identical to the target sequence with the exception of one or more edited nucleotides to correct or alter the expression/activity of the target gene. For example, the desired sequence may comprise a single nucleotide difference as compared to the target sequence that contained a single nucleotide polymorphism, wherein the single nucleotide difference is a substitution for the nucleotide of the single nucleotide polymorphism that restores wildtype expression/activity or altered expression/activity to the target gene.

Any suitable CRISPR/Cas system may be used for the methods and compositions disclosed herein. The CRISPR/Cas system may be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. The CRISPR/Cas system may be a type I, a type II, a type III, a type IV, a type V, a type VI system, or any other suitable CRISPR/Cas system. The CRISPR/Cas system as used herein may be a Class 1, Class 2, or any other suitably classified CRISPR/Cas system. The Class 1 CRISPR/Cas system may use a complex of multiple Cas proteins to effect regulation. The Class 1 CRISPR/Cas system may comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. The Class 2 CRISPR/Cas system may use a single large Cas protein to effect regulation. The Class 2 CRISPR/Cas systems may comprise, for example, type II (e.g., II, IIA, IIB) and type V CRISPR/Cas type. CRISPR systems may be complementary to each other, and/or can lend functional units in trans to facilitate CRISPR locus targeting.

The Cas protein may be a type I, type II, type III, type IV, type V, or type VI Cas protein. The Cas protein may comprise one or more domains. Non-limiting examples of domains include, a guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. The guide nucleic acid recognition and/or binding domain may interact with a guide nucleic acid. The nuclease domain may comprise catalytic activity for nucleic acid cleavage. The nuclease domain may lack catalytic activity to prevent nucleic acid cleavage. The Cas protein may be a chimeric Cas protein that is fused to other proteins or polypeptides. The Cas protein may be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

The Cas protein may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

The Cas protein may be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia mucimphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida. The term, “derived,” in this instance, is defined as modified from the naturally-occurring variety of bacterial species to maintain a significant portion or significant homology to the naturally-occurring variety of bacterial species. A significant portion may be at least 10 consecutive nucleotides, at least 20 consecutive nucleotides, at least 30 consecutive nucleotides, at least 40 consecutive nucleotides, at least 50 consecutive nucleotides, at least 60 consecutive nucleotides, at least 70 consecutive nucleotides, at least 80 consecutive nucleotides, at least 90 consecutive nucleotides or at least 100 consecutive nucleotides. Significant homology may be at least 50% homologous, at last 60% homologous, at least 70% homologous, at least 80% homologous, at least 90% homologous, or at least 95% homologous. The derived species may be modified while retaining an activity of the naturally-occurring variety.

In some embodiments, the CRISPR/Cas systems utilized by the methods and cells described herein are Type-II CRISPR systems. In some embodiments, the Type-II CRISPR system comprises a repair template to modify the nucleic acid molecule. The Type-II CRISPR system has been described in the bacterium Streptococcus pyogenes, in which Cas9 and two non-coding small RNAs (pre-crRNA and tracrRNA (trans-activating CRISPR RNA)) act in concert to target and degrade a nucleic acid molecule of interest in a sequence-specific manner (see Jinek et al., “A Programmable Dual-RNA-Guided DNA Endonuclease in Adaptive Bacterial Immunity,” Science 337(6096):816-821 (August 2012, epub Jun. 28, 2012)) In some embodiments, the two non-coding small RNAs are connected to create a single nucleic acid molecule, referred to as the guide RNA.

In some embodiments, methods and cells disclosed herein use a guide nucleic acid. The guide nucleic acid refers to a nucleic acid that can hybridize to another nucleic acid. The guide nucleic acid may be RNA. The guide nucleic acid may be DNA. The guide nucleic acid that is DNA may be more stable than a guide RNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The guide nucleic acid may comprise a polynucleotide chain and can be called a “single guide nucleic acid” (i.e. a “single guide nucleic acid”). The guide nucleic acid may comprise two polynucleotide chains and may be called a “double guide nucleic acid” (i.e. a “double guide nucleic acid”). If not otherwise specified, the term “guide nucleic acid” is inclusive, referring to both single guide nucleic acids and double guide nucleic acids.

The guide nucleic acid can comprise a segment that can be referred to as a “guide segment” or a “guide sequence.” The guide nucleic acid may comprise a segment that can be referred to as a “protein binding segment” or “protein binding sequence.”

The guide nucleic acid may comprise one or more modifications (e.g., abuse modification, a backbone modification), to provide the nucleic acid with a new or enhanced feature (e.g., improved stability). The guide nucleic acid may comprise a nucleic acid affinity tag. The guide nucleic acid may comprise a nucleoside. The nucleoside may be a base-sugar combination. The base portion of the nucleoside may be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group may be linked to the 2′, the 3′, or the 5′ hydroxyl moiety of the sugar. In forming guide nucleic acids, the phosphate groups may covalently link adjacent nucleosides to one another to form a linear polymeric compound. In turn, the respective ends of this linear polymeric compound may be further joined to form a circular compound; however, linear compounds are generally suitable. In addition, linear compounds may have internal nucleotide base complementarity and may therefore fold in a manner as to produce a fully or partially double-stranded compound. Within guide nucleic acids, the phosphate groups are commonly referred to as forming the internucleoside backbone of the guide nucleic acid. The linkage or backbone of the guide nucleic acid may be a 3′ to 5′ phosphodiester linkage.

The guide nucleic acid may comprise a modified backbone and/or modified internucleoside linkages. Modified backbones may include those that retain a phosphorus atom in the backbone and those that do not have a phosphorus atom in the backbone.

Suitable modified guide nucleic acid backbones containing a phosphorus atom therein may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

The guide nucleic acid may comprise one or more phosphorothioate and/or heteroatom internucleoside linkages, in particular —CH₂—NH—O—CH₂—, —CH₂—N(CH₃)—O—CH₂— (i.e. a methylene (methylimino) or MMI backbone), —CH₂—O—N(CH₃)—CH₂—, —CH₂—N(CH₃)— N(CH₃)—CH₂— and —O—N(CH₃)—CH₂—CH₂— (wherein the native phosphodiester internucleotide linkage is represented as —O—P(═O)(OH)—O—CH₂—).

The guide nucleic acid may comprise a morpholino backbone structure. For example, the guide nucleic acid may comprise a 6-membered morpholino ring in place of a ribose ring. In some of these embodiments, a phosphorodiamidate or other non-phosphodiester internucleoside linkage replaces a phosphodiester linkage.

The guide nucleic acid may comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These may include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

The guide nucleic acid may comprise a nucleic acid mimetic. The term “mimetic” is intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety may be maintained for hybridization with an appropriate target nucleic acid. One such nucleic acid may be a peptide nucleic acid (PNA). In a PNA, the sugar-backbone of a polynucleotide may be replaced with an amide containing backbone, in particular an aminoethylglycine backbone. The nucleotides may be retained and are bound directly or indirectly to aza nitrogen atoms of the amide portion of the backbone. The backbone in PNA compounds may comprise two or more linked aminoethylglycine units which gives PNA an amide containing backbone. The heterocyclic base moieties may be bound directly or indirectly to aza nitrogen atoms of the amide portion of the backbone.

The guide nucleic acid may comprise linked morpholino units (i.e. morpholino nucleic acid) having heterocyclic bases attached to the morpholino ring. Linking groups c may an link the morpholino monomeric units in a morpholino nucleic acid. Non-ionic morpholino-based oligomeric compounds may have less undesired interactions with cellular proteins. Morpholino-based polynucleotides may be nonionic mimics of guide nucleic acids. A variety of compounds within the morpholino class may be joined using different linking groups. A further class of polynucleotide mimetic may be referred to as cyclohexenyl nucleic acids (CeNA). The furanose ring normally present in a nucleic acid molecule may be replaced with a cyclohexenyl ring. CeNA DMT protected phosphoramidite monomers may be prepared and used for oligomeric compound synthesis using phosphoramidite chemistry. The incorporation of CeNA monomers into a nucleic acid chain may increase the stability of a DNA/RNA hybrid. CeNA oligoadenylates may form complexes with nucleic acid complements with similar stability to the native complexes. A further modification may include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage may be a methylene (—CH2-), group bridging the 2′ oxygen atom and the 4′ carbon atom wherein n is 1 or 2. LNA and LNA analogs may display very high duplex thermal stabilities with complementary nucleic acid (Tm=+3 to +10° C.), stability towards 3′-exonucleolytic degradation and good solubility properties.

The guide nucleic acid may comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH2)nO)mCH₃, O(CH₂)ₙOCH₃, O(CH₂)ₙNH2, O(CH₂)ₙCH₃, O(CH₂)ₙONH₂, and O(CH₂)ₙON((CH₂)ₙCH₃)₂, where n and m are from 1 to about 10. The sugar substituent group may be selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH₂CH₂OCH₃, also known as 2′-O-(2-methoxyethyl) or 2′-MOE i.e., an alkoxyalkoxy group). A further suitable modification may include 2′-dimethylaminooxyethoxy, (i.e., a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE), i.e., 2′-O-CH2-O—CH₂—N(CH₃)₂.

Other suitable sugar substituent groups may include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

The guide nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases may include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties may include those in which the purine or pyrimidine base is replaced with other heterocycles, for example 7-deaza-adenine, 7-deazaguanosine, 2-aminopyridine and 2-pyridone. Nucleobases may be useful for increasing the binding affinity of a polynucleotide compound. These may include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid may comprise chemically linking to the guide nucleic acid one or more moieties or conjugates that can enhance the activity, cellular distribution or cellular uptake of the guide nucleic acid. These moieties or conjugates may include conjugate groups covalently bound to functional groups such as primary or secondary hydroxyl groups. Conjugate groups may include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups may include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties may include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

A modification may include a “Protein Transduction Domain” or PTD (i.e. a cell penetrating peptide (CPP)). The PTD may refer to a polypeptide, polynucleotide, carbohydrate, or organic or inorganic compound that facilitates traversing a lipid bilayer, micelle, cell membrane, organelle membrane, or vesicle membrane. The PTD may be attached to another molecule, which can range from a small polar molecule to a large macromolecule and/or a nanoparticle, and can facilitate the molecule traversing a membrane, for example going from extracellular space to intracellular space, or cytosol to within an organelle. The PTD may be covalently linked to the amino terminus of a polypeptide. The PTD may be covalently linked to the carboxyl terminus of a polypeptide. The PTD may be covalently linked to a nucleic acid. Exemplary PTDs may include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines (SEQ ID NO: 98)), a VP22 domain, a Drosophila Antennapedia protein transduction domain, a truncated human calcitonin peptide, polylysine, and transportan, arginine homopolymer of from 3 arginine residues to 50 arginine residues (SEQ ID NO: 98). The PTD may be an activatable CPP (ACPP). ACPPs can comprise a polycationic CPP (e.g., Arg9 or “R9” (SEQ ID NO: 99)) connected via a cleavable linker to a matching polyanion (e.g., Glu9 or “E9” (SEQ ID NO: 100)), which can reduce the net charge to nearly zero and thereby inhibits adhesion and uptake into cells. Upon cleavage of the linker, the polyanion may be released, locally unmasking the polyarginine and its inherent adhesiveness, thus “activating” the ACPP to traverse the membrane.

The present disclosure provides for guide nucleic acids that can direct the activities of an associated polypeptide (e.g., a site-directed polypeptide) to a specific target sequence within a target nucleic acid. The guide nucleic acid may comprise nucleotides. The guide nucleic acid may be RNA. The guide nucleic acid may be DNA. The guide nucleic acid may comprise a single guide nucleic acid. The guide nucleic acid may comprise a spacer extension and/or a tracrRNA extension. The spacer extension and/or tracrRNA extension may comprise elements that contribute additional functionality (e.g., stability) to the guide nucleic acid. In some embodiments the spacer extension and the tracrRNA extension are optional. The guide nucleic acid may comprise a spacer sequence. The spacer sequence may comprise a sequence that hybridizes to a target nucleic acid sequence. The spacer sequence can be a variable portion of the guide nucleic acid. The sequence of the spacer sequence may be engineered to hybridize to the target nucleic acid sequence. The CRISPR repeat (i.e. referred to in this exemplary embodiment as a minimum CRISPR repeat) may comprise nucleotides that can hybridize to a tracrRNA sequence (i.e. referred to in this exemplary embodiment as a minimum tracrRNA sequence). The minimum CRISPR repeat and the minimum tracrRNA sequence may interact, the interacting molecules comprising a base-paired, double-stranded structure. Together, the minimum CRISPR repeat and the minimum tracrRNA sequence may facilitate binding to the site-directed polypeptide. The minimum CRISPR repeat and the minimum tracrRNA sequence may be linked together to form a hairpin structure through the single guide connector. The 3′ tracrRNA sequence may comprise a protospacer adjacent motif recognition sequence. The 3′ tracrRNA sequence may be identical or similar to part of a tracrRNA sequence. In some embodiments, the 3′ tracrRNA sequence may comprise one or more hairpins.

In some embodiments, the guide nucleic acid may comprise a single guide nucleic acid. The guide nucleic acid may comprise a spacer sequence. The spacer sequence may comprise a sequence that can hybridize to the target nucleic acid sequence. The spacer sequence may be a variable portion of the guide nucleic acid. The spacer sequence may be 5′ of a first duplex. The first duplex may comprise a region of hybridization between a minimum CRISPR repeat and minimum tracrRNA sequence. The first duplex may be interrupted by a bulge. The bulge may comprise unpaired nucleotides. The bulge may be facilitate the recruitment of a site-directed polypeptide to the guide nucleic acid. The bulge may be followed by a first stem. The first stem may comprise a linker sequence linking the minimum CRISPR repeat and the minimum tracrRNA sequence. The last paired nucleotide at the 3′ end of the first duplex may be connected to a second linker sequence. The second linker may comprise a P-domain. The second linker may link the first duplex to a mid-tracrRNA. The mid-tracrRNA may, in some embodiments, comprise one or more hairpin regions. For example the mid-tracrRNA may comprise a second stem and a third stem.

In some embodiments, the guide nucleic acid may comprise a double guide nucleic acid structure. Similar to the single guide nucleic acid structure, the double guide nucleic acid structure may comprise a spacer extension, a spacer, a minimum CRISPR repeat, a minimum tracrRNA sequence, a 3′ tracrRNA sequence, and a tracrRNA extension. However, a double guide nucleic acid may not comprise the single guide connector. Instead the minimum CRISPR repeat sequence may comprise a 3′ CRISPR repeat sequence which may be similar or identical to part of a CRISPR repeat. Similarly, the minimum tracrRNA sequence may comprise a 5′ tracrRNA sequence which may be similar or identical to part of a tracrRNA. The double guide RNAs may hybridize together via the minimum CRISPR repeat and the minimum tracrRNA sequence.

In some embodiments, the first segment (i.e., guide segment) may comprise the spacer extension and the spacer. The guide nucleic acid may guide the bound polypeptide to a specific nucleotide sequence within target nucleic acid via the above mentioned guide segment.

In some embodiments, the second segment (i.e., protein binding segment) may comprise the minimum CRISPR repeat, the minimum tracrRNA sequence, the 3′ tracrRNA sequence, and/or the tracrRNA extension sequence. The protein-binding segment of a guide nucleic acid may interact with a site-directed polypeptide. The protein-binding segment of a guide nucleic acid may comprise two stretches of nucleotides that that may hybridize to one another. The nucleotides of the protein-binding segment may hybridize to form a double-stranded nucleic acid duplex. The double-stranded nucleic acid duplex may be RNA. The double-stranded nucleic acid duplex may be DNA.

In some instances, a guide nucleic acid may comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a guide nucleic acid may comprise, a tracrRNA extension, a 3′tracrRNA sequence, a minimum tracrRNA, a single guide connector, a minimum CRISPR repeat, a spacer, and a spacer extension in any order.

A guide nucleic acid and a site-directed polypeptide may form a complex. The guide nucleic acid may provide target specificity to the complex by comprising a nucleotide sequence that may hybridize to a sequence of a target nucleic acid. In other words, the site-directed polypeptide may be guided to a nucleic acid sequence by virtue of its association with at least the protein-binding segment of the guide nucleic acid. The guide nucleic acid may direct the activity of a Cas9 protein. The guide nucleic acid may direct the activity of an enzymatically inactive Cas9 protein.

Methods of the disclosure may provide for a genetically modified cell. A genetically modified cell may comprise an exogenous guide nucleic acid and/or an exogenous nucleic acid comprising a nucleotide sequence encoding a guide nucleic acid.

Spacer Extension Sequence

A spacer extension sequence may provide stability and/or provide a location for modifications of a guide nucleic acid. A spacer extension sequence may have a length of from about 1 nucleotide to about 400 nucleotides. A spacer extension sequence may have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 40, 1000, 2000, 3000, 4000, 5000, 6000, or 7000 or more nucleotides. A spacer extension sequence may have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. A spacer extension sequence may be less than 10 nucleotides in length. A spacer extension sequence may be between 10 and 30 nucleotides in length. A spacer extension sequence may be between 30-70 nucleotides in length.

The spacer extension sequence may comprise a moiety (e.g., a stability control sequence, an endoribonuclease binding sequence, a ribozyme). The moiety may influence the stability of a nucleic acid targeting RNA. The moiety may be a transcriptional terminator segment (i.e., a transcription termination sequence). The moiety of a guide nucleic acid may have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety may be one that may function in a eukaryotic cell. In some cases, the moiety may be one that may function in a prokaryotic cell. The moiety may be one that may function in both a eukaryotic cell and a prokaryotic cell.

Non-limiting examples of suitable moieties may include: 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A spacer extension sequence may comprise a primer binding site, a molecular index (e.g., barcode sequence). The spacer extension sequence may comprise a nucleic acid affinity tag.

Spacer

The guide segment of a guide nucleic acid may comprise a nucleotide sequence (e.g., a spacer) that may hybridize to a sequence in a target nucleic acid. The spacer of a guide nucleic acid may interact with a target nucleic acid in a sequence-specific manner via hybridization (i.e., base pairing). As such, the nucleotide sequence of the spacer may vary and may determine the location within the target nucleic acid that the guide nucleic acid and the target nucleic acid interact.

The spacer sequence may hybridize to a target nucleic acid that is located 5′ of spacer adjacent motif (PAM). Different organisms may comprise different PAM sequences. For example, in S. pyogenes, the PAM may be a sequence in the target nucleic acid that comprises the sequence 5′-XRR-3′, where R may be either A or G, where X is any nucleotide and X is immediately 3′ of the target nucleic acid sequence targeted by the spacer sequence.

The target nucleic acid sequence may be 20 nucleotides. The target nucleic acid may be less than 20 nucleotides. The target nucleic acid may be at least 5, 10, 15, 16, 17, 18, 19, 20, 21, 22, 23, 24, 25, 30 or more nucleotides. The target nucleic acid may be at most 5, 10, 15, 16, 17, 18, 19, 20, 21, 22, 23, 24, 25, 30 or more nucleotides. The target nucleic acid sequence may be 20 bases immediately 5′ of the first nucleotide of the PAM. For example, in a sequence comprising 5′-NNNNNNNNNNNNNNNNNNNNXRR-3′, the target nucleic acid may be the sequence that corresponds to the N's, wherein N is any nucleotide.

The guide sequence of the spacer that may hybridize to the target nucleic acid may have a length at least about 6 nt. For example, the spacer sequence that may hybridize the target nucleic acid may have a length at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the spacer sequence that may hybridize the target nucleic acid may be 20 nucleotides in length. The spacer that may hybridize the target nucleic acid may be 19 nucleotides in length.

The percent complementarity between the spacer sequence the target nucleic acid may be at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. The percent complementarity between the spacer sequence the target nucleic acid may be at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some cases, the percent complementarity between the spacer sequence and the target nucleic acid may be 100% over the six contiguous 5′-most nucleotides of the target sequence of the complementary strand of the target nucleic acid. In some cases, the percent complementarity between the spacer sequence and the target nucleic acid may be at least 60% over about 20 contiguous nucleotides. In some cases, the percent complementarity between the spacer sequence and the target nucleic acid may be 100% over the fourteen contiguous 5′-most nucleotides of the target sequence of the complementary strand of the target nucleic acid and as low as 0% over the remainder. In such a case, the spacer sequence may be considered to be 14 nucleotides in length. In some cases, the percent complementarity between the spacer sequence and the target nucleic acid may be 100% over the six contiguous 5′-most nucleotides of the target sequence of the complementary strand of the target nucleic acid and as low as 0% over the remainder. In such a case, the spacer sequence may be considered to be 6 nucleotides in length. The target nucleic acid may be more than about 50%, 60%, 70%, 80%, 90%, or 100% complementary to the seed region of the crRNA. The target nucleic acid may be less than about 50%, 60%, 70%, 80%, 90%, or 100% complementary to the seed region of the crRNA.

The spacer segment of a guide nucleic acid may be modified (e.g., by genetic engineering) to hybridize to any desired sequence within a target nucleic acid. For example, a spacer may be engineered (e.g., designed, programmed) to hybridize to a sequence in target nucleic acid that is involved in cancer, cell growth, DNA replication, DNA repair, HLA genes, cell surface proteins, T-cell receptors, immunoglobulin superfamily genes, tumor suppressor genes, microRNA genes, long non-coding RNA genes, transcription factors, globins, viral proteins, mitochondrial genes, and the like.

The spacer sequence may be identified using a computer program (e.g., machine readable code). The computer program may use variables such as predicted melting temperature, secondary structure formation, and predicted annealing temperature, sequence identity, genomic context, chromatin accessibility, % GC, frequency of genomic occurrence, methylation status, presence of SNPs, and the like.

Minimum CRISPR Repeat Sequence

A minimum CRISPR repeat sequence may be a sequence at least about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). The minimum CRISPR repeat sequence may be a sequence with at most about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). The minimum CRISPR repeat may comprise nucleotides that may hybridize to a minimum tracrRNA sequence. The minimum CRISPR repeat and a minimum tracrRNA sequence may form a base-paired, double-stranded structure. Together, the minimum CRISPR repeat and the minimum tracrRNA sequence may facilitate binding to the site-directed polypeptide. A part of the minimum CRISPR repeat sequence may hybridize to the minimum tracrRNA sequence. A part of the minimum CRISPR repeat sequence may be at least about 30%, 40%, 50%, 60%, 65%, 70%, 75%, 80%, 85%, 90%, 95%, or 100% complementary to the minimum tracrRNA sequence. A part of the minimum CRISPR repeat sequence may be at most about 30%, 40%, 50%, 60%, 65%, 70%, 75%, 80%, 85%, 90%, 95%, or 100% complementary to the minimum tracrRNA sequence.

The minimum CRISPR repeat sequence may have a length of from about 6 nucleotides to about 100 nucleotides. For example, the minimum CRISPR repeat sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum CRISPR repeat sequence has a length of approximately 12 nucleotides.

The minimum CRISPR repeat sequence may be at least about 60% identical to a reference minimum CRISPR repeat sequence (e.g., wild type crRNA from S. pyogenes) over a stretch of at least 6, 7, or 8 contiguous nucleotides. The minimum CRISPR repeat sequence may be at least about 60% identical to a reference minimum CRISPR repeat sequence (e.g., wild type crRNA from S. pyogenes) over a stretch of at least 6, 7, or 8 contiguous nucleotides. For example, the minimum CRISPR repeat sequence may be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence may be a sequence with at least about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). The minimum tracrRNA sequence may be a sequence with at most about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). The minimum tracrRNA sequence may comprise nucleotides that may hybridize to a minimum CRISPR repeat sequence. The minimum tracrRNA sequence and a minimum CRISPR repeat sequence may form a base-paired, double-stranded structure. Together, the minimum tracrRNA sequence and the minimum CRISPR repeat may facilitate binding to the site-directed polypeptide. A part of the minimum tracrRNA sequence may hybridize to the minimum CRISPR repeat sequence. A part of the minimum tracrRNA sequence may be 30%, 40%, 50%, 60%, 65%, 70%, 75%, 80%, 85%, 90%, 95%, or 100% complementary to the minimum CRISPR repeat sequence.

The minimum tracrRNA sequence may have a length of from about 6 nucleotides to about 100 nucleotides. For example, the minimum tracrRNA sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum tracrRNA sequence has a length of approximately 14 nucleotides.

The minimum tracrRNA sequence may be at least about 60% identical to a reference minimum tracrRNA (e.g., wild type, tracrRNA from S. pyogenes) sequence over a stretch of at least 6, 7, or 8 contiguous nucleotides. The minimum tracrRNA sequence may be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence may be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum tracrRNA sequence over a stretch of at least 6, 7, or 8 contiguous nucleotides.

The duplex between the minimum CRISPR RNA and the minimum tracrRNA may comprise a double helix. The first base of the first strand of the duplex may be a guanine. The first base of the first strand of the duplex may be an adenine. The duplex between the minimum CRISPR RNA and the minimum tracrRNA may comprise at least about 1, 2, 3, 4, 5, 6, 7, 8, 9, or 10 or more nucleotides. The duplex between the minimum CRISPR RNA and the minimum tracrRNA may comprise at most about 1, 2, 3, 4, 5, 6, 7, 8, 9, or 10 or more nucleotides.

The duplex may comprise a mismatch. The duplex may comprise at least about 1, 2, 3, 4, or 5 or mismatches. The duplex may comprise at most about 1, 2, 3, 4, or 5 or mismatches. In some instances, the duplex comprises no more than 2 mismatches.

Bulge

A bulge may refer to an unpaired region of nucleotides within the duplex made up of the minimum CRISPR repeat and the minimum tracrRNA sequence. The bulge may be important in the binding to the site-directed polypeptide. A bulge may comprise, on one side of the duplex, an unpaired 5′-XXXY-3′ where X is any purine and Y may be a nucleotide that may form a wobble pair with a nucleotide on the opposite strand, and an unpaired nucleotide region on the other side of the duplex.

For example, the bulge may comprise an unpaired purine (e.g., adenine) on the minimum CRISPR repeat strand of the bulge. In some embodiments, a bulge may comprise an unpaired 5′-AAGY-3′ of the minimum tracrRNA sequence strand of the bulge, where Y may be a nucleotide that may form a wobble pairing with a nucleotide on the minimum CRISPR repeat strand.

A bulge on a first side of the duplex (e.g., the minimum CRISPR repeat side) may comprise at least 1, 2, 3, 4, or 5 or more unpaired nucleotides. A bulge on a first side of the duplex (e.g., the minimum CRISPR repeat side) may comprise at most 1, 2, 3, 4, or 5 or more unpaired nucleotides. A bulge on the first side of the duplex (e.g., the minimum CRISPR repeat side) may comprise 1 unpaired nucleotide.

A bulge on a second side of the duplex (e.g., the minimum tracrRNA sequence side of the duplex) may comprise at least 1, 2, 3, 4, 5, 6, 7, 8, 9, or 10 or more unpaired nucleotides. A bulge on a second side of the duplex (e.g., the minimum tracrRNA sequence side of the duplex) may comprise at most 1, 2, 3, 4, 5, 6, 7, 8, 9, or 10 or more unpaired nucleotides. A bulge on a second side of the duplex (e.g., the minimum tracrRNA sequence side of the duplex) may comprise 4 unpaired nucleotides.

Regions of different numbers of unpaired nucleotides on each strand of the duplex may be paired together. For example, a bulge may comprise 5 unpaired nucleotides from a first strand and 1 unpaired nucleotide from a second strand. A bulge may comprise 4 unpaired nucleotides from a first strand and 1 unpaired nucleotide from a second strand. A bulge may comprise 3 unpaired nucleotides from a first strand and 1 unpaired nucleotide from a second strand. A bulge may comprise 2 unpaired nucleotides from a first strand and 1 unpaired nucleotide from a second strand. A bulge may comprise 1 unpaired nucleotide from a first strand and 1 unpaired nucleotide from a second strand. A bulge may comprise 1 unpaired nucleotide from a first strand and 2 unpaired nucleotides from a second strand. A bulge may comprise 1 unpaired nucleotide from a first strand and 3 unpaired nucleotides from a second strand. A bulge may comprise 1 unpaired nucleotide from a first strand and 4 unpaired nucleotides from a second strand. A bulge may comprise 1 unpaired nucleotide from a first strand and 5 unpaired nucleotides from a second strand.

In some instances a bulge may comprise at least one wobble pairing. In some instances, a bulge may comprise at most one wobble pairing. A bulge sequence may comprise at least one purine nucleotide. A bulge sequence may comprise at least 3 purine nucleotides. A bulge sequence may comprise at least 5 purine nucleotides. A bulge sequence may comprise at least one guanine nucleotide. A bulge sequence may comprise at least one adenine nucleotide.

P-Domain (P-DOMAIN)

A P-domain may refer to a region of a guide nucleic acid that may recognize a protospacer adjacent motif (PAM) in a target nucleic acid. A P-domain may hybridize to a PAM in a target nucleic acid. As such, a P-domain may comprise a sequence that is complementary to a PAM. A P-domain may be located 3′ to the minimum tracrRNA sequence. A P-domain may be located within a 3′ tracrRNA sequence (i.e., a mid-tracrRNA sequence).

A p start at least about 1, 2, 3, 4, 5, 6, 7, 8, 9, 10, 15, or 20 or more nucleotides 3′ of the last paired nucleotide in the minimum CRISPR repeat and minimum tracrRNA sequence duplex. A P-domain may start at most about 1, 2, 3, 4, 5, 6, 7, 8, 9 or 10 or more nucleotides 3′ of the last paired nucleotide in the minimum CRISPR repeat and minimum tracrRNA sequence duplex.

A P-domain may comprise at least about 1, 2, 3, 4, 5, 6, 7, 8, 9, 10, 15, or 20 or more consecutive nucleotides. A P-domain may comprise at most about 1, 2, 3, 4, 5, 6, 7, 8, 9, 10, 15, or 20 or more consecutive nucleotides.

In some instances, a P-domain may comprise a CC dinucleotide (i.e., two consecutive cytosine nucleotides). The CC dinucleotide may interact with the GG dinucleotide of a PAM, wherein the PAM comprises a 5′-XGG-3′ sequence.

A P-domain may be a nucleotide sequence located in the 3′ tracrRNA sequence (i.e., mid-tracrRNA sequence). A P-domain may comprise duplexed nucleotides (e.g., nucleotides in a hairpin, hybridized together. For example, a P-domain may comprise a CC dinucleotide that is hybridized to a GG dinucleotide in a hairpin duplex of the 3′ tracrRNA sequence (i.e., mid-tracrRNA sequence). The activity of the P-domain (e.g., the guide nucleic acid's ability to target a target nucleic acid) may be regulated by the hybridization state of the P-DOMAIN. For example, if the P-domain is hybridized, the guide nucleic acid may not recognize its target. If the P-domain is unhybridized the guide nucleic acid may recognize its target.

The P-domain may interact with P-domain interacting regions within the site-directed polypeptide. The P-domain may interact with an arginine-rich basic patch in the site-directed polypeptide. The P-domain interacting regions may interact with a PAM sequence. The P-domain may comprise a stem loop. The P-domain may comprise a bulge.

3′tracrRNA Sequence

A 3′tracr RNA sequence may be a sequence with at least about 30%, 40%, 50%, 60%, 65%, 70%, 75%, 80%, 85%, 90%, 95%, or 100% sequence identity and/or sequence homology with a reference tracrRNA sequence (e.g., a tracrRNA from S. pyogenes). A 3′tracr RNA sequence may be a sequence with at most about 30%, 40%, 50%, 60%, 65%, 70%, 75%, 80%, 85%, 90%, 95%, or 100% sequence identity and/or sequence homology with a reference tracrRNA sequence (e.g., tracrRNA from S. pyogenes).

The 3′ tracrRNA sequence may have a length of from about 6 nucleotides to about 100 nucleotides. For example, the 3′ tracrRNA sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the 3′ tracrRNA sequence has a length of approximately 14 nucleotides.

The 3′ tracrRNA sequence may be at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence may be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

A 3′ tracrRNA sequence may comprise more than one duplexed region (e.g., hairpin, hybridized region). A 3′ tracrRNA sequence may comprise two duplexed regions.

The 3′ tracrRNA sequence may also be referred to as the mid-tracrRNA. The mid-tracrRNA sequence may comprise a stem loop structure. In other words, the mid-tracrRNA sequence may comprise a hairpin that is different than a second or third stems. A stem loop structure in the mid-tracrRNA (i.e., 3′ tracrRNA) may comprise at least 1, 2, 3, 4, 5, 6, 7, 8, 9, 10, 15 or 20 or more nucleotides. A stem loop structure in the mid-tracrRNA (i.e., 3′ tracrRNA) may comprise at most 1, 2, 3, 4, 5, 6, 7, 8, 9 or 10 or more nucleotides. The stem loop structure may comprise a functional moiety. For example, the stem loop structure may comprise an aptamer, a ribozyme, a protein-interacting hairpin, a CRISPR array, an intron, and an exon. The stem loop structure may comprise at least about 1, 2, 3, 4, or 5 or more functional moieties. The stem loop structure may comprise at most about 1, 2, 3, 4, or 5 or more functional moieties.

The hairpin in the mid-tracrRNA sequence may comprise a P-domain. The P-domain may comprise a double stranded region in the hairpin.

tracrRNA Extension Sequence

A tracrRNA extension sequence may provide stability and/or provide a location for modifications of a guide nucleic acid. The tracrRNA extension sequence may have a length of from about 1 nucleotide to about 400 nucleotides. The tracrRNA extension sequence may have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence may have a length from about 20 to about 5000 or more nucleotides. The tracrRNA extension sequence may have a length of more than 1000 nucleotides. The tracrRNA extension sequence may have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 nucleotides. The tracrRNA extension sequence may have a length of less than 1000 nucleotides. The tracrRNA extension sequence may be less than 10 nucleotides in length. The tracrRNA extension sequence may be between 10 and 30 nucleotides in length. The tracrRNA extension sequence may be between 30-70 nucleotides in length.

The tracrRNA extension sequence may comprise a moiety (e.g., stability control sequence, ribozyme, endoribonuclease binding sequence). A moiety may influence the stability of a nucleic acid targeting RNA. A moiety may be a transcriptional terminator segment (i.e., a transcription termination sequence). A moiety of a guide nucleic acid may have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety may be one that may function in a eukaryotic cell. In some cases, the moiety may be one that may function in a prokaryotic cell. The moiety may be one that may function in both a eukaryotic cell and a prokaryotic cell.

Non-limiting examples of suitable tracrRNA extension moieties include: a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A tracrRNA extension sequence may comprise a primer binding site, a molecular index (e.g., barcode sequence). In some embodiments of the disclosure, the tracrRNA extension sequence may comprise one or more affinity tags.

Single Guide Nucleic Acid

The guide nucleic acid may be a single guide nucleic acid. The single guide nucleic acid may be RNA. A single guide nucleic acid may comprise a linker between the minimum CRISPR repeat sequence and the minimum tracrRNA sequence that may be called a single guide connector sequence.

The single guide connector of a single guide nucleic acid may have a length of from about 3 nucleotides to about 100 nucleotides. For example, the linker may have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt. For example, the linker may have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single guide nucleic acid is between 4 and 40 nucleotides. The linker may have a length at least about 100, 500, 1000, 1500, 2000, 2500, 3000, 3500, 4000, 4500, 5000, 5500, 6000, 6500, or 7000 or more nucleotides. The linker may have a length at most about 100, 500, 1000, 1500, 2000, 2500, 3000, 3500, 4000, 4500, 5000, 5500, 6000, 6500, or 7000 or more nucleotides.

The linker sequence may comprise a functional moiety. For example, the linker sequence may comprise an aptamer, a ribozyme, a protein-interacting hairpin, a CRISPR array, an intron, and an exon. The linker sequence may comprise at least about 1, 2, 3, 4, or 5 or more functional moieties. The linker sequence may comprise at most about 1, 2, 3, 4, or 5 or more functional moieties.

In some embodiments, the single guide connector may connect the 3′ end of the minimum CRISPR repeat to the 5′ end of the minimum tracrRNA sequence. Alternatively, the single guide connector may connect the 3′ end of the tracrRNA sequence to the 5′ end of the minimum CRISPR repeat. That is to say, a single guide nucleic acid may comprise a 5′ DNA-binding segment linked to a 3′ protein-binding segment. A single guide nucleic acid may comprise a 5′ protein-binding segment linked to a 3′ DNA-binding segment.

The guide nucleic acid may comprise a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6, 7, or 8 contiguous nucleotides and wherein the minimum CRISPR repeat has a length from 5-30 nucleotides; a minimum tracrRNA sequence comprising at least 60% identity to a tracrRNA from a bacterium (e.g., S. pyogenes) over 6, 7, or 8 contiguous nucleotides and wherein the minimum tracrRNA sequence has a length from 5-30 nucleotides; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6, 7, or 8 contiguous nucleotides and wherein the 3′ tracrRNA comprises a length from 10-20 nucleotides, and comprises a duplexed region; and/or a tracrRNA extension comprising 10-5000 nucleotides in length, or any combination thereof. This guide nucleic acid may be referred to as a single guide nucleic acid.

The guide nucleic acid may comprise a spacer extension sequence from 10-5000 nucleotides in length; a spacer sequence of 12-30 nucleotides in length, wherein the spacer is at least 50% complementary to a target nucleic acid; a duplex comprising 1) a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, 2) a minimum tracrRNA sequence comprising at least 60% identity to a tracrRNA from a bacterium (e.g., S. pyogenes) over 6 contiguous nucleotides and wherein the minimum tracrRNA sequence has a length from 5-30 nucleotides, and 3) a bulge wherein the bulge comprises at least 3 unpaired nucleotides on the minimum CRISPR repeat strand of the duplex and at least 1 unpaired nucleotide on the minimum tracrRNA sequence strand of the duplex; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6 contiguous nucleotides, wherein the 3′ tracrRNA comprises a length from 10-20 nucleotides and comprises a duplexed region; a P-domain that starts from 1-5 nucleotides downstream of the duplex comprising the minimum CRISPR repeat and the minimum tracrRNA, comprises 1-10 nucleotides, comprises a sequence that may hybridize to a protospacer adjacent motif in a target nucleic acid, may form a hairpin, and is located in the 3′ tracrRNA region; and/or a tracrRNA extension comprising 10-5000 nucleotides in length, or any combination thereof.

Double Guide Nucleic Acid

The guide nucleic acid may be a double guide nucleic acid. The double guide nucleic acid can be RNA. The double guide nucleic acid can comprise two separate nucleic acid molecules (i.e. polynucleotides). Each of the two nucleic acid molecules of a double guide nucleic acid can comprise a stretch of nucleotides that can hybridize to one another such that the complementary nucleotides of the two nucleic acid molecules hybridize to form the double stranded duplex of the protein-binding segment. If not otherwise specified, the term “guide nucleic acid” can be inclusive, referring to both single-molecule guide nucleic acids and double-molecule guide nucleic acids.

The double guide nucleic acid may comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; and a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides; and 2) a second nucleic acid molecule of the double-guide nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides; a 3′ tracrRNA that comprises at least 60% identity to a tracrRNA from a bacterium (e.g., S. pyogenes) over 6 contiguous nucleotides and wherein the 3′ tracrRNA comprises a length from 10-20 nucleotides, and comprises a duplexed region; and/or a tracrRNA extension comprising 10-5000 nucleotides in length, or any combination thereof.

In some instances, the double-guide nucleic acid may comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, and at least 3 unpaired nucleotides of a bulge; and 2) a second nucleic acid molecule of the double-guide nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides and at least 1 unpaired nucleotide of a bulge, wherein the 1 unpaired nucleotide of the bulge is located in the same bulge as the 3 unpaired nucleotides of the minimum CRISPR repeat; a 3′ tracrRNA that comprises at least 60% identity to a tracrRNA from a prokaryote (e.g., S. pyogenes) or phage over 6 contiguous nucleotides and wherein the 3′ tracrRNA comprises a length from 10-20 nucleotides, and comprises a duplexed region; a P-domain that starts from 1-5 nucleotides downstream of the duplex comprising the minimum CRISPR repeat and the minimum tracrRNA, comprises 1-10 nucleotides, comprises a sequence that can hybridize to a protospacer adjacent motif in a target nucleic acid, can form a hairpin, and is located in the 3′ tracrRNA region; and/or a tracrRNA extension comprising 10-5000 nucleotides in length, or any combination thereof.

Complex of a Guide Nucleic Acid and a Site-Directed Polypeptide

The guide nucleic acid may interact with a site-directed polypeptide (e.g., a nucleic acid-guided nucleases, Cas9), thereby forming a complex. The guide nucleic acid may guide the site-directed polypeptide to a target nucleic acid.

In some embodiments, the guide nucleic acid may be engineered such that the complex (e.g., comprising a site-directed polypeptide and a guide nucleic acid) can bind outside of the cleavage site of the site-directed polypeptide. In this case, the target nucleic acid may not interact with the complex and the target nucleic acid can be excised (e.g., free from the complex).

In some embodiments, the guide nucleic acid may be engineered such that the complex can bind inside of the cleavage site of the site-directed polypeptide. In this case, the target nucleic acid can interact with the complex and the target nucleic acid can be bound (e.g., bound to the complex).

Any guide nucleic acid of the disclosure, a site-directed polypeptide of the disclosure, an effector protein, a multiplexed genetic targeting agent, a donor polynucleotide, a tandem fusion protein, a reporter element, a genetic element of interest, a component of a split system and/or any nucleic acid or proteinaceous molecule necessary to carry out the embodiments of the methods of the disclosure may be recombinant, purified and/or isolated.

In some embodiments, the methods comprise using a CRISPR/Cas system to modify a mutation in the nucleic acid molecule. In some embodiments, the mutation is a substitution, insertion, or deletion. In some embodiments, the mutation is a single nucleotide polymorphism.

In some cases, the target sequence is between 10 to 30 nucleotides in length. In some instances, the target sequence is between 15 to 30 nucleotides in length. In some cases, the target sequence is about 11, 12, 13, 14, 15, 16, 17, 18, 19, 20, 21, 22, 23, 24, 25, 26, 27, 28, 29, or 30 nucleotides in length. In some cases, the target sequence is about 15, 16, 17, 18, 19, 20, 21, or 22 nucleotides in length.

In some instances, a CRISPR/Cas system utilizes a Cas9 enzyme or a variant thereof. In some embodiments, the methods and cell disclosed herein utilize a polynucleotide encoding the Cas9 enzyme or the variant thereof. In some embodiments, the Cas9 is a double stranded nuclease with two active cutting sites, one for each strand of the double helix. In some instances, the Cas9 enzyme or variant thereof generates a double-stranded break. In some embodiments, the Cas9 enzyme is a wildtype Cas9 enzyme. In some embodiments, the Cas9 enzyme is a naturally-occurring variant or mutant of the wildtype Cas9 enzyme or S. pyogenes Cas9 enzyme. The variant may be an enzyme that is partially homologous to a wildtype Cas9 enzyme, while maintaining Cas9 nuclease activity. The variant may be an enzyme that only comprises a portion of the wildtype Cas9 enzyme, while maintaining Cas9 nuclease activity. In some embodiments, the wildtype Cas9 enzyme is a Streptococcus pyogenes (S. pyogenes) Cas9 enzyme. In some embodiments, the wildtype Cas9 enzyme is represented by an amino acid sequence given GenBank ID AKP81606.1. In some embodiments, the variant is at least about 95% homologous to the amino acid sequence given GenBank ID AKP81606.1. In some embodiments, the variant is at least about 90% homologous to the amino acid sequence given GenBank ID AKP81606.1. In some embodiments, the variant is at least about 80% homologous to the amino acid sequence given GenBank ID AKP81606.1. In some embodiments, the variant is at least about 70% homologous to the amino acid sequence given GenBank ID AKP81606.1. In some instances, the Cas9 enzyme is an optimized Cas9 enzyme, modified from the wild-type Cas9 enzyme for optimal expression and/or activity in the cells described herein. In some embodiments, the Cas9 enzyme is a modified Cas9 enzyme, wherein the modified Cas9 enzyme comprises a Cas9 enzyme or variant thereof as described herein and an additional amino acid sequence. The additional amino acid sequence, by way of non-limiting example, may provide an additional activity, stability, or identifying tag/barcode to the Cas9 enzyme or variant thereof.

The naturally-occurring S. pyogenes Cas9 enzyme cleaves DNA to generate a double stranded break. In some embodiments, the Cas9 enzymes disclosed herein function as a Cas9 nickase, wherein the Cas9 nickase is a Cas9 enzyme that has been modified to nick the target sequence, creating a single stranded break. In some embodiments, the methods disclosed herein comprise use of the Cas9 nickase with more than one guide RNA targeting the target sequence to cleave each DNA strand in a staggered pattern at the target sequence. In some embodiments, using two guide RNAs with Cas9 nickase may increase the target specificity of the CRISPR/Cas systems disclosed herein. In some embodiments, using two or more guide RNAs may result in generating a genomic deletion. In some embodiments, the genomic deletion is a deletion of about 5 nucleotides to about 50,000 nucleotides. In some embodiments, the genomic deletion is a deletion of about 5 nucleotides to about 1,000 nucleotides. In some embodiments, the methods disclosed herein comprise using a plurality of guide RNAs. In some embodiments, the plurality of guide RNAs targets a single gene. In some embodiments, the plurality of guide RNAs targets a plurality of genes.

In some instances, the specificity of the guide RNA for the target sequence is about 80%, 85%, 90%, 95%, 96%, 97%, 98%, 99%, or higher. In some instances, the guide RNA has less than about 20%, 15%, 10%, 5%, 3%, 1%, or less off-target binding rate.

In some embodiments, the specificity of the guide RNA that hybridizes to the target sequence has about 95%, 98%, 99%, 99.5% or 100% sequence complementarity to the target sequence. In some instances, the hybridization is a high stringent hybridization condition.

In some embodiments, the guide RNA targets the nuclease to a p16 gene. In some embodiments, the guide RNA comprises a sequence that hybridizes to a target sequence of the p16 gene. In some embodiments, the target sequence selected from SEQ ID NOS: 17-35. In some embodiments, the target sequence is at least 90% homologous to a sequence selected from SEQ ID NOS: 17-35. In some embodiments, the target sequence is at least about 80% homologous to a sequence selected from SEQ ID NOS: 17-35. In some embodiments, the target sequence is at least about 85% homologous to a sequence selected from SEQ ID NOS: 17-35. In some embodiments, the target sequence is at least about 90% homologous to a sequence selected from SEQ ID NOS: 17-35. In some embodiments, the target sequence is at least about 95% homologous to a sequence selected from SEQ ID NOS: 17-35.

DNA-Guided Nucleases

In some embodiments, methods and cells disclosed herein utilize a nucleic acid-guided nuclease system. In some embodiments, the methods and cells disclosed herein use DNA-guided nuclease systems. In some embodiments, the methods and cells disclosed herein use Argonaute systems.

An Argonaute protein may be a polypeptide that can bind to a target nucleic acid. The Argonaute protein may be a nuclease. The Argonaute protein may be a eukaryotic, prokaryotic, or archaeal Argonaute protein. The Argonaute protein may be a prokaryotic Argonaute protein (pArgonaute). The pArgonaute may be derived from an archaea. The pArgonaute may be derived from a bacterium. The bacterium may be selected from a thermophilic bacterium and a mesophilic bacterium. The bacteria or archaea may be selected from Aquifex aeolicus, Microsystis aeruginosa, Clostridium bartlettii, Exiguobacterium, Anoxybacillus flavithermus, Halogeometricum borinquense, Halorubrum lacusprofundi, Aromatoleum aromaticum, Thermus thermophilus, Synechococcus, Synechococcus elongatus, and Thermosynechococcus elogatus, or any combination thereof. The bacterium may be a thermophilic bacterium. The bacterium may be Aquifex aeolicus. The thermophilic bacterium may be Thermus thermophilus (T. thermophilus) (TtArgonaute). The Argonaute may be from a Synechococcus bacterium. The Argonaute may be from Synechococcus elongatus. The pArgonaute may be a variant pArgonaute of a wild-type pArgonaute.

In some embodiments, the Argonaute of the disclosure is a type I prokaryotic Argonaute (pAgo). In some embodiments, the type I prokaryotic Argonaute carries a DNA nucleic acid-targeting nucleic acid. In some embodiments, the DNA nucleic acid-targeting nucleic acid targets one strand of a double stranded DNA (dsDNA) to produce a nick or a break of the dsDNA. In some embodiments, the nick or break triggers host DNA repair. In some embodiments, the host DNA repair is non-homologous end joining (NHEJ) or homologous directed recombination (HDR). In some embodiments, the dsDNA is selected from a genome, a chromosome and a plasmid. In some embodiments, the type I prokaryotic Argonaute is a long type I prokaryotic Argonaute. In some embodiments, the long type I prokaryotic Argonaute possesses an N-PAZ-MID-PIWI domain architecture. In some embodiments the long type I prokaryotic Argonaute possesses a catalytically active PIWI domain. In some embodiments, the long type I prokaryotic Argonaute possesses a catalytic tetrad encoded by aspartate-glutamate-aspartate-aspartate/histidine (DEDX). In some embodiments, the catalytic tetrad binds one or more Mg+ ions. In some embodiments, the catalytic tetrad does not bind Mg+ ions. In some embodiments, the catalytic tetrad binds one or more Mn+ ions. In some embodiments, the catalytically active PIWI domain is optimally active at a moderate temperature. In some embodiments, the moderate temperature is about 25° C. to about 45° C. In some embodiments, the moderate temperature is about 37° C. In some embodiments, the type I prokaryotic Argonaute anchors the 5′ phosphate end of a DNA guide. In some embodiments, the DNA guide has a deoxy-cytosine at its 5′ end. In some embodiments, the type I prokaryotic Argonaute is a Thermus thermophilus Ago (TtAgo). In some embodiments, the type I prokaryotic Argonaute is a Synechococcus elongatus Ago (SeAgo).

In some embodiments, the prokaryotic Argonaute is a type II pAgo. In some embodiments, the type II prokaryotic Argonaute carries an RNA nucleic acid-targeting nucleic acid. In some embodiments, the RNA nucleic acid-targeting nucleic acid targets one strand of a double stranded DNA (dsDNA) to produce a nick or a break of the dsDNA. In some embodiments, the nick or break triggers host DNA repair. In some embodiments, the host DNA repair is non-homologous end joining (NHEJ) or homologous directed recombination (HDR). In some embodiments, the dsDNA is selected from a genome, a chromosome and a plasmid. In some embodiments, the type II prokaryotic Argonaute is selected from a long type II prokaryotic Argonaute and a short type II prokaryotic Argonaute. In some embodiments, the long type II prokaryotic Argonaute has an N-PAZ-MID-PIWI domain architecture. In some embodiments, the long type II prokaryotic Argonaute does not have an N-PAZ-MID-PIWI domain architecture. In some embodiments, the short type II prokaryotic Argonaute has a MID and PIWI domain, but not a PAZ domain. In some embodiments, the short type II pAgo has an analog of a PAZ domain. In some embodiments the type II pAgo does not have a catalytically active PIWI domain. In some embodiments, the type II pAgo lacks a catalytic tetrad encoded by aspartate-glutamate-aspartate-aspartate/histidine (DEDX). In some embodiments, a gene encoding the type II prokaryotic Argonaute clusters with one or more genes encoding a nuclease, a helicase or a combination thereof. The nuclease or helicase may be natural, designed or a domain thereof. In some embodiments, the nuclease is selected from a Sir2, RE1 and TIR. In some embodiments, the type II pAgo anchors the 5′ phosphate end of an RNA guide. In some embodiments, the RNA guide has a uracil at its 5′ end. In some embodiments, the type II prokaryotic Argonaute is a Rhodobacter sphaeroides Argonaute (RsAgo).

In some embodiments, a pair of pAgos can carry RNA and/or DNA nucleic acid-targeting nucleic acid. A type I pAgo can carry an RNA nucleic acid-targeting nucleic acid, each capable of targeting one strand of a double stranded DNA to produce a double-stranded break in the double stranded DNA. In some embodiments, the pair of pAgos comprises two type I pAgos. In some embodiments, the pair of pAgos comprises two type II pAgos. In some embodiments, the pair of pAgos comprises a type I pAgo and a type II pAgo.

Argonaute proteins can be targeted to target nucleic acid sequences by a guiding nucleic acid.

The guiding nucleic acid can be single stranded or double stranded. The guiding nucleic acid can be DNA, RNA, or a DNA/RNA hybrid. The guiding nucleic acid can comprise chemically modified nucleotides.

The guiding nucleic acid can hybridize with the sense or antisense strand of a target polynucleotide.

The guiding nucleic acid can have a 5′ modification. 5′ modifications can be phosphorylation, methylation, hydroxymethylation, acetylation, ubiquitylation, or sumolyation. The 5′ modification can be phosphorylation.

The guiding nucleic acid can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs in length. In some examples, the guiding nucleic acid can be less than 10 nucleotides or base pairs in length. In some examples, the guiding nucleic acid can be more than 50 nucleotides or base pairs in length.

The guiding nucleic acid can be a guide DNA (gDNA). The gDNA can have a 5′ phosphorylated end. The gDNA can be single stranded or double stranded. The gDNA can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs in length. In some examples, the gDNA can be less than 10 nucleotides in length. In some examples, the gDNA can be more than 50 nucleotides in length.

### Multiplexing

Disclosed herein are methods, compositions, systems, and/or kits for multiplexed genome engineering. In some embodiments of the disclosure a site-directed polypeptide may comprise a guide nucleic acid, thereby forming a complex. The complex may be contacted with a target nucleic acid. The target nucleic acid may be cleaved, and/or modified by the complex. The methods, compositions, systems, and/or kits of the disclosure may be useful in modifying multiple target nucleic acids quickly, efficiently, and/or simultaneously. The method may be performed using any of the site-directed polypeptides (e.g., Cas9), guide nucleic acids, and complexes of site-directed polypeptides and guide nucleic acids as described herein.

Site-directed nucleases of the disclosure may be combined in any combination. For example, multiple CRISPR/Cas nucleases may be used to target different target sequences or different segments of the same target. In another example, Cas9 and Argonaute may be used in combination to target different targets or different sections of the same target. In some embodiments, a site-directed nuclease may be used with multiple different guide nucleic acids to target multiple different sequences simultaneously.

A nucleic acid (e.g., a guide nucleic acid) may be fused to a non-native sequence (e.g., a moiety, an endoribonuclease binding sequence, ribozyme), thereby forming a nucleic acid module. The nucleic acid module (e.g., comprising the nucleic acid fused to a non-native sequence) may be conjugated in tandem, thereby forming a multiplexed genetic targeting agent (e.g., polymodule, e.g., array). The multiplexed genetic targeting agent may comprise RNA. The multiplexed genetic targeting agent may be contacted with one or more endoribonucleases. The endoribonucleases may bind to the non-native sequence. The bound endoribonuclease may cleave a nucleic acid module of the multiplexed genetic targeting agent at a prescribed location defined by the non-native sequence. The cleavage may process (e.g., liberate) individual nucleic acid modules. In some embodiments, the processed nucleic acid modules may comprise all, some, or none, of the non-native sequence. The processed nucleic acid modules may be bound by a site-directed polypeptide, thereby forming a complex. The complex may be targeted to a target nucleic acid. The target nucleic acid may by cleaved and/or modified by the complex.

A multiplexed genetic targeting agent may be used in modifying multiple target nucleic acids at the same time, and/or in stoichiometric amounts. A multiplexed genetic targeting agent may be any nucleic acid-targeting nucleic acid as described herein in tandem. A multiplexed genetic targeting agent may refer to a continuous nucleic acid molecule comprising one or more nucleic acid modules. A nucleic acid module may comprise a nucleic acid and a non-native sequence (e.g., a moiety, endoribonuclease binding sequence, ribozyme). The nucleic acid may be non-coding RNA such as microRNA (miRNA), short interfering RNA (siRNA), long non-coding RNA (1ncRNA, or lincRNA), endogenous siRNA (endo-siRNA), piwi-interacting RNA (piRNA), trans-acting short interfering RNA (tasiRNA), repeat-associated small interfering RNA (rasiRNA), small nucleolar RNA (snoRNA), small nuclear RNA (snRNA), transfer RNA (tRNA), and ribosomal RNA (rRNA), or any combination thereof. The nucleic acid may be a coding RNA (e.g., a mRNA). The nucleic acid may be any type of RNA. In some embodiments, the nucleic acid may be a nucleic acid-targeting nucleic acid.

The non-native sequence may be located at the 3′ end of the nucleic acid module. The non-native sequence may be located at the 5′ end of the nucleic acid module. The non-native sequence may be located at both the 3′ end and the 5′ end of the nucleic acid module. The non-native sequence may comprise a sequence that may bind to a endoribonuclease (e.g., endoribonuclease binding sequence). The non-native sequence may be a sequence that is sequence-specifically recognized by an endoribonuclease (e.g., RNase T1 cleaves unpaired G bases, RNase T2 cleaves 3′ end of As, RNase U2 cleaves 3′ end of unpaired A bases). The non-native sequence may be a sequence that is structurally recognized by an endoribonuclease (e.g., hairpin structure, single-stranded-double stranded junctions, e.g., Drosha recognizes a single-stranded-double stranded junction within a hairpin). The non-native sequence may comprise a sequence that may bind to a CRISPR system endoribonuclease (e.g., Csy4, Cas5, and/or Cas6 protein).

In some embodiments, wherein the non-native sequence comprises an endoribonuclease binding sequence, the nucleic acid modules may be bound by the same endoribonuclease. The nucleic acid modules may not comprise the same endoribonuclease binding sequence. The nucleic acid modules may comprise different endoribonuclease binding sequences. The different endoribonuclease binding sequences may be bound by the same endoribonuclease. In some embodiments, the nucleic acid modules may be bound by different endoribonucleases.

The moiety may comprise a ribozyme. The ribozyme may cleave itself, thereby liberating each module of the multiplexed genetic targeting agent. Suitable ribozymes may include peptidyl transferase 23S rRNA, RnaseP, Group I introns, Group II introns, GIR1 branching ribozyme, Leadzyme, hairpin ribozymes, hammerhead ribozymes, HDV ribozymes, CPEB3 ribozymes, VS ribozymes, glmS ribozyme, CoTC ribozyme, an synthetic ribozymes.

The nucleic acids of the nucleic acid modules of the multiplexed genetic targeting agent may be identical. The nucleic acid modules may differ by 1, 2, 3, 4, 5, 6, 7, 8, 9, 10, 15, 20, 25, 30, 35, 40, 45, 50 or more nucleotides. For example, different nucleic acid modules may differ in the spacer region of the nucleic acid module, thereby targeting the nucleic acid module to a different target nucleic acid. In some instances, different nucleic acid modules may differ in the spacer region of the nucleic acid module, yet still target the same target nucleic acid. The nucleic acid modules may target the same target nucleic acid. The nucleic acid modules may target one or more target nucleic acids.

A nucleic acid module may comprise a regulatory sequence that may allow for appropriate translation or amplification of the nucleic acid module. For example, an nucleic acid module may comprise a promoter, a TATA box, an enhancer element, a transcription termination element, a ribosome-binding site, a 3′ un-translated region, a 5′ un-translated region, a 5′ cap sequence, a 3′ poly adenylation sequence, an RNA stability element, and the like.

Nucleic Acids Encoding a Designed Guide Nucleic Acid and/or Nucleic-Acid Guided Nuclease

The present disclosure provides for a nucleic acid comprising a nucleotide sequence encoding a guide nucleic acid of the disclosure, an nucleic-acid guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure. In some embodiments, a nucleic acid encoding a guide nucleic acid of the disclosure, an nucleic-acid guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be a vector (e.g., a recombinant expression vector).

In some embodiments, the recombinant expression vector may be a viral construct, (e.g., a recombinant adeno-associated virus construct), a recombinant adenoviral construct, a recombinant lentiviral construct, a recombinant retroviral construct, etc.

Suitable expression vectors may include, but are not limited to, viral vectors (e.g. viral vectors based on vaccinia virus, poliovirus, adenovirus, adeno-associated virus, SV40, herpes simplex virus, human immunodeficiency virus, a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus), plant vectors (e.g., T-DNA vector), and the like. The following vectors may be provided by way of example, for eukaryotic host cells: pXT1, pSG5, pSVK3, pBPV, pMSG, and pSVLSV40 (Pharmacia). Other vectors may be used so long as they are compatible with the host cell.

In some instances, the vector may be a linearized vector. The linearized vector may comprise a nuclease (e.g. Cas9 or Argonaute) and/or a guide nucleic acid. The linearized vector may not be a circular plasmid. The linearized vector may comprise a double-stranded break. The linearized vector may comprise a sequence encoding a fluorescent protein (e.g., orange fluorescent protein (OFP)). The linearized vector may comprise a sequence encoding an antigen (e.g., CD4). The linearized vector may be linearized (e.g., cut) in a region of the vector encoding parts of the designed nucleic acid-targeting nucleic acid. For example the linearized vector may be linearized (e.g., cut) in a 5′ region of the designed nucleic acid-targeting nucleic acid. The linearized vector may be linearized (e.g., cut) in a 3′ region of the designed nucleic acid-targeting nucleic acid. In some instances, a linearized vector or a closed supercoiled vector comprises a sequence encoding a nuclease (e.g., Cas9 or Argonaute), a promoter driving expression of the sequence encoding the nuclease (e.g., CMV promoter), a sequence encoding a marker, a sequence encoding an affinity tag, a sequence encoding portion of a guide nucleic acid, a promoter driving expression of the sequence encoding a portion of the guide nucleic acid, and a sequence encoding a selectable marker (e.g., ampicillin), or any combination thereof.

The vector may comprise a transcription and/or translation control element. Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a guide nucleic acid of the disclosure, an nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to a control element (e.g., a transcriptional control element), such as a promoter. The transcriptional control element may be functional in a eukaryotic cell, (e.g., a mammalian cell), and/or a prokaryotic cell (e.g., bacterial or archaeal cell). In some embodiments, a nucleotide sequence encoding a designed guide nucleic acid of the disclosure, a nucleic acid-guided nuclease (e.g., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to multiple control elements. Operable linkage to multiple control elements may allow expression of the nucleotide sequence encoding a guide nucleic acid of the disclosure, a nucleic acid-guided nuclease of the disclosure, an effector protein, a donor polynucleotide, a reporter element, a genetic element of interest, a component of a split system and/or any nucleic acid or proteinaceous molecule necessary to carry out the embodiments of the methods of the disclosure in either prokaryotic or eukaryotic cells.

Non-limiting examples of suitable eukaryotic promoters (i.e. promoters functional in a eukaryotic cell) may include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter may be a fungi promoter. The promoter may be a plant promoter. A database of plant promoters may be found (e.g., PlantProm). The expression vector may also contain a ribosome binding site for translation initiation and a transcription terminator. The expression vector may also include appropriate sequences for amplifying expression. The expression vector may also include nucleotide sequences encoding non-native tags (e.g., 6×His tag (SEQ ID NO: 79), hemagglutinin tag, green fluorescent protein, etc.) that are fused to the Argonaute, thus resulting in a fusion protein.

In some embodiments, a nucleotide sequence or sequences encoding a guide nucleic acid of the disclosure, a nucleic acid-guided nuclease (eg., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to an inducible promoter (e.g., heat shock promoter, tetracycline-regulated promoter, steroid-regulated promoter, metal-regulated promoter, estrogen receptor-regulated promoter, etc.). In some embodiments, a nucleotide sequence encoding a guide nucleic acid of the disclosure, a nucleic acid-guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to a constitutive promoter (e.g., CMV promoter, UBC promoter). In some embodiments, the nucleotide sequence may be operably linked to a spatially restricted and/or temporally restricted promoter (e.g., a tissue specific promoter, a cell type specific promoter, etc.).

A nucleotide sequence or sequences encoding a guide nucleic acid of the disclosure, a nucleic acid-guided nuclease (eg., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be packaged into or on the surface of biological compartments for delivery to cells. Biological compartments may include, but are not limited to, viruses (lentivirus, adenovirus), nanospheres, liposomes, quantum dots, nanoparticles, polyethylene glycol particles, hydrogels, and micelles.

Introduction of the complexes, polypeptides, and nucleic acids of the disclosure into cells may occur by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### Codon-Optimization

A polynucleotide encoding a nucleic acid-guided nuclease (eg., Cas9 or Argonaute) may be codon-optimized. This type of optimization may entail the mutation of foreign-derived (e.g., recombinant) DNA to mimic the codon preferences of the intended host organism or cell while encoding the same protein. Thus, the codons may be changed, but the encoded protein remains unchanged. For example, if the intended target cell was a human cell, a human codon-optimized polynucleotide Cas9 could be used for producing a suitable Cas9. As another non-limiting example, if the intended host cell were a mouse cell, then a mouse codon-optimized polynucleotide encoding Cas9 could be a suitable Cas9. A polynucleotide encoding a CRISPR/Cas protein may be codon optimized for many host cells of interest. A polynucleotide encoding an Argonaute may be codon optimized for many host cells of interest. A host cell may be a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Codon optimization may not be required. In some instances, codon optimization may be preferable.

### Delivery

Site-directed nucleases of the disclosure may be endogenously or recombinantly expressed within a cell. Site-directed nucleases may be encoded on a chromosome, extrachromosomally, or on a plasmid, synthetic chromosome, or artificial chromosome. Additionally or alternatively, an site-directed nucleases may be provided or delivered to the cell as a polypeptide or mRNA encoding the polypeptide. In such examples, polypeptide or mRNA may be delivered through standard mechanisms known in the art, such as through the use of cell permeable peptides, nanoparticles, viral particles, viral delivery systems, or other non-viral delivery systems.

Additionally or alternatively, guide nucleic acids may be provided by genetic or episomal DNA within a cell. Guide nucleic acids may be reverse transcribed from RNA or mRNA within a cell. Guide nucleic acids may be provided or delivered to a cell expressing a corresponding site-directed nuclease. Additionally or alternatively, guide nucleic acids may be provided or delivered concomitantly with a site-directed nuclease or sequentially. Guide nucleic acids may be chemically synthesized, assembled, or otherwise generated using standard DNA or RNA generation techniques known in the art. Additionally or alternatively, guide nucleic acids may be cleaved, released, or otherwise derived from genomic DNA, episomal DNA molecules, isolated nucleic acid molecules, or any other source of nucleic acid molecules.

### Small Molecule Inhibitors

In some embodiments, the therapeutic agent is a small-molecule inhibitor. The small molecule inhibitor may be free of a polynucleotide. The small-molecule inhibitor may be free of a peptide. In some embodiments, the small-molecule inhibitor binds directly to proteins or structures related to the expression of p16a to disrupt their functions. In general, small molecule inhibitors easily pass through a cell membrane and may not require additional modifications to assist its cellular uptake.

### Gene Targets

In some embodiments, the methods disclosed herein comprise editing a gene described herein with a CRISPR/Cas system. In some embodiments, the methods disclosed herein comprise contacting a RNA expressed from a gene described herein with an antisense oligonucleotide, thereby reducing the production of a protein encoded by the gene. In some embodiments, the methods disclosed herein describe editing a gene or modifying the expression of the gene. In some embodiments, editing the gene or modifying the expression of the gene comprises reducing the expression of the gene, reducing expression of a product of the gene (e.g. RNA, protein), reducing an activity of the product of the gene, or a combination thereof.

In some embodiments, the gene is a tumor suppressor gene. In some embodiments, the gene encodes a protein that promotes cellular senescence. In some embodiments, the gene encodes a protein that promotes cellular apoptosis. In some embodiments, the gene encodes a protein that promotes cellular differentiation. In some embodiments, the gene encodes a protein that inhibits cellular proliferation. In some embodiments, the gene encodes a protein that inhibits cell survival.

In some embodiments, the gene is characterized by a sequence having a sequence identifier (SEQ ID NO) provided herein. In some embodiments, the gene is characterized by a sequence having homology to or being homologous to a sequence identifier (SEQ ID NO) provided herein. The terms “homologous,” “homology,” or “percent homology,” when used herein to describe to an amino acid sequence or a nucleic acid sequence, relative to a reference sequence, may be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences may be determined using the most recent version of BLAST, as of the filing date of this application.

The gene may be a p16 gene. The p16 gene may be a mammalian p16 gene. The p16 gene may be a human p16 gene. The human p16 gene may have a gene accession number of NG_007485.1, although it is understood that there is natural variation in the human population. The p16 gene may comprise a p16 coding sequence. The p16 gene may comprise a p16 coding sequence of SEQ ID NO. 36. The p16 gene may be a naturally-occurring allelic variant of a p16 gene having a p16 coding sequence of SEQ ID NO. 36. The p16 coding sequence may be at least 60% homologous to SEQ ID NO. 36. The p16 coding sequence may be at least 70% homologous to SEQ ID NO. 36. p16 coding sequence may be at least 80% homologous to SEQ ID NO. 36. The p16 coding sequence may be at least 90% homologous to SEQ ID NO. 36. The p16 coding sequence may be from 60% homologous to 99% homologous to SEQ ID NO. 36. The p16 coding sequence may be from 70% homologous to 99% homologous to SEQ ID NO. 36. The p16 coding sequence may be from 80% homologous to 99% homologous to SEQ ID NO. 36. The p16 coding sequence may be from 90% homologous to 99% homologous to SEQ ID NO. 36. The p16 coding sequence may be from 95% homologous to 99% homologous to SEQ ID NO. 36.

The gene may be a Six6 gene. The Six6 gene may be a mammalian p16 gene. The Six6 gene may be a human Six6 gene. The human Six6 gene may have a gene accession number of NG_007374.2, although it is understood that there is natural variation in the human population. The Six6 gene may comprise a Six6 coding sequence. The Six6 gene may comprise a Six6 coding sequence of SEQ ID NO. 37. The Six6 gene may be a naturally-occurring allelic variant of a Six6 gene having a Six6 coding sequence of SEQ ID NO. 37. The Six6 coding sequence may be at least 60% homologous to SEQ ID NO. 37. The Six6 coding sequence may be at least 70% homologous to SEQ ID NO. 37. The Six6 coding sequence may be at least 80% homologous to SEQ ID NO. 37. The Six6 coding sequence may be at least 90% homologous to SEQ ID NO. 37. The Six6 coding sequence may be from 60% homologous to 99% homologous to SEQ ID NO. 37. The Six6 coding sequence may be from 70% homologous to 99% homologous to SEQ ID NO. 37. The Six6 coding sequence may be from 80% homologous to 99% homologous to SEQ ID NO. 37. The Six6 coding sequence may be from 90% homologous to 99% homologous to SEQ ID NO. 37. The Six6 coding sequence may be from 95% homologous to 99% homologous to SEQ ID NO. 37.

The gene may be a p53 gene. The p53 gene may be a mammalian p53 gene. The p53 gene may be a human p53 gene. The human p53 gene may have a gene accession number of NG_067013.2, although it is understood that there is natural variation in the human population. The p53 gene may comprise a p53 coding sequence. The p53 gene may comprise a p53 coding sequence of SEQ ID NO. 38. The p53 gene may be a naturally-occurring allelic variant of a p53 gene having a p53 coding sequence of SEQ ID NO. 38. The p53 coding sequence may be at least 60% homologous to SEQ ID NO. 38. The p53 coding sequence may be at least 70% homologous to SEQ ID NO. 38. The p53 coding sequence may be at least 80% homologous to SEQ ID NO. 38. The p53 coding sequence may be at least 90% homologous to SEQ ID NO. 38. The p53 coding sequence may be from 60% homologous to 99% homologous to SEQ ID NO. 38. The p53 coding sequence may be from 70% homologous to 99% homologous to SEQ ID NO. 38. The p53 coding sequence may be from 80% homologous to 99% homologous to SEQ ID NO. 38. The p53 coding sequence may be from 90% homologous to 99% homologous to SEQ ID NO. 38. The p53 coding sequence may be from 95% homologous to 99% homologous to SEQ ID NO. 38.

The gene may be an interleukin 1 (IL-1) gene. The interleukin-1 may be an interleukin 1 alpha or an interleukin 1 beta. The IL-1 gene may be a mammalian IL-1 gene. The IL-1 gene may be a human IL-1 gene. The human IL-1 gene may have a gene accession number selected from NG_008851.1 and NG_008850.1, although it is understood that there is natural variation in the human population. The IL-1 gene may comprise an IL-1 coding sequence. The IL-1 gene may comprise an IL-1 coding sequence of SEQ ID NO. 39. The IL-1 gene may be a naturally-occurring allelic variant of an IL-1 gene having a IL-1 coding sequence of SEQ ID NO. 39. The IL-1 coding sequence may be at least 60% homologous to SEQ ID NO. 39. The IL-1 coding sequence may be at least 70% homologous to SEQ ID NO. 39. The IL-1 coding sequence may be at least 80% homologous to SEQ ID NO. 39. The IL-1 coding sequence may be at least 90% homologous to SEQ ID NO. 39. The IL-1 coding sequence may be from 60% homologous to 99% homologous to SEQ ID NO. 39. The IL-1 coding sequence may be from 70% homologous to 99% homologous to SEQ ID NO. 39. The IL-1 coding sequence may be from 80% homologous to 99% homologous to SEQ ID NO. 39. The IL-1 coding sequence may be from 90% homologous to 99% homologous to SEQ ID NO. 39. The IL-1 coding sequence may be from 95% homologous to 99% homologous to SEQ ID NO. 39.

The gene may be a CDKN2D gene, encoding p19/Arf. The CDKN2D gene may be a mammalian CDKN2D gene. The CDKN2D gene may be a human CDKN2D gene. The human CDKN2D gene may have a gene accession number of NC_000019.10, although it is understood that there is natural variation in the human population. The CDKN2D gene may comprise a CDKN2D coding sequence. The CDKN2D gene may comprise a CDKN2D coding sequence of SEQ ID NO. 40. The CDKN2D gene may be a naturally-occurring allelic variant of a CDKN2D gene having a CDKN2D coding sequence of SEQ ID NO. 40. The CDKN2D coding sequence may be at least 60% homologous to SEQ ID NO. 40. The CDKN2D coding sequence may be at least 70% homologous to SEQ ID NO. 40. The CDKN2D coding sequence may be at least 80% homologous to SEQ ID NO. 40. The CDKN2D coding sequence may be at least 90% homologous to SEQ ID NO. 40. The CDKN2D coding sequence may be from 60% homologous to 99% homologous to SEQ ID NO. 40. The CDKN2D coding sequence may be from 70% homologous to 99% homologous to SEQ ID NO. 40. The CDKN2D coding sequence may be from 80% homologous to 99% homologous to SEQ ID NO. 40. The CDKN2D coding sequence may be from 90% homologous to 99% homologous to SEQ ID NO. 40. The CDKN2D coding sequence may be from 95% homologous to 99% homologous to SEQ ID NO. 40.

### Cells

Disclosed herein are methods of modifying a nucleic acid molecule expressed by a cell and cells with modified nucleic acid molecules. Further disclosed herein are methods of modifying expression and/or activity of a nucleic acid molecule expressed by a cell. In some embodiments, the methods comprise modifying the nucleic acid molecule or expression/activity thereof, wherein the nucleic acid molecule is present in a cell in vivo. In some embodiments, the methods comprise modifying the nucleic acid molecule or expression/activity thereof, wherein the nucleic acid molecule is present in a cell in vitro. In some embodiments, the methods comprise modifying the nucleic acid molecule or expression/activity thereof, wherein the nucleic acid molecule is present in a cell ex vivo. In some embodiments, the methods comprise modifying the nucleic acid molecule or expression/activity thereof, wherein the nucleic acid molecule is present in a cell in situ.

In some embodiments, the cell is a retinal cell. In some embodiments, the cell is an optic nerve cell. In some embodiments, the cell is a ganglion cell. In some embodiments, the cell is an amacrine cell. In some embodiments, the cell is a retinal ganglion cell (RGC).

In some embodiments, the cell has been isolated from the subject to be treated. In some embodiments, the cell is a stem cell. In some embodiments, the cell is a cord blood stem cell. In some embodiments, the cell is a pluripotent cell. In some embodiments, the cell is a multipotent cell. In some embodiments, the cell is an induced pluripotent stem cell (iPSC). In some embodiments, the iPSC was derived from a nerve cell. In some embodiments, the iPSC was derived from a cell of the eye. In some embodiments, the cell was an iPSC that was differentiated into a retinal ganglion cell or a multipotent progenitor thereof.

### Pharmaceutical Compositions & Modes of Administration

Disclosed herein are pharmaceutical compositions for the treatment of glaucoma, comprising therapeutic agents described herein that inhibit gene expression and protein activity.

In some embodiments, the pharmaceutical composition is a formulation for administration to the eye. In some embodiments, the formulation for administration to the eye comprises a thickening agent, surfactant, wetting agent, base ingredient, carrier, excipient or salt that makes it suitable for administration to the eye. In some embodiments, the formulation for administration to the eye has a pH, salt or tonicity that makes it suitable for administration to the eye. These aspects of formulations for administration to they eye are described herein. In some embodiments, the pharmaceutical composition is an ophthalmic preparation. The pharmaceutical composition may comprise a thickening agent in order to prolong contact time of the pharmaceutical composition and the eye. In some embodiments, the thickening agent is selected from polyvinyl alcohol, polyethylene glycol, methyl cellulose, carboxy methyl cellulose, and combinations thereof. In some embodiments, the thickening agent is filtered and sterilized.

The pharmaceutical compositions disclosed herein may comprise a pharmaceutically acceptable carrier, pharmaceutically acceptable excipient or pharmaceutically acceptable salt for the eye. Non-limiting examples of pharmaceutically acceptable carriers, pharmaceutically acceptable excipients and pharmaceutically acceptable salts for they eye, include hyaluronan, boric acid, calcium chloride, sodium perborate, phophonic acid, potassium chloride, magnesium chloride, sodium borate, sodium phosphate, and sodium chloride

The pharmaceutical compositions disclosed herein should be isotonic with lachrymal secretions. In some embodiments, the pharmaceutical composition has a tonicity from 0.5-2% NaCl. In some embodiments, the pharmaceutical composition comprises an isotonic vehicle. By way of non-limiting example, an isotonic vehicle may comprise boric acid or monobasic sodium phosphate.

In some embodiments, the pharmaceutical composition has a pH from about 3 to about 8. In some embodiments, the pharmaceutical composition has a pH from about 3 to about 7. In some embodiments, the pharmaceutical composition has a pH from about 4 to about 7. Pharmaceutical compositions outside this pH range may irritate the eye or form particulates in the eye when administered.

In some embodiments, the pharmaceutical compositions disclosed herein comprise a surfactant or wetting agent. Non-limiting examples of a surfactant employed in the pharmaceutical compositions disclosed herein are venzalkonium chloride, polysorbate 20, polysorbate 80, and dioctyl sodium sulpho succinate.

In some embodiments, the pharmaceutical compositions disclosed herein comprise a preservative that prevents microbial contamination after a container holding the pharmaceutical composition has been opened. In some embodiments, the preservative is selected from benzalkonium chloride, chlorobutanol, phenylmercuric acetate, chlorhexidine acetate, and phenylmercuric nitrate.

In some embodiments, the pharmaceutical composition (e.g., a lotion or ointment) comprises a base ingredient. The base ingredient may be selected from sodium chloride, sodium bicarbonate, boric acid, borax, zinc sulfate, a paraffin, and a wax or fatty substance. In some embodiments, the pharmaceutical composition is a lotion. In some embodiments, the lotion is provided to the subject (or a subject administering the lotion), as a powder or lyophilized product, that is reconstituted immediately before use.

Administering the pharmaceutical composition directly to the eye may avoid any undesirable off-target effects of the therapeutic agents in locations other than the eye. For example, administering the pharmaceutical composition intravenously or systemically may result in inhibiting gene expression in cells other than those of the eye, where inhibiting the gene may have deleterious effects.

In some embodiments, the pharmaceutical composition comprises a polynucleotide vector encoding any one of the nucleic acid molecules (e.g., shRNA, guide RNA, nuclease encoding polynucleotide) disclosed herein. In some embodiments, the polynucleotide vector is an expression vector. In some embodiments, the polynucleotide vector is a viral vector. In some embodiments, the pharmaceutical composition comprises a virus, wherein the virus delivers the vector and/or nucleic acid molecule to a cell of the subject. In some embodiments, the virus is a retrovirus. In some embodiments, the virus is a lentivirus. In some embodiments, the virus is an adeno-associated virus (AAV). In some embodiments, the AAV is selected from serotypes 1, 2, 5, 7, 8 and 9. In some embodiments, the AAV is AAV serotype 2. In some embodiments, the AAV is AAV serotype 8.

AAV may be particularly useful for the methods disclosed herein due to a minimal stimulation of the immune system and its ability to provide expression for years in non-dividing retinal cells. AAV may be capable of transducing multiple cell types within the retina. in some embodiments, the methods comprise intravitreal administration (e.g. injected in the vitreous humor of the eye) of AAV. in some embodiments, the methods comprise subretinal administration of AAV (e.g. injected underneath the retina).

In some embodiments, the methods and compositions disclosed herein comprise an exogenously regulatable promoter system in the AAV vector. By way of non-limiting example, the exogenously regulatable promoter system may be a tetracycline-inducible expression system.

Pharmaceutical compositions disclosed herein may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin may be exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; the MONAQUAT™ series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Compositions described herein may be formulated for controlled or sustained delivery in a manner that provides local concentration of the product (e.g., bolus, depot effect) and/or increased stability or half-life in a particular local environment. The compositions may comprise the formulation of polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents. See, for example, the description of controlled release porous polymeric microparticles for the delivery of pharmaceutical compositions in WO 93/15722.

Suitable materials for this purpose may include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals.

Formulations suitable for intramuscular, subcutaneous, peritumoral, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent may be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. The pharmaceutical composition described herein can be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

Alternatively or additionally, the compositions may be administered locally via implantation into the affected area of a membrane, sponge, or other appropriate material on to which a therapeutic agent disclosed herein has been absorbed or encapsulated. Where an implantation device is used, the device may be implanted into any suitable tissue or organ, and delivery of an inhibitor, nucleic acid, or vector disclosed herein may be directly through the device via bolus, or via continuous administration, or via catheter using continuous infusion.

Certain formulations comprising a therapeutic agent disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size.

Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one antibody of the present disclosure is administered.

In some embodiments, the pharmaceutical composition is formulated for injectable administration. In some embodiments, the methods comprise injecting the pharmaceutical composition. In some embodiments, the methods comprise administering the pharmaceutical composition in a liquid form via intraocular injection. In some embodiments, the methods comprise administering the pharmaceutical composition in a liquid form via periocular injection. In some embodiments, the methods comprise administering the pharmaceutical composition in a liquid form via intravitreal injection. While some of these modes of administration may not be appealing to the subject (e.g. intravitreal injection), they may be most effective at penetrating barriers of the eye, and the therapeutic agent may be least likely to be washed away by tears or blinking as compared to eye drops, which offer convenience and low affordability.

In some embodiments, the methods comprise administering the pharmaceutical formulation systemically. In some embodiments, the therapeutic agent is a polynucleotide vector, wherein the polynucleotide vector comprises a guide RNA, antisense oligonucleotide or Cas encoding polynucleotide. The polynucleotide vector may comprise a conditional promoter for driving expression of the nucleic acid molecules of the vector in cell-specific manner. By way of non-limiting example, the conditional promoter may drive expression only in retinal ganglion cells or only drive expression to levels that have a functional effect in retinal ganglion cells.

In some embodiments, the pharmaceutical composition is formulated for non-injectable administration. In some embodiments, the pharmaceutical composition is formulated for topical administration. By way, of non-limiting example, the nucleic acid molecule may be suspended in a saline solution or buffer that is suitable for dropping into the eye

In some embodiments, the pharmaceutical composition may be formulated as an eye drop, a gel, a lotion, an ointment, a suspension or an emulsion. In some embodiments, the pharmaceutical composition is formulated in a solid preparation such as an ocular insert. For example, the ocular insert may be formed or shaped similar to a contact lens that releases the pharmaceutical composition over a period of time, effectively conveying an extended release formulation. The gel or ointment may be applied under or inside an eyelid or in a corner of the eye.

In some embodiments, the methods may comprise administering the pharmaceutical composition immediately before sleep or before a period of time in which the subject may maintain eye closure. In some embodiments, the methods comprise instructing the subject to keep their eyes closed or administering a cover (e.g., bandage, tape, patch) to maintain eye closure for at least 1 minute, at least 5 minutes, at least 10 minutes, at least 15 minutes, at least 20 minutes, at least 30 minutes, at least 1 hour, at least 2 hours, at least 4 hours, or at least 8 hours after the pharmaceutical composition is administered. The methods may comprise instructing the subject to keep their eyes closed from 1 minute to 8 hours after the pharmaceutical composition is administered. The methods may comprise instructing the subject to keep their eyes closed from 1 minute to 2 hours after the pharmaceutical composition is administered. The methods may comprise instructing the subject to keep their eyes closed from 1 minute to 30 minutes after the pharmaceutical composition is administered.

In some embodiments, the methods comprise administering the pharmaceutical composition to the subject only once to treat glaucoma. In some embodiments, the methods comprise administering the pharmaceutical composition a first time and a second time to treat glaucoma. The first time and the second time may be separated by a period of time ranging from one hour to twelve hours. The first time and the second time may be separated by a period of time ranging from one day to one week. The first time and the second time may be separated by a period of time ranging from one week to one month. In some embodiments, the methods comprise administering the pharmaceutical composition to the subject daily, weekly, monthly, or annually. In some embodiments, the methods may comprise an aggressive treatment initially, tapering to a maintenance treatment. By way of non-limiting example, the methods may comprise initially injecting the pharmaceutical composition followed by maintaining the treatment with the pharmaceutical composition administered in the form of eye drops. Also, by way of non-limiting example, the methods may comprise initially administering weekly injections of the pharmaceutical composition from about 1 week to about 20 weeks, followed by administering the pharmaceutical composition via injection or topical administration every two to twelve months.

In some embodiments, the therapeutic agent is a small molecule inhibitor, and the pharmaceutical composition is formulated for oral administration.

### CERTAIN TERMINOLOGIES

Unless defined otherwise, all technical and scientific terms used herein have the same meaning as is commonly understood by one of skill in the art to which the claimed subject matter belongs. It is to be understood that the foregoing general description and the following examples are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. For example, “about 5 μL” means “about 5 μL” and also “5 μL.” Generally, the term “about” includes an amount that would be expected to be within experimental error. The term “about” includes values that are within 10% less to 10% greater of the value provided. For example, “about 50%” means “between 45% and 55%.” Also, by way of example, “about 30” means “between 27 and 33.”

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value. A p-value of less than 0.05 is considered statistically significant.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Alternatively, treatment is “effective” if the progression of a disease is reduced or halted. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

Using a combination of genetic association and functional studies, the examples provided herein demonstrate that SIX6 risk variant increases P16/INK4A expression and leads to RGC senescence in cell culture, animal models, and human glaucoma retinas.

In addition, these examples demonstrate that the genetic effect of the SIX6 risk-variant (rs33912345, His141Asn) is enhanced by another major POAG risk gene P16/INK4A (cyclin-dependent kinase inhibitor 2A). Upregulation of homozygous SIX6 risk alleles (CC) led to an increase in P16/INK4A expression with a subsequent cellular senescence, as evidenced in a mouse model of elevated IOP and in human POAG eyes. These data indicate that SIX6 and/or IOP promote POAG by directly increasing P16/INK4A expression, leading to RGC senescence in adult human retinas.

The examples and embodiments described herein are for illustrative purposes only and are not intended to limit the scope of the claims provided herein. Various modifications or changes suggested to persons skilled in the art are to be included within the spirit and purview of this application and scope of the appended claims.

### Example 1

### Association of SIX6-rs33912345 with Risk of POAG

POAG cases were defined as individuals for whom reliable visual field (VF) tests showed characteristic VF defects consistent with glaucomatous optic neuropathy. Individuals were classified as affected if the VF defects were reproduced on a subsequent test or if a single qualifying VF was accompanied by a cup-disc ratio (CDR) of 0.7 or more in at least one eye. The examination of the ocular anterior segment did not show signs of secondary causes for elevated IOP such as exfoliation syndrome or pigment dispersion syndrome and the filtration structures were deemed to be open based on clinical measures. Controls had normal optic nerves (cup-disc ratios≦0.6) and normal intraocular pressure.

Genetic association studies were performed and one missense variant of SIX6-rs33912345 (NM_007374.2:c.421C>A; NP_031400.2: p.His141Asn) was strongly associated with POAG. To further investigate the genetic association between SIX6-rs33912345 and POAG in Caucasian population, rs33912345 (C/A) in the SIX6 gene was genotyped using a single-nucleotide primer extension assay in 1130 POAG patients and 4036 controls. The C risk allele frequency of rs33912345 in SIX6 was significantly higher in POAG patients (0.46) than in controls (0.38), allelic P=4.49E-12, odds ratio (OR) 1.39, 95%, CI 1.27-1.53 (Table 1).

TABLE 1
Association of SIX6 with POAG

| | | | Control | | |
|---|---|---|---|---|---|
| | Risk | Case | (n = | P Value | OR |
| SNP | Allele | (n = 1,130) | 4,036) | (allelic) | (95% CI) |
| rs33912345 | C | 0.46 | 0.38 | 4.49E−12 | 1.39 |
| | | | | | (1.27-1.53) |

The primers used for genotyping are listed in Table 2. Assays were performed in triplicate. Relative mRNA levels were calculated by normalizing results using GAPDH. The primers used for qRT-PCR are listed in Table 2. The differences in quantitative PCR data were analyzed with independent two-sample t-test. SNP genotyping results were screened for deviation from Hardy-Weinberg equilibrium using Chi-squared tests. Allele association was calculated via Chi-squared test performed using the program PLINK (Purcell et al., 2007) (http://pngu.mgh.harvard.edu/purcell/plink/). Asymptomatic p-values were also generated for this test.

TABLE 2
Primer Sequence Information

| Primer name | Sequence (5′-3′) |
|---|---|
| Genotyping | |
| rs33912345 | |
| rs33912345-Forward | GTGGCCTTTCACGGTGGCAACT (SEQ ID NO. 80) |
| rs33912345-Reverse | GTTGCCCACCTGCGTAGGGGT (SEQ ID NO. 81) |
| rs33912345-Extension | GGTTAGGGTATGGATCCTGCAGGTACCACTCGCGT |
| | AGCAGGT (SEQ ID NO. 82) |
| rs1042522 | |
| rs1042522-Forward | GATGCTGTCCCCGGACGATAT (SEQ ID NO. 83) |
| rs1042522-Reverse | GCCCAGACGGAAACCGTAGCT (SEQ ID NO. 84) |
| rs1042522-Extension | CGGTGTAGGAGCTGCTGGTGCAGGGGCCACG |
| | (SEQ ID NO. 41) |
| rs3731239 | |
| rs3731239-Forward | GTAAGATGTGCTGGGACTACT (SEQ ID NO. 42) |
| rs3731239-Reverse | CGAACTCCCGACCTCAGGTGAT (SEQ ID NO. 43) |
| rs3731239-Extension | CTGTGGTGTATGTTGGAATAAATATCGAATA |
| | (SEQ ID NO. 44) |
| qPCR | |
| Human GAPDH-Forward | GAGTCAACGGATTTGGTCGT (SEQ ID NO. 45) |
| Human GAPDH-Reverse | GACAAGCTTCCCGTTCTCAG (SEQ ID NO. 46) |
| Human p16-Forward | GAGCAGCATGGAGCCTTC (SEQ ID NO. 47) |
| Human P16-Reverse | CCTCCGACCGTAACTATTCG (SEQ ID NO. 48) |
| Human SIX6-Forward | AGAATGAGTCGGTGCTACGC (SEQ ID NO. 49) |
| Human SIX6-Reverse | GCCTCCTGGTAGTTGTGCTTC (SEQ ID NO. 50) |
| Human IL6-Forward | ACTCACCTCTTCAGAACGAATTG (SEQ ID NO. 51) |
| Human IL6-Reverse | CCATCTTTGGAAGGTTCAGGTTG (SEQ ID NO. 52) |
| Mouse GAPDH-Forward | GTCAAGGCCGAGAATGGGAA (SEQ ID NO. 53) |
| Mouse GAPDH-Reverse | TTGGCTCCACCCTTCAAGTG (SEQ ID NO. 54) |
| Mouse pl6Ink4a-Forward | GCGGACTCCATGCTGCTC (SEQ ID NO. 55) |
| Mouse pl6Ink4a-Reverse | CACGACTGGGCGATTGGG (SEQ ID NO. 56) |
| Mouse Six6-Forward Mouse | ACTCCAGCAGCAGGTTCTGT (SEQ ID NO. 57) |
| Six6-Reverse Mouse | AGATGTCGCACTCACTGTCG (SEQ ID NO. 58) |
| p19Arf-Forward Mouse | CGCAGGTTCTTGGTCACTGT (SEQ ID NO. 59) |
| p19Arf-Reverse Mouse | TGTTCACGAAAGCCAGAGCG (SEQ ID NO. 60) |
| p15Cdkn2b-Forward | TTGCGGAAGGCGGAGGGAAC (SEQ ID NO. 61) |
| Mouse p15Cdkn2b-Reverse | AAGAGCAGGGCCACCGTGAC (SEQ ID NO. 62) |
| Rat P16-Forward | CGATCCGGAGCAGCATGGAGTC (SEQ ID NO. 63) |
| Rat P16-Reverse | TTCCAGCAGTGCCCGCACCTCG (SEQ ID NO. 64) |
| Rat IL6-Forward IL6 | GCCTTCTTGGGACTGATG (SEQ ID NO. 65) |
| Rat IL6-Reverse IL6 | TGTGGGTGGTATCCTCTG (SEQ ID NO. 66) |
| Rat Brn3a-Forward | CAGGAGTCCCATGTAAGA (SEQ ID NO. 67) |
| Rat Brn3a-Reverse | ACAGGGAAACACTTCTGC (SEQ ID NO. 68) |
| ChIP | |
| Human P16 promoter-Forward | ACCCTGTCCCTCAAATCC (SEQ ID NO. 69) |
| Human P16 promoter-Reverse | GGTGCCACATTCGCTAAG (SEQ ID NO. 70) |
| Human P27 promoter-Forward | CAATATGGCGGTGGAAGG (SEQ ID NO. 71) |
| Human P27 promoter-Reverse | CCGCAACCAATGGATCTC (SEQ ID NO. 72) |
| Mouse P16 promoter-Forward | ATGGAGCCCGGACTACAG (SEQ ID NO. 73) |
| Mouse P16 promoter-Reverse | GGTGTTAGCGTGGGTAGC (SEQ ID NO. 74) |
| Mouse p19Arfpromoter-Forward | CACTGTGACAAGCGAGGTGAG (SEQ ID NO. 75) |
| Mouse p19Arf promoter-Reverse | GATGGGCGTGGAGCAAAGATG (SEQ ID NO. 76) |
| Mouse p15 promoter-Forward | AAGTTGTGCCTCTGCACTC (SEQ ID NO. 77) |
| Mouse p15 promoter-Reverse | GCGATTGATGCCTCCAAAG (SEQ ID NO. 78) |

The human risk allele His141 in SIX6 is conserved across species (Table 3) and amino acid 141 is located in the homeodomain of the SIX6 protein.

TABLE 3
Conservation of SIX6 His 141 (H/N, bold) across species

| | |
|---|---|
| SIX6_H.sapiens | DGEQKTHCFKERTRHLLREWYLQDPYPNP (SEQ ID NO. 85) |
| SIX6_P.troglodytes | DGEQKTHCFKERTRHLLREWYLQDPYPNP (SEQ ID NO. 86) |
| SIX6_M.mulatta | DGEQKTHCFKERTRHLLREWYLQDPYPNP (SEQ ID NO. 87) |
| SIX6_C.lupus | DGEQKTHCFKERTRHLLREWYLQDPYPNP (SEQ ID NO. 88) |
| SIX6_M.musculus | DGEQKTHCFKERTRHLLREWYLQDPYPNP (SEQ ID NO. 89) |
| SIX6_R.norvegicus | DGEQKTHCFKERTRHLLREWYLQDPYPNP (SEQ ID NO. 90) |
| SIX6_G.gallus | DGEQKTHCFKERTRHLLREWYLQDPYPNP (SEQ ID NO. 91) |
| SIX6_D.rerio | DGEQKTHCFKERTRHLLREWYLQDPYPNP (SEQ ID NO. 92) |

A replication study was performed using a Mexican cohort with POAG and a significant association was found (P=0.11) (Table 4). A meta-analysis was then performed using these two cohorts and another cohort reported in the literature (Carnes et al., 2014). Together, the combined results indicate a significant association (allelic P=4.84E-16, Table 4).

TABLE 4
Meta-analysis results for SNP rs33912345 in POAG

| | | | Risk | Risk | | | |
|---|---|---|---|---|---|---|---|
| | Sample | Sample | Allele | Allele | | | |
| | size (n) | size (n) | Frequency | Frequency | P value | | |
| Cohorts | Case | Control | Case | Control | Allelic | OR (95% CI) | Pₕₑₜ |
| Caucasian | 1,130 | 4,036 | 0.46 | 0.38 | 4.49E−12 | 1.39 (1.27-1.53) | |
| cohort | | | | | | | |
| Mexican | 105 | 188 | 0.41 | 0.31 | 1.10E−02 | 1.57 (1.09-2.27) | |
| cohort | | | | | | | |
| Carnes | 482 | 433 | 0.47 | 0.38 | 1.05E−04 | 1.41 (1.16-1.70) | |
| cohort | | | | | | | |
| All | 1,717 | 4,657 | 0.46 | 0.38 | 4.84E−16 | 1.40 (1.29-1.52) | 0.818 |
| combined | | | | | | | |

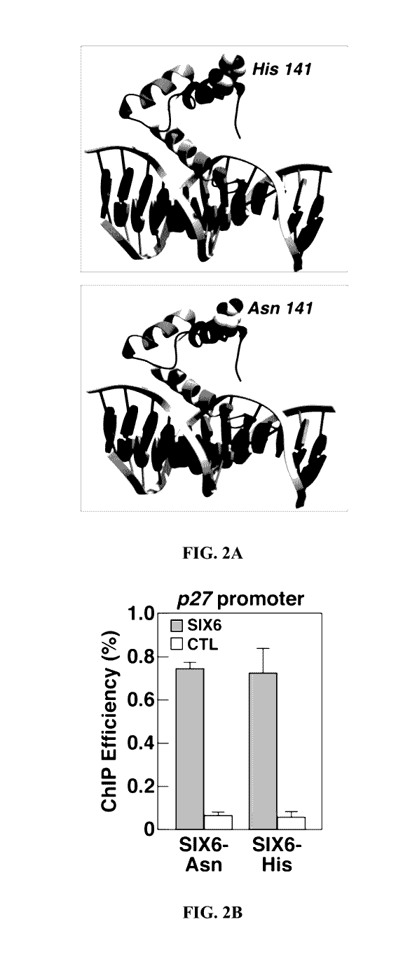

A computer modeling analysis was performed to investigate the effects of the SIX6 His141Asn variation on the three-dimensional structure and function of SIX6 with the ICM software (Cardozo et al., 1995). The SIX6 transcription factor has never been crystallized for X-ray analysis or studied by NMR. To build a structural model of the DNA-binding domain around the H141N variation, the Protein Data Bank was searched for homologous transcription factors. The search identified the following entries with fragments of related transcription factors: 2dmu, 1qry, 1vnd, 1yrn, 2lkx, and 2 solved by crystallography or NMR. Then a structural superposition of the identified domains was attempted. All had a structurally (topologically) similar DNA-binding motif, covering the area from residues around 134 to 189. The structural homology model of SIX6 was built for residues from 134 to 189 with the ICM software (Cardozo et al., 1995) by threading the SIX6 sequence onto the consensus structure. 3D modeling indicated that this residue is positioned outside of the DNA-binding surface, and thus is predicted to have no impact on DNA binding (FIG. 2A), but could rather influence the ability of SIX6 to interact with other transcription factors and co-factors.

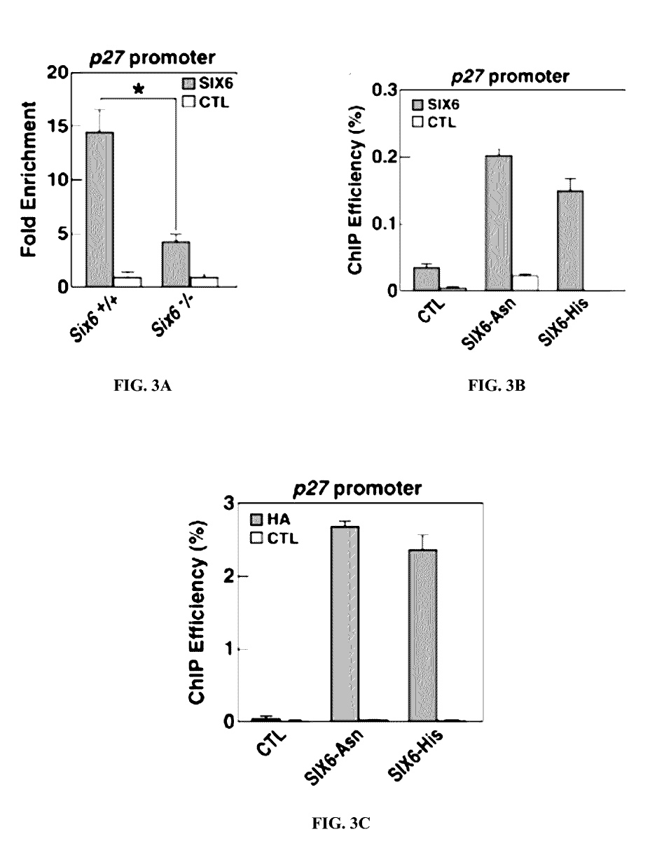

To assess both SIX6 variants in vivo for their efficiency of binding to DNA regulatory elements, the specificity of a SIX6 antibody was first tested by chromatin immunoprecipitation (ChIP) assay on chromatins isolated from wild type (WT) and SIX6 knockout (KO) retinas (cells harvested 48 h post-transfection). SIX6 protein efficiently bound to the p27 regulatory element (FIG. 3A). Using the same approach, it was observed that both the His141 and Asn141 variants of the SIX6 protein bound the p27 regulatory element in patient-derived lymphoblastoid cells with similar efficiencies (FIG. 2B). Additionally, overexpressed HA-tagged versions of SIX6 in HEK293T cells bound to the p27 regulatory element. ChIP experiments with antibodies specific to SIX6 protein or to the HA-tag demonstrated that both forms of the SIX6 protein (His141 and Asn141) bound efficiently to the p27 regulatory region (FIG. 3B,C). It was concluded that the presence of neither variant of residue 141 alters SIX6 binding efficiency to this known DNA-regulatory element.

### Example 2

### Joint Effect of SIX6 and P16/INK4A on POAG Risk

Since SIX6 and P16 were the two genes showing strongest genetic association with POAG risk, the joint effect of SIX6-rs33912345 and P16/INK4A-rs3731239 on POAG risk was investigated using a logistic regression model and calculated odds ratios (OR). Joint effects of SIX6-rs33912345 (C/A) and P16/INK4A-rs3731239 (A/G) were calculated by a logistic regression model (Chen et al., 2010; Chen et al., 2011). A global two locus (9′2) contingency table, enumerating all 9 two-locus genotype combinations, was constructed. Odds ratios and 95% confidence intervals, comparing each genotypic combination to the baseline of homozygosity for the common allele at both loci, were calculated, according to the previously described methods (Chen et al., 2010; Chen et al., 2011).

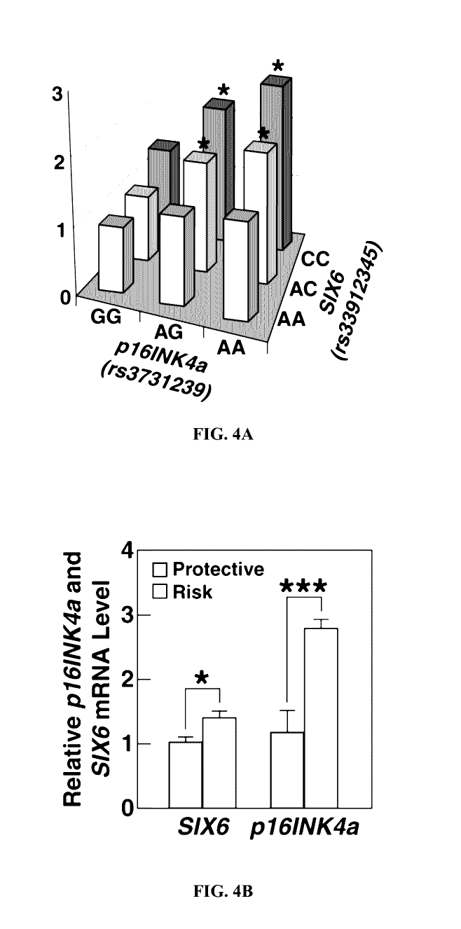

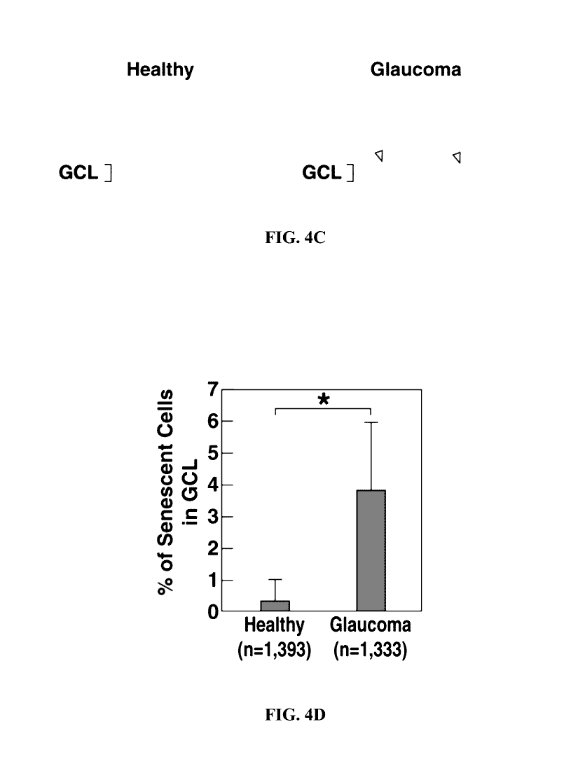

Compared to that of a baseline of non-risk alleles SIX6-rs33912345 AA and P16/INK4A-rs3731239 GG, the OR of the risk alleles SIX6-rs33912345 CC and P16/INK4A-AA was 2.73 (P<0.05, CI (1.63-4.62), FIG. 4A and Table 5, suggested their joint effect on POAG risk.

TABLE 5
Logistic regression analysis, SIX6 (rs33912345;
p16INK4a (rs3731239)

| SIX6/ | | | | |
|---|---|---|---|---|
| p16INK4a | GG | | AG | AA |
| AA | 1 | (ref) | 1.32 (0.81-2.18) | 1.41 (0.86-2.34) |
| AC | 0.99 | (0.54-1.82) | 1.73 (1.08-2.80)* | 1.99 (1.24-3.23)* |
| CC | 1.44 | (0.68-3.03) | 2.25 (1.34-3.80)* | 2.73 (1.63-4.62)* |

*p < 0.05

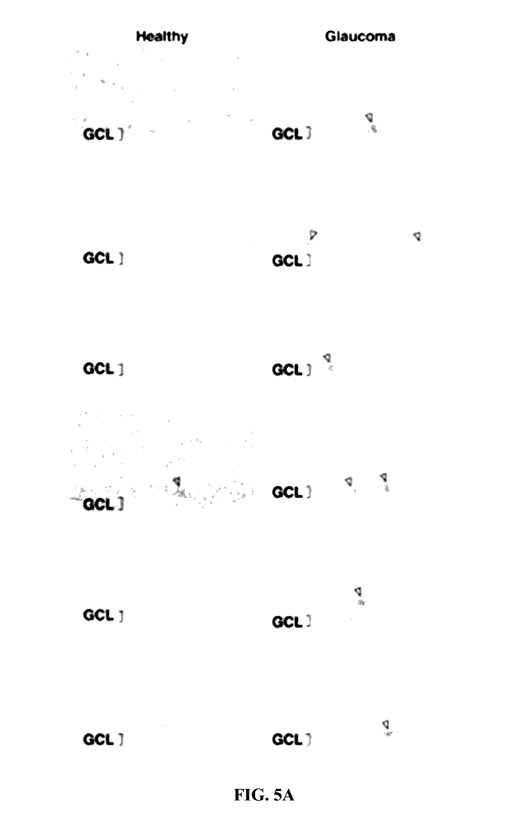

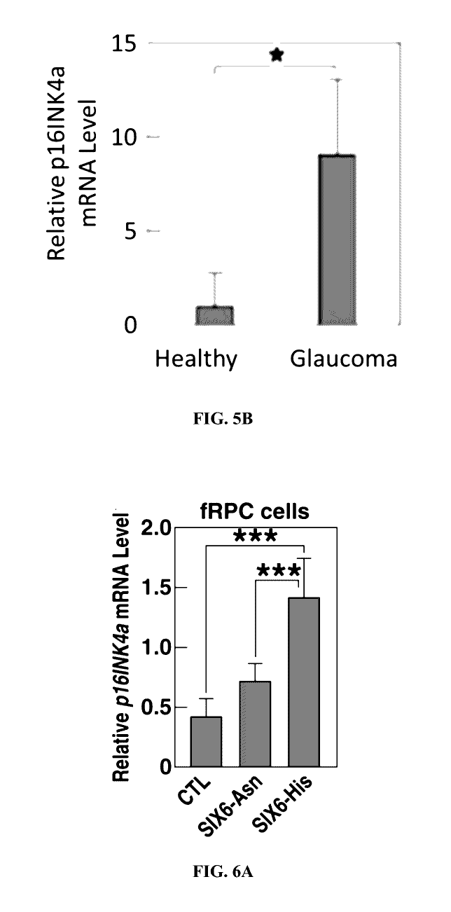

To investigate the correlation between SIX6-rs33912345 and P16/INK4A-rs3731239 genotypes and the expression of SIX6 and P16/INK4A, mRNA levels of both genes were measured in patient-derived human lymphoblastoid cells using reverse transcription followed by quantitative PCR (RT-qPCR). Higher SIX6 levels (1.4 fold) were detected in cell lines carrying the SIX6 risk allele. In addition, the expression of P16/INK4A mRNA was 2.3-fold higher in cells with risk genotype as compared to the cells with protective genotype (FIG. 4B). The levels of P16/INK4A mRNA were further measured in retinas from healthy and glaucoma patients and observed significantly elevated expression in glaucoma eyes (FIG. 5B). To investigate whether the elevated expression of P16/INK4A correlated with increased cellular senescence, senescence associated β-galactosidase (SA-βgal) assay was performed on healthy and glaucoma human retinas. Consistently, retinas from glaucoma patients exhibited elevated senescence as indicated by significantly higher number of blue-positive cells in the ganglion cell layer (GCL) (FIG. 4C, D and FIG. 5A).

### Example 3

### Transcriptional Regulation of P16/INK4A by Six6

To investigate whether SIX6 is involved in transcriptional regulation of P16/INK4A, SIX6-His141 or SIX6-Asn141 variants were transiently expressed in human fetal retinal progenitor cells (fRPCs) and P16/INK4A mRNA levels quantified using RT-qPCR.

fRPCs were isolated from human fetal neural retina at 16 weeks gestational age as previously described (Luo et al., 2014). Whole neuroretina was separated from the RPE layer, minced, and digested with collagenase I. Cells and cell clusters were plated onto human fibronectin (Akron)-coated flasks in Ultraculture Media, supplemented with 2 mM L-glutamine (Invitrogen), 10 ng/ml rhbFGF, and 20 ng/ml rhEGF in a low-oxygen incubator (37° C., 3% O2, 5% CO2, 100% humidity). Cells were passaged at 80% confluency using TrypZean, benzonase, and Defined Trypsin Inhibitor.

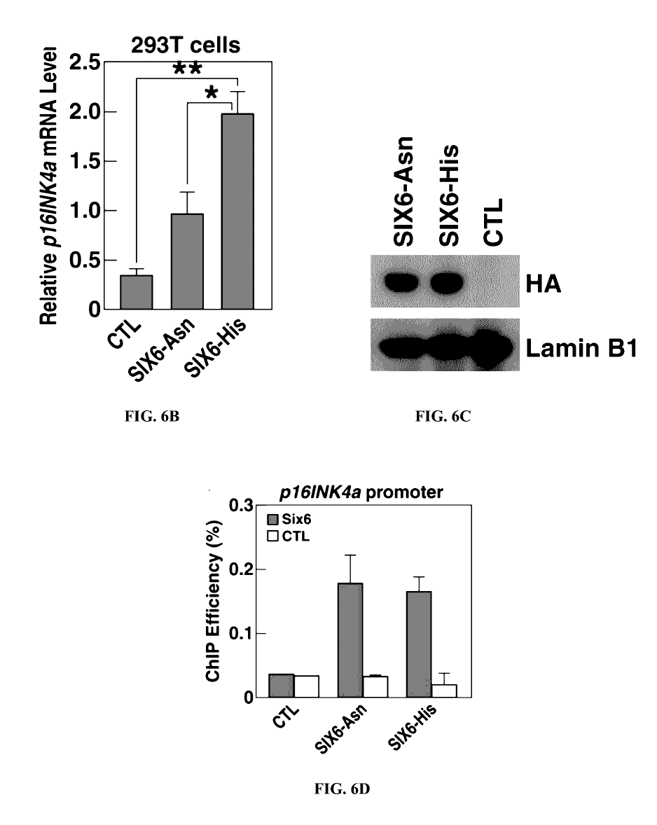

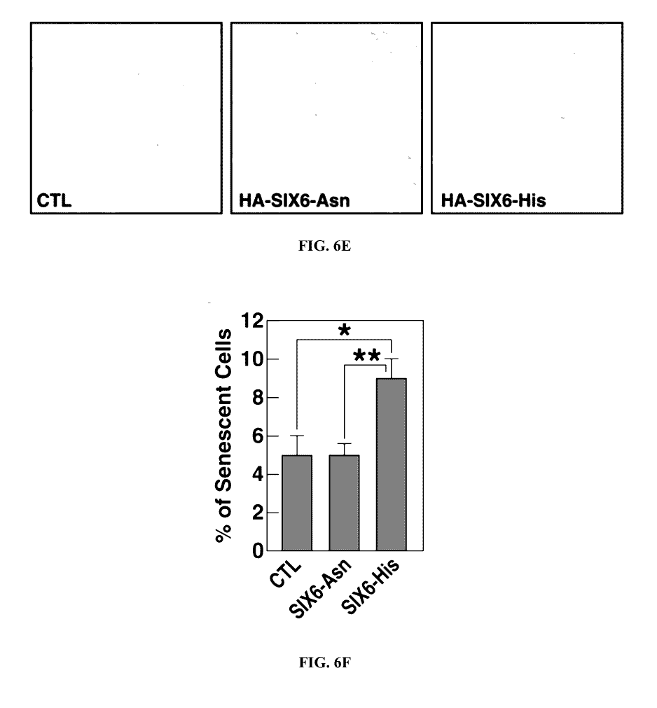

Significantly higher expression of P16/INK4A mRNA was observed in SIX6-His141 transfected cells (FIG. 6A). Similar trends were observed when SIX6-His141 and SIX6-Asn141 were overexpressed at similar levels in HEK293T cells (FIG. 6B,C), which suggested that the effect of the variation in SIX6 is not cell-type specific. To test whether both HA-tagged SIX6 protein variants can bind directly to P16/INK4A promoter, ChIP assays were performed using anti-SIX6 and anti-HA antibodies. Both protein variants bound to P16/INK4A promoter with similar efficiency (FIG. 6D and not shown). Taken together, these data suggested that SIX6 can act as a direct activator of P16/INK4A gene, and that the SIX6-His141 variant has more potential to activate P16/INK4A expression.

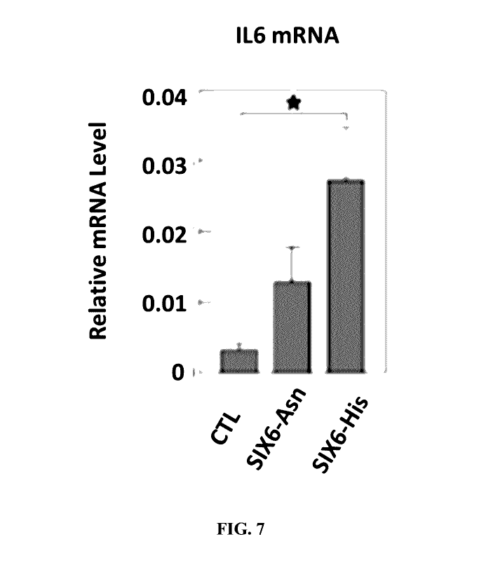

Given that elevated expression of P16/INK4A is a hallmark of cellular senescence, senescence of fRPCs by transient overexpression of either variant of SIX6 (SIX6-His141 and SIX6-Asn141) was tested. Interestingly, merely 24 h post-transfection, fRPC cells expressing SIX6-His141 variant underwent senescence twice as readily as those under control conditions or those transfected with SIX6-Asn141 variant, as assessed by a SA-β-gal assay and by upregulation of IL6, a secretory marker of senescence (FIG. 6E,F and FIG. 7). Taken together, these data indicated that SIX6-His141 risk variant increases senescence in fRPC by direct induction of P16/INK4A expression.

### Example 4

### Up-Regulation of SIX6 and P16/INK4A in a Mouse Model of Acute Glaucoma

Experimental ocular hypertension mouse models have been used extensively to study the relationship between IOP and the mechanism of glaucomatous optic neuropathy (Gross et al., 2003). To investigate the association of glaucoma in the context of SIX6-His risk variant and P16/INK4A expression, a mouse glaucoma model was used. Unilateral elevation of IOP in 1 month old C57BL/6J, p16^{-/-}, Thy1-CFP (Lindsey et al., 2013), Six6^{+/-} (Larder et al., 2011), p53^{-/-} (129-Trp53tm1Tyj/J, The Jackson Laboratory) mice was achieved through instilling the anterior chamber with saline solution and maintained at an elevated pressure of 70 mm Hg. The p16^{-/-} line was engineered to remove the common exon of E2 and E3, therefore the line is a null allele for both p16 and p19/Arf genes (Serrano et al., 1996). IOP was measured by a tonometer. To elevate IOP experimentally, animals were first anesthetized with a weight-based intraperitoneal injection of ketamine (80 mg/kg), xylazine (16 mg/kg). Additional anesthesia was provided via the same route at 45-minute intervals. Corneal anesthesia was achieved with a single drop of 0.5% proparacaine hydrochloride. A drop of 0.5% proparacaine hydrochloride and 0.5% tropicamide was then applied to the right eye. Body temperature was maintained between 37° C. and 38° C., with a water-heat pad. A 30-gauge needle was used to puncture the mid-peripheral cornea of right eye; the anterior chambers were cannulated with sterile physiologic saline (balanced salt solution;) and IOP was manometrically controlled by adjusting the saline height. IOP was monitored with an indentation tonometer (Tonolab; Icare, Espoo, Finland) and maintained at 90 mm Hg pressure for one hour. Assessment of the senescence of RGCs was made in retinal flat mounts harvested 5 days after experiments. In brief, mice were euthanized with CO₂, and eye balls were dissected and fixed in ice-cold phosphate buffered 4% paraformaldehyde, pH7.4, for 30 minutes, followed by flat mounting of the retinas.

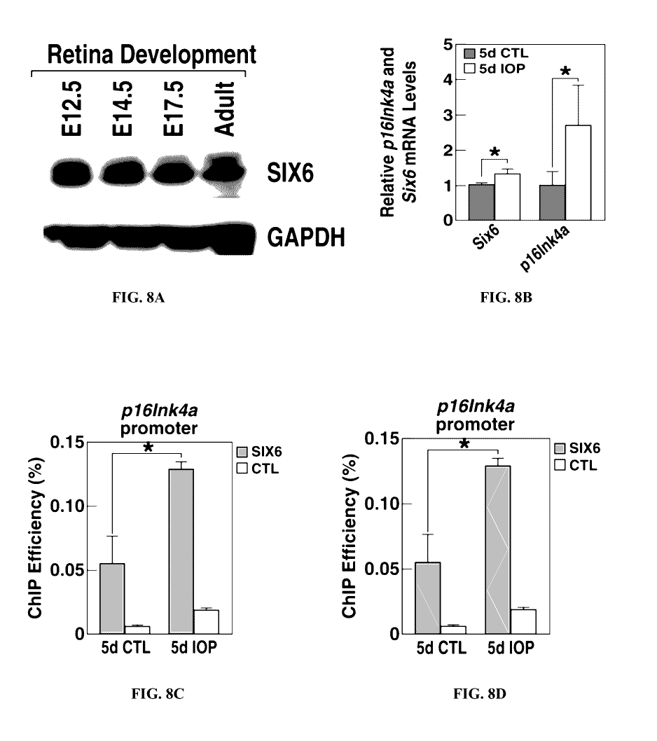

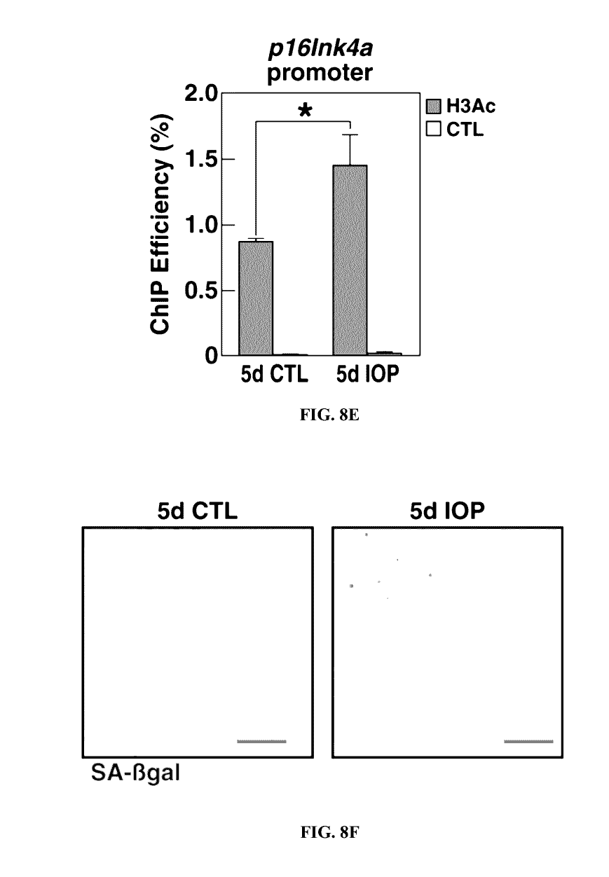

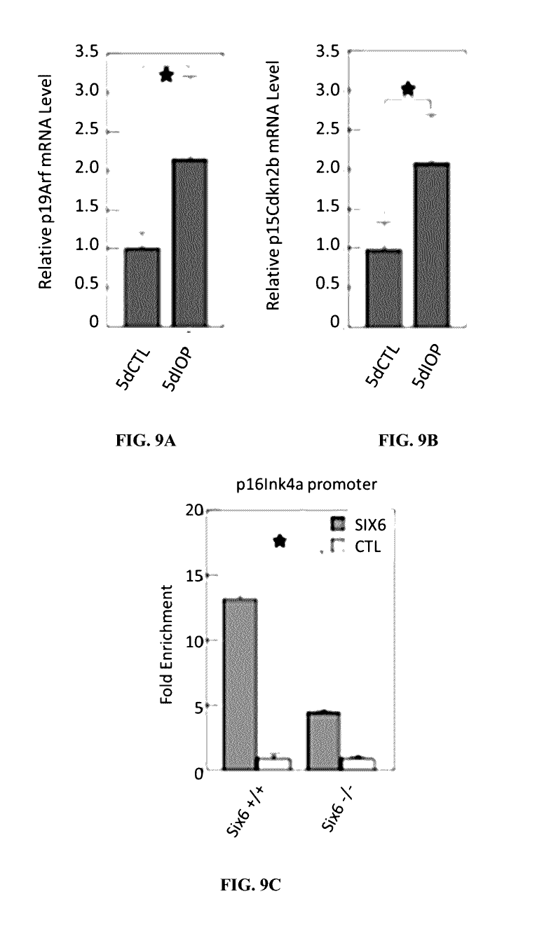

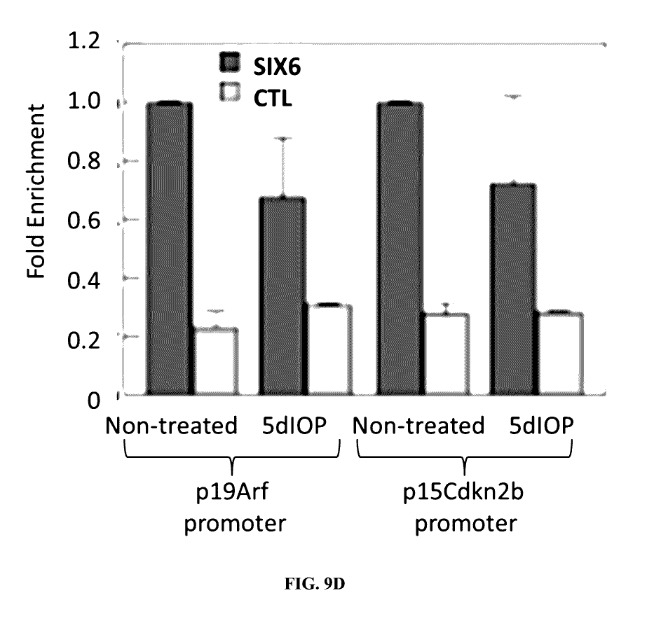

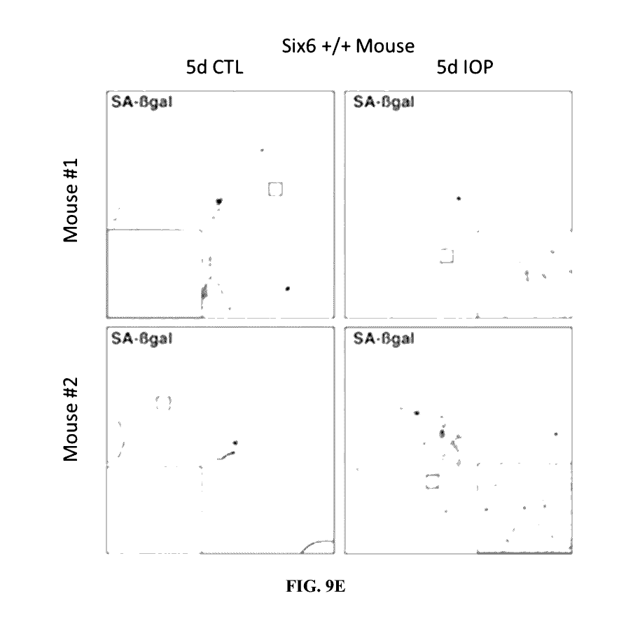

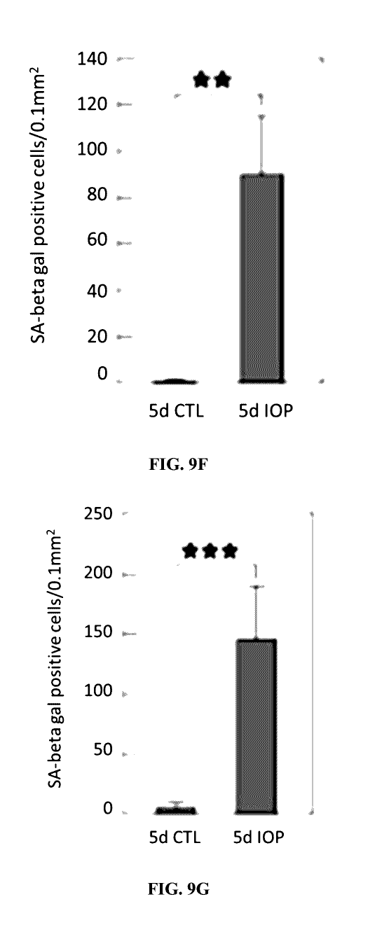

First, the sequence of mouse Six6 gene was analyzed revealing that the mouse genome encoded only the Six6-His variant (see Table 4). Western blot analysis confirmed that SIX6 was expressed in the adult retina at comparable levels to those observed in the embryonic stages (FIG. 8A). When both Six6 and P16/INK4A mRNA levels were measured 5 days post IOP elevation, both Six6 and P16/INK4A mRNA expression levels were significantly higher in retinas from IOP-treated eyes as compared to those from control eyes (FIG. 8B). Increased expression of p15/CDKN2B and p19/ARF upon IOP elevation was also noted (FIG. 9A,B).

Since SIX6 bound to P16/INK4A promoter upon engineered expression in cell culture (FIG. 6D), SIX6 binding to this promoter was also tested in adult mouse retina in vivo. To test this, ChIP efficiency in chromatins isolated from wild-type and Six6 KO retinas were compared. A detectable signal could only be observed in wild-type retinas, and not in Six6 retinas (FIG. 9C). Further, whether or not SIX6 binding to P16/INK4A promoter in the retina was altered by TOP elevation was investigated. ChIP-qPCR assay was performed in chromatins isolated from IOP-elevated and control retinas and observed that binding of SIX6 to the P16/INK4A promoter was significantly elevated upon increased IOP (FIG. 8C). This phenomenon correlated with elevated recruitment of histone acetyltransferase p300, a known co-activator of P16/INK4A expression (FIG. 8D), and increased pan-acetylation of histone H3, a modification that is associated with active regulatory elements (FIG. 8E). Importantly, there was no recruitment of Six6 to p19ARF or p15/CDKN2B promoters upon IOP elevation (FIG. 9D), suggesting that the effect of SIX6 on P16/INK4A is gene-specific. SA-β-gal assay was then used to test if the cells in the treated retinas had undergone senescence. As expected, in 5 of 5 mice tested, a dramatic accumulation of senescent cells in the IOP treated retinas was observed, as compared to only a few senescent cells observed in untreated retinas (FIGS. 8F and 9E).

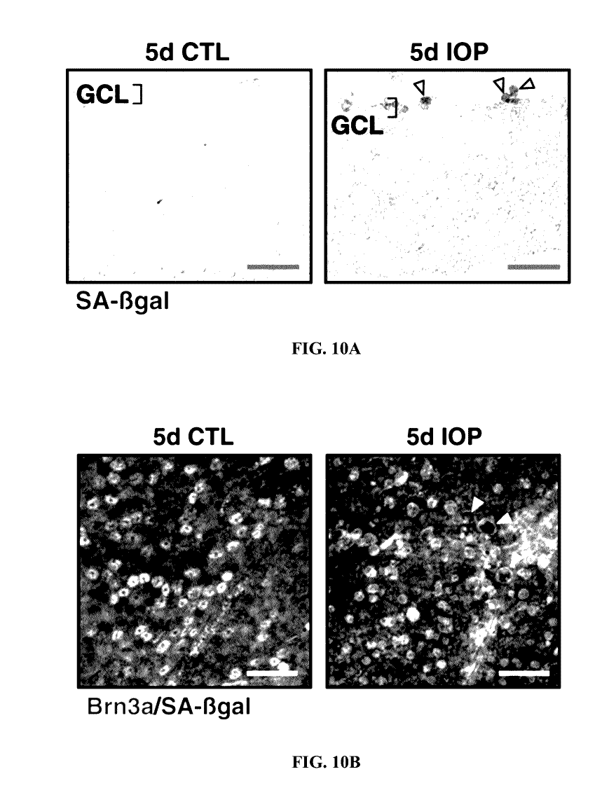

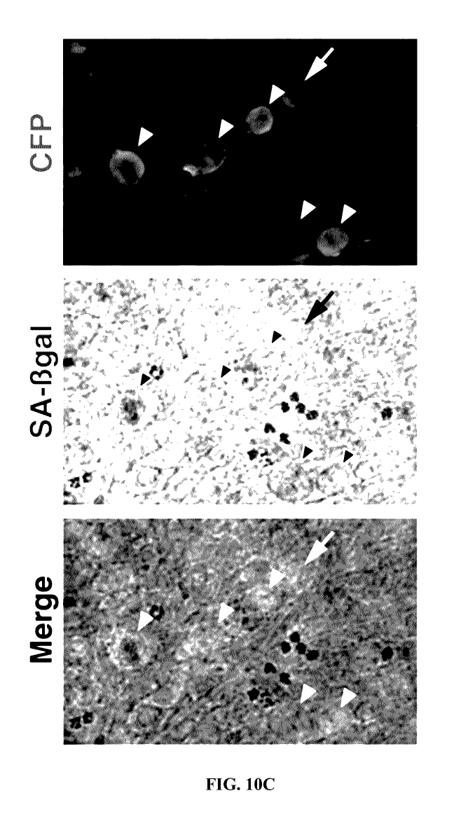

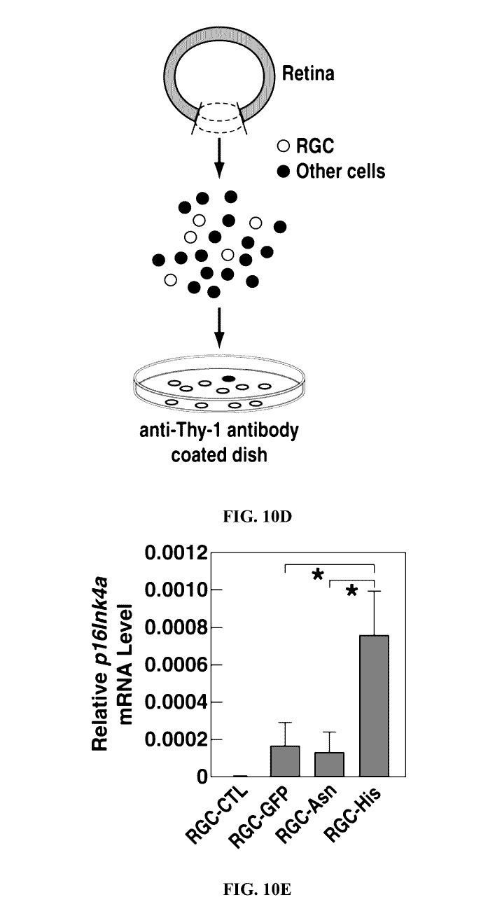

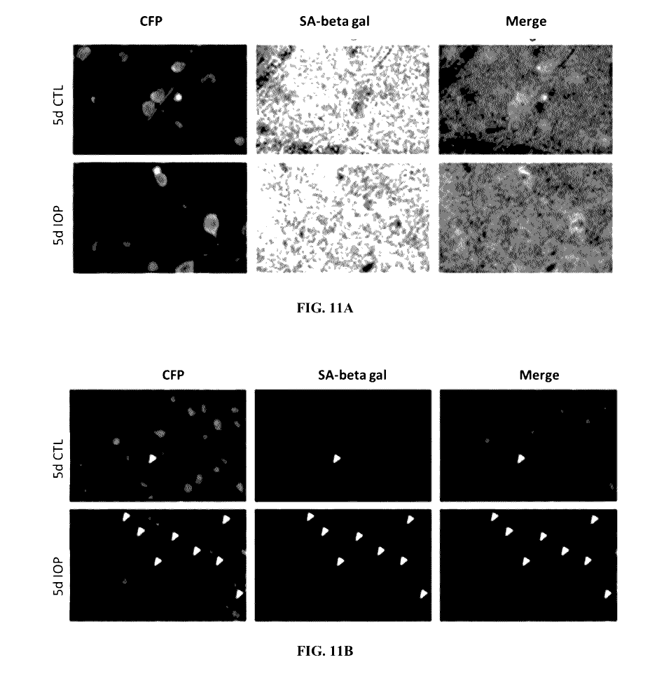

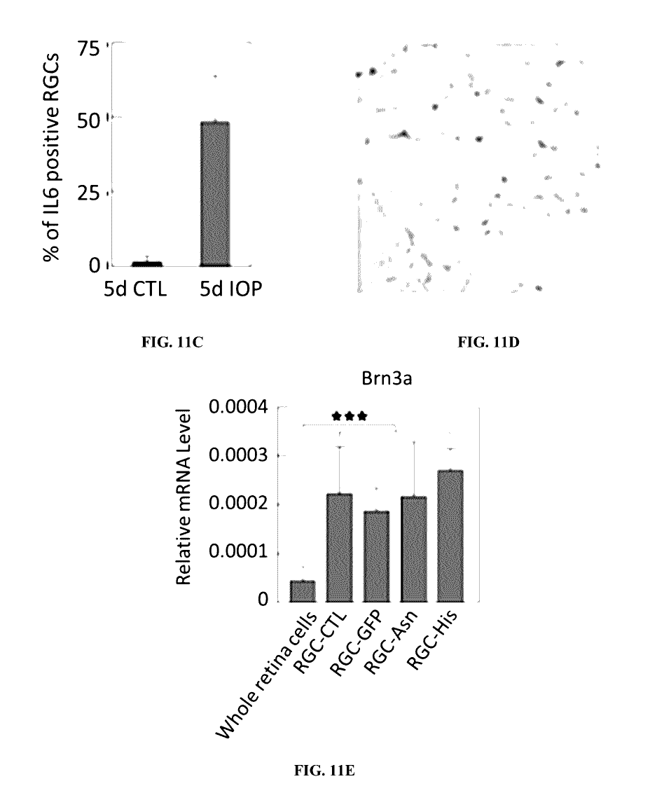

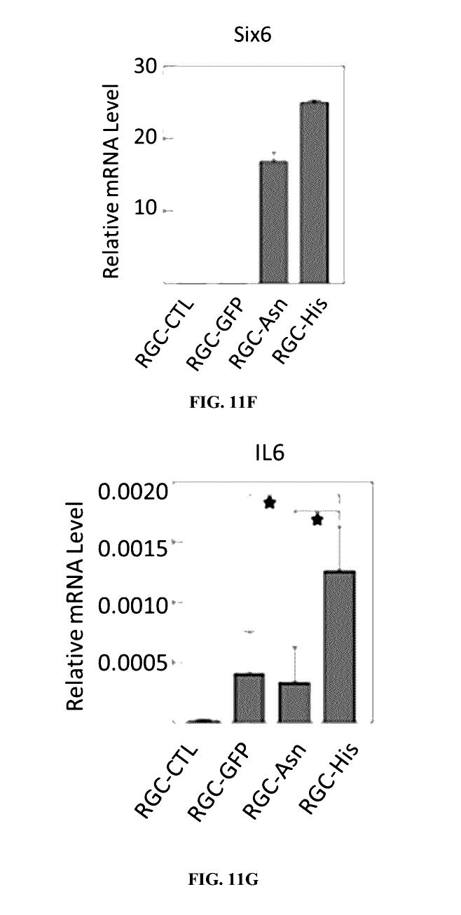

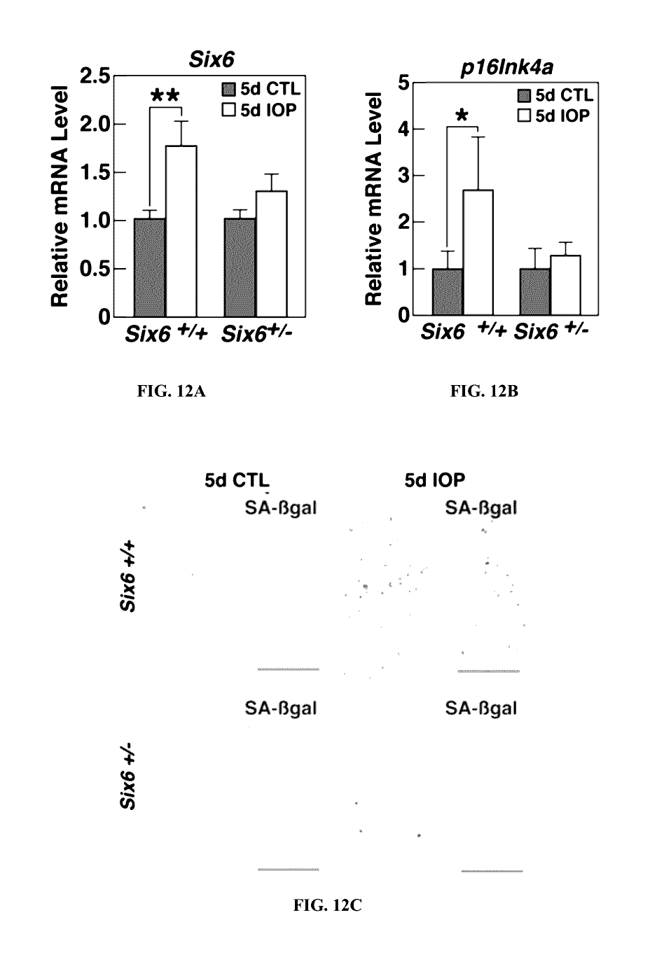

It was further investigated whether the RGCs undergo senescence in IOP treated retinas. Images of the retinal cross-sections showed that most of the senescent cells were localized in the GCL (FIG. 10A). Immunohistochemistry was performed using an anti-Brn3a antibody (an RGC marker) on IOP-treated and non-treated flat-mount retinas. Most of the β-galactosidase positive cells were also BRN3A positive (FIG. 10B). These findings were confirmed by comparing the β-gal staining pattern in Thy1-CFP transgenic mice, in which the RGCs are specifically marked by CFP fluorescence (FIG. 10C and FIG. 11A). For double staining, senescence assays were followed by anti-GFP antibody staining to reveal full fluorescence of Thy1-CFP retinas.

The effects of engineered expression of SIX6 variants on the expression of p16/INK4A and senescence associated secretory phenotype marker, IL6, in cultured RGCs was investigated. RGCs from rat retina were isolated by immunopanning using anti-Thy-1 antibody (Winzeler and Wang, 2013 provides a description of this antibody (FIG. 10D). The immunopanning procedure and retinal ganglion cell culture was performed according to the published detailed protocol (Winzeler and Wang, 2013). The efficacy of this procedure was verified by cell morphology and Brn3a expression levels in isolated RGCs as compared to whole retina cells (FIG. 11D,E). Significantly, increased expression of p16 and IL6 was observed only in the RGCs in which the His variant of Six6 was introduced (FIG. 10E, FIG. 11F,G). Consistently, there were remarkably more IL6-positive RGCs in the IOP-treated retinas than in controls (FIG. 11B,C). Taken together, these data suggested that RGCs are the primary cells affected in this glaucoma model.

### Example 5

### Lack of Either Six6 or p16 Protects Against RGC Death in Glaucoma

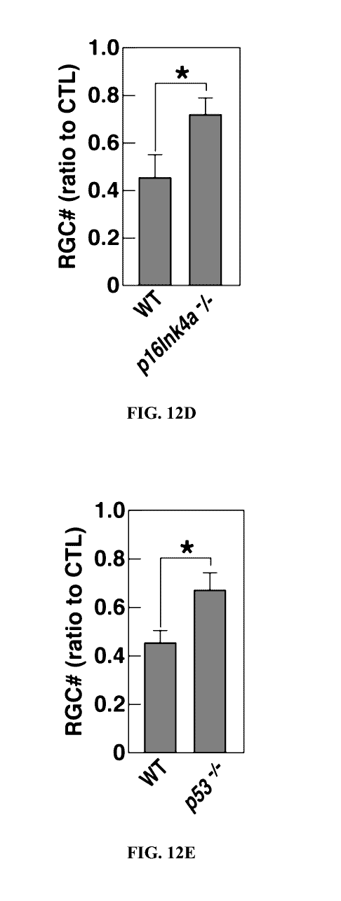

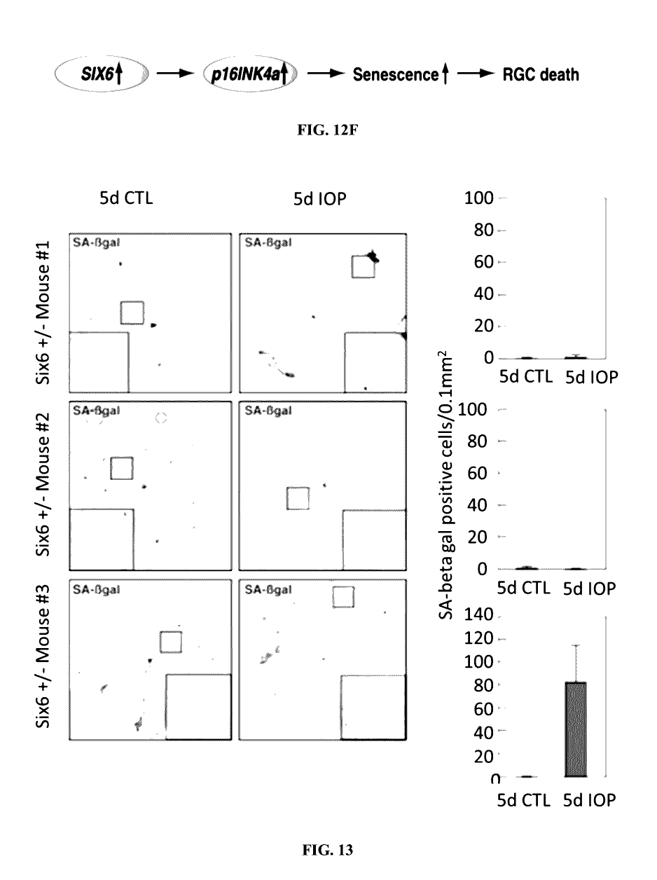

Heterozygous mice were used to test the role of SIX6 in induced senescence and in the regulation of P16/INK4A expression. As before, acute intraocular pressure elevation was applied and retinas were collected and assayed 5 days post IOP. In contrast to the wild-type littermates, reduced SIX6 expression was accompanied by decreased P16/INK4A expression upon IOP elevation in SIX6^{+/-} retinas (FIG. 12A,B). Interestingly, in SIX6^{+/-} mice, no β-gal positive cells could be observed (FIG. 12C, FIG. 13), suggesting that haploinsufficiency of SIX6 was sufficiently protective against senescence in RGCs.

Together, these results suggest a model in which increased P16/INK4A expression is a major cause of cellular senescence in glaucoma. Consistent with the above data, absence of p16/INK4a expression protected against RGC death (FIG. 12D).

To test whether the lack of P53 can prevent RGC death caused by IOP elevation, the numbers of RGCs in IOP-treated mouse retinas from p53^{-/-} mice were compared to IOP treated wild-type mice. The lack of p53 significantly attenuated RGC death upon IOP when compared to wild type mice (FIG. 12E). A genetic association of a missense variant in P53 (rs1042522 (NM_000546.5:c.215C>G; NP_000537.3: p.Pro72Arg) with POAG was also observed, consistent with its role in glaucoma pathogenesis (see Table 6).

TABLE 6
Association of p53-rs1042522 (Pro72Arg) Risk Allele and POAG

| | Risk | Case | Control | P Value | |
|---|---|---|---|---|---|
| SNP | Allele | (n = 833) | (n = 763) | (allelic) | OR (95% CI) |
| rs1042522 | C | 0.304 | 0.271 | 0.040 | 1.17 (1.00-1.37) |

Taken together, these findings led to a model (FIG. 12F) in which the IOP elevation causes the upregulation of P16/INK4A through increased expression of SIX6 and its binding (in particular the His variant) to the p16/INK4A promoter (in particular the His variant). Increased p16/INK4A expression causes RGCs to enter cellular senescence. Prolonged senescence can cause increased retinal ganglion cell death and consequent blindness.

### Example 6

### p16 Gene Editing of Retina with CRISPR/Cas

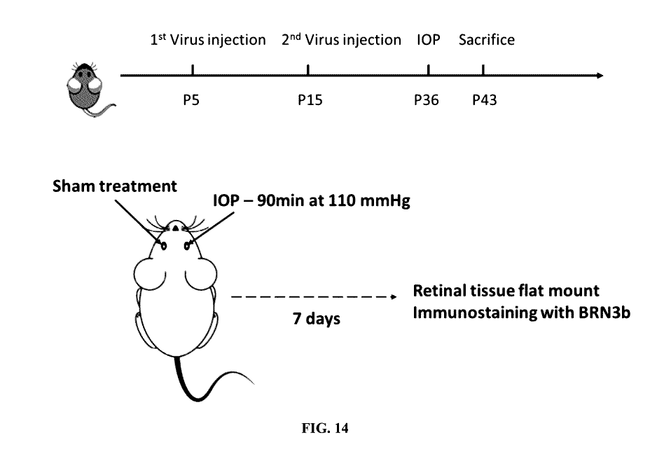

RGC quantity after elevation of intraocular pressure was compared between control mice and mice with retinal p16 gene editing. A schematic representation of treatment is shown in FIG. 14.

Plasmid Construction.

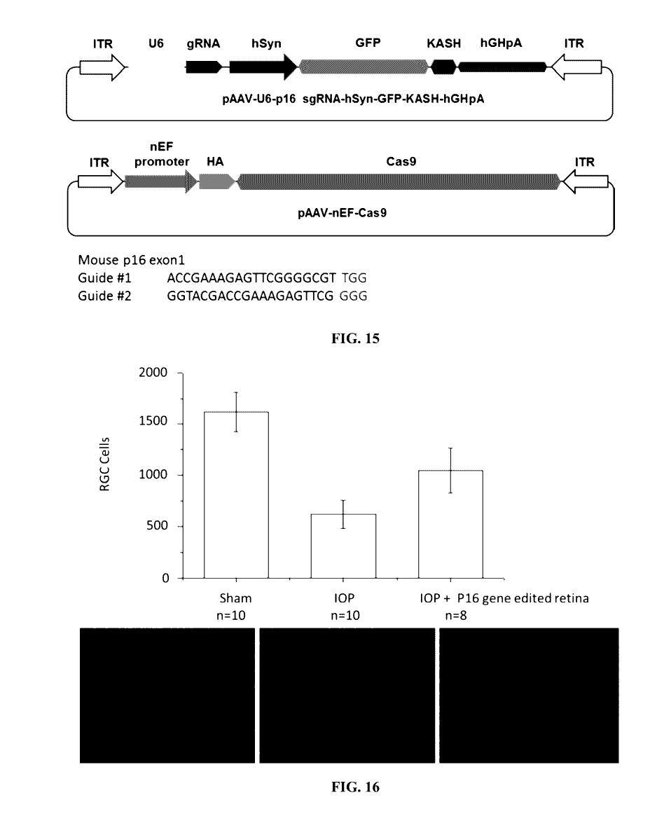

pX552 (Addgene, #60958) was used for AAV packaging and in vivo transduction (see FIG. 15). This plasmid facilitates fluorescence assisted sorting of cells a nuclei in addition to sgRNA expression. This plasmid contains two expression cassettes: EGFP-KASH and a sgRNA backbone for cloning new targeted plasmids. The plasmid can be digested with SapI creating sticky ends for ligation of annealed and phosphorylated DNA oligonucleotides designed based on the target site sequence (20 bp). Vectors were cloned by performing backbone vector digestion with SapI, an oligo phosphorylation annealing reaction to produce an sgRNA insert with overhangs corresponding to the SapI generated sticky ends of the vector, and ligation reaction of the phosphorylated/annealed oligos to the digested vector. Vectors were transformed and mini-prepped for sequencing.

sgRNA inserts were cloned into pX552 vector according to the manufacturer's instruction. Sequences for cloning G1, G2 gRNAs into pX552 vector are: pX552-G1-F:

(SEQ ID NO. 93)
ACCGCCGAAAGAGTTCGGGGCGT;
pX552-G1-R:
(SEQ ID NO. 94)
AACACGCCCCGAACTCTTTCGGC;
pX552-G2-F:
(SEQ ID NO. 95)
ACCGGTACGACCGAAAGAGTTCG;
pX552-G2-R:
(SEQ ID NO. 96)
AACCGAACTCTTTCGGTCGTACC.

Intravitreal Injections.

C57BL/6 mice at 5 days were used in the study. Mice were anesthetized with an intraperitoneal injection of a mixture of ketamine and xylazine. Pupils were dilated with 1% topical tropicamide. AAV Serotype 2/8 was used to transduce ganglion cells. For intravitreal injection, 1 μL of Cas9 and gRNAs virus mixture was injected into vitreous cavity (AAV8-Cas9: AAV8-p16 gRNA1: AAV8-p16 gRNA2=1:0.5:0.5, 1.5×10¹⁰ GC for AAV8-Cas9), or 1 μL of PBS as control. Same injection was repeated on the same animal 10 days later. The mice were kept for another 3 weeks to induce acute glaucoma.

Animal Model of Acute Glaucoma.

The mice were anesthetized by using an i.p. injection of a mixture of 100 mg/kg ketamine and 10 mg/kg xylazine. The corneas were topically anesthetized with 0.5% tetracaine hydrochloride, and the pupils were dilated with 1% tropicamide. The anterior chamber of eyes was cannulated with a 30-gauge infusion needle connected to a normal saline reservoir, which was elevated to maintain an intraocular pressure of 110 mmHg (Tono-Pen; Medtronic Solan) for 60 min. After 1 h, the needle was withdrawn, and intraocular pressure was normalized. The animals were allowed to recover for 1 week before sacrifice.

Retina Flatmount and RGC Quantification.

Mice were sacrificed and their eyes were enucleated and fixed in 4% paraformaldehyde for 1 h. After retinal flat mount preparation, RGCs were identified by using monoclonal mouse anti-Brn3a (1:200). Antibody binding was visualized by incubation with Alexa Fluor 555 donkey anti-mouse IgG (1:500). Images were obtained by using a Keyence BZ-9000 microscope. Surviving RGCs (bright red dots) were counted by using Image Pro Plus (Version 6.0; Media Cybernetics). Results are shown in FIG. 16.

The cell count in mice that received sham treatment (intraocular pressure was not elevated) was approximately 1600 cells per field. The cell count in mice with elevated IOP in the absence of p16 gene editing was approximately 600 cells per field. The cell count in mice that received IOP after p16 gene editing was approximately 1000 cells per field. The difference between cells that received IOP without gene editing and those that received IOP with gene editing was statistically significant (p<0.05). Thus, it was concluded that p16 gene editing provided an approximate 50% rescue of the effects of IOP on RGC cellular senescence.

### Example 7

### p16 siRNA, shRNA and Guide RNAs

Table 7 provides exemplary siRNA, shRNA and guide RNA targets that are used to modify p16 gene expression, as described herein. In addition, Table 7 provides target sequences in p16 useful for designing additional oligonucleotides that are used to modify p16 gene expression, as described herein.

TABLE 7
Oligonucleotides for the modification of p16 expression

| Sequence Name | Sequence |
|---|---|
| p16 human exon, positions | TCATGATGATGGGCAGCGCCCGAGTGGCGGAGCTGCT |
| 23836-24142 | GCTGCTCCACGGCGCGGAGCCCAACTGCGCCGACCCC |
| | GCCACTCTCACCCGACCCGTGCACGACGCTGCCCGGG |
| | AGGGCTTCCTGGACACGCTGGTGGTGCTGCACCGGGC |
| | CGGGGCGCGGCTGGACGTGCGCGATGCCTGGGGCCGT |
| | CTGCCCGTGGACCTGGCTGAGGAGCTGGGCCATCGCG |
| | ATGTCGCACGGTACCTGCGCGCGGCTGCGGGGGGCAC |
| | CAGAGGCAGTAACCATGCCCGCATAGATGCCGCGGAA |
| | GGTCCCTCAG (SEQ ID NO: 1) |
| p16 mouse exon 1, positions | ATGGAGTCCGCTGCAGACAGACTGGCCAGGGCGGCGG |
| 12509-12634 | CCCAGGGCCGTGTGCATGACGTGCGGGCACTGCTGGA |
| | AGCCGGGGTTTCGCCCAACGCCCCGAACTCTTTCGGTC |
| | GTACCCCGATTCAG (SEQ ID NO: 2) |
| p16 mouse exon 2, positions | TGATGATGATGGGCAACGTTCACGTAGCAGCTCTTCTG |
| 17635-17954 | CTCAACTACGGTGCAGATTCGAACTGCGAGGACCCCA |
| | CTACCTTCTCCCGCCCGGTGCACGACGCAGCGCGGGA |
| | GGCTTCCTGGACACGCTGGTGGTGCTGCACGGGTCAG |
| | GGGCTCGGCTGGATGTGCGCGATGCCTGGGGTCGCCT |
| | GCCGCTCGACTTGGCCCAAGAGCGGGGACATCAAGAC |
| | ATCGTGCGATATTTGCGTTCCGCTGGGTGCTCTTTGTG |
| | TTCCGCTGGGTGGTCTTTGTGTACCGCTGGGAACGTCG |
| | CCCAGACCGACGGGCATAG (SEQ ID NO: 3) |
| Human p1622 anti-sense | GGCGGAGCTGCTGCTGCTCCAC |
| oligonucleotide target | (SEQ ID NO: 4) |
| sequence, start position 25 | |
| Human p1622 anti-sense | GCCCGTGGACCTGGCTGAGGAG |
| oligonucleotide target | (SEQ ID NO: 5) |
| sequence, start position 187 | |
| Human p1622 anti-sense | GGCAGTAACCATGCCCGCATAG |
| oligonucleotide target | (SEQ ID NO: 6) |
| sequence, start position 263 | |
| Human p1622 anti-sense | CAGTAACCATGCCCGCATAGAT |
| oligonucleotide target | (SEQ ID NO: 7) |
| sequence, start position 265 | |
| Human p1622 anti-sense | GCCCGCATAGATGCCGCGGAAG |
| oligonucleotide target | (SEQ ID NO: 8) |
| sequence, start position 275 | |
| Human p16 shRNA, start | AGCGGAGCTGCTGCTGCTCCACTAGTGAAGCCACAGA |
| position 25 | TGTAGTGGAGCAGCAGCAGCTCCGCC (SEQ ID NO: 9) |
| Human p16 shRNA, start | ACCCGTGGACCTGGCTGAGGAGTAGTGAAGCCACAGA |
| position 187 | TGTACTCCTCAGCCAGGTCCACGGGC (SEQ ID NO: 10) |
| Human p16 shRNA, start | AGCAGTAACCATGCCCGCATAGTAGTGAAGCCACAGA |
| position 263 | TGTACTATGCGGGCATGGTTACTGCC (SEQ ID NO: 11) |
| Human p16 shRNA, start | AAGTAACCATGCCCGCATAGATTAGTGAAGCCACAGA |
| position 265 | TGTAATCTATGCGGGCATGGTTACTG (SEQ ID NO: 12) |
| Human p16 shRNA, start | ACCCGCATAGATGCCGCGGAAGTAGTGAAGCCACAGA |
| position 275 | TGTACTTCCGCGGCATCTATGCGGGC (SEQ ID NO: 13) |
| Mouse p16 shRNA #1 | CTCTGGCTTTCGTGAACATGTCGAAACATGTTCACGAA |
| | AGCCAGAG (SEQ ID NO: 14) |
| Mouse p16 shRNA #2 | GCTCTTCTGCTCAACTACGGTCGAAACCGTAGTTGAGC |
| | AGAAGAGC (SEQ ID NO: 15) |
| Mouse p16 shRNA #3 | GCTCAACTACGGTGCAGATTCCGAAGAATCTGCACCG |
| | TAGTTGAGC (SEQ ID NO: 16) |
| Human p16 guide RNA target | GGTACCGTGCGACATCGCGATGG (SEQ ID NO: 17) |
| #1 | |
| Human p16 guide RNA target | TGGGCCATCGCGATGTCGCACGG (SEQ ID NO: 18) |
| #2 | |
| Human p16 guide RNA target | ACCTTCCGCGGCATCTATGCGGG (SEQ ID NO: 19) |
| #3 | |
| Human p16 guide RNA target | TGTCGCACGGTACCTGCGCGCGG (SEQ ID NO: 20) |
| #4 | |
| Human p16 guide RNA target | CCGCGGCATCTATGCGGGCATGG (SEQ ID NO: 21) |
| #5 | |
| Human p16 guide RNA target | GACCTTCCGCGGCATCTATGCGG (SEQ ID NO: 22) |
| #6 | |
| Human p16 guide RNA target | GCCCGCATAGATGCCGCGGAAGG (SEQ ID NO: 23) |
| #7 | |
| Human p16 guide RNA target | CCATGCCCGCATAGATGCCGCGG (SEQ ID NO: 24) |
| #8 | |
| Human p16 guide RNA target | ACGGTACCTGCGCGCGGCTGCGG (SEQ ID NO: 25) |
| #9 | |
| Human p16 guide RNA target | TCCCGGGCAGCGTCGTGCACGGG (SEQ ID NO: 26) |
| #10 | |
| Mouse Exon 1 guide RNA | ACCGAAAGAGTTCGGGGCGTTGG |
| target #1 | (SEQ ID NO: 27) |
| Mouse Exon 1 guide RNA | GGTACGACCGAAAGAGTTCGGGG |
| target #2 | (SEQ ID NO: 28) |
| Mouse Exon 1 guide RNA | CGGGGCGTTGGGCGAAACCCCGG |
| target #3 | (SEQ ID NO: 29) |
| Mouse Exon 1 guide RNA | CCGAAAGAGTTCGGGGCGTTGGG |
| target #4 | (SEQ ID NO: 30) |
| Mouse Exon 1 guide RNA | GGGCCGTGTGCATGACGTGCGGG |
| target #5 | (SEQ ID NO: 31) |
| Mouse and rat common Exon | CGGTGCAGATTCGAACTGCGAGG |
| 2 guide RNA target #1 | (SEQ ID NO: 32) |
| Mouse and rat common Exon | CCCGCGCTGCGTCGTGCACCGGG |
| 2 guide RNA target #2 | (SEQ ID NO: 33) |
| Mouse and rat common Exon | CGCTGCGTCGTGCACCGGGCGGG |
| 2 guide RNA target #3 | (SEQ ID NO: 34) |
| Mouse and rat common Exon | GCGCTGCGTCGTGCACCGGGCGG |
| 2 guide RNA target #4 | (SEQ ID NO: 35) |

TABLE 8
Wildtype Human Gene Coding Sequences (exons only)

| Gene | Sequence |
|---|---|
| CDKN2A | CAAAGGGCGGCGCAGCGGCTGCCGAGCTCGGCCCTGGAGGCGGCGAG |
| (p16) | AACATGGTGCGCAGGTTCTTGGTGACCCTCCGGATTCGGCGCGCGTGC |
| | GGCCCGCCGCGAGTGAGGGTTTTCGTGGTTCACATCTCGTGGTTCACGG |
| | GGGAGTGGGCAGCGCCAGGGGCGCCCGCCGCTGTGGCCCTCGTGCTGA |
| | TGCTACTGAGGAGCCAGCGTCTAGGGCAGCAGCCGCTTCCTAGAAGAC |
| | CAGGTCATGATGATGGGCAGCGCCCGAGTGGCGGAGCTGCTGCTGCTC |
| | CACGGCGCGGAGCCCAACTGCGCCGACCCCGCCACTCTCACCCGACCC |
| | GTGCACGACGCTGCCCGGGAGGGCTTCCTGGACACGCTGGTGGTGCTG |
| | CACCGGGCCGGGGCGCGGCTGGACGTGCGCGATGCCTGGGGCCGTCTG |
| | CCCGTGGACCTGGCTGAGGAGCTGGGCCATCGCGATGTCGCACGGTAC |
| | CTGCGCGCGGCTGCGGGGGGCACCAGAGGCAGTAACCATGCCCGCATA |
| | GATGCCGCGGAAGGTCCCTCAGACATCCCCGATTGAAAGAACCAGAGA |
| | GGCTCTGAGAAACCTCCGGAAACTTAGATCATCAGTCACCGAAGGTCC |
| | TACAGGGCCACAACTGCCCCCGCCACAACCCACCCCGCTTTCGTAGTTT |
| | TCATTTAGAAAATAGAGCTTTTAAAAATGTCCTGCCTTTTAACGTAGAT |
| | ATATGCCTTCCCCCACTACCGTAAATGTCCATTTATATCATTTTTTATAT |
| | ATTCTTATAAAAATGTAAAAAAGAAAAAAAAAAAAAAAAAAAAAAA |
| | (SEQ ID NO: 36) |
| Six6 | TGGCGCACTCAGCCAGGCCCGCGGGCATCTGCTGCGTGTCCCGCTCCG |
| | GGCTCAGTGCCCTCGCCGCCGCCGGCACTGCCTCGATGTTCCAGCTGCC |
| | CATCTTGAATTTCAGCCCCCAGCAAGTGGCCGGGGTATGTGAGACCCT |
| | GGAAGAGAGCGGCGATGTGGAGCGCCTGGGTCGCTTCCTCTGGTCGCT |
| | GCCCGTGGCCCCTGCGGCCTGCGAGGCCCTCAACAAGAATGAGTCGGT |
| | GCTACGCGCACGAGCCATCGTGGCCTTTCACGGTGGCAACTACCGCGA |
| | GCTCTATCATATCCTGGAAAACCACAAGTTCACCAAGGAGTCGCACGC |
| | CAAGCTGCAGGCGCTGTGGCTTGAAGCACACTACCAGGAGGCTGAGAA |
| | GCTGCGTGGAAGACCCCTGGGACCTGTGGACAAGTACCGAGTAAGGAA |
| | GAAGTTCCCGCTGCCGCGCACCATTTGGGACGGCGAACAGAAGACACA |
| | CTGCTTCAAGGAGCGCACGCGGAACCTGCTACGCGAGTGGTACCTGCA |
| | GGATCCATACCCTAACCCCAGCAAAAAACGTGAGCTCGCCCAGGCAAC |
| | CGGACTGACCCCTACGCAGGTGGGCAACTGGTTCAAAAACCGCCGACA |
| | AAGGGACCGAGCGGCTGCAGCCAAGAACAGACTCCAGCAGCAGGTCC |
| | TGTCACAGGGTTCCGGGCGGGCACTACGGGCGGAGGGCGACGGCACG |
| | CCAGAGGTGCTGGGCGTCGCCACCAGCCCGGCCGCCAGTCTATCCAGC |
| | AAGGCGGCCACTTCAGCCATCTCCATCACGTCCAGCGACAGCGAGTGC |
| | GACATCTGAGTTGCCCATCCAGGATGCTCAGAAGCAGATTCCAGTGTA |
| | AAAACGAGAAAAACAAAATGAAAGAGGGGAAGAAGATGAGAGACCT |
| | GCAA (SEQ ID NO: 37) |
| p53 | CCAGGGAGCAGGTAGCTGCTGGGCTCCGGGGACACTTTGCGTTCGGGC |
| | TGGGAGCGTGCTTTCCACGACGGTGACACGCTTCCCTGGATTGGCAGC |
| | CAGACTGCCTTCCGGGTCACTGCCATGGAGGAGCCGCAGTCAGATCCT |
| | AGCGTCGAGCCCCCTCTGAGTCAGGAAACATTTTCAGACCTATGGAAA |
| | CTACTTCCTGAAAACAACGTTCTGTCCCCCTTGCCGTCCCAAGCAATGG |
| | ATGATTTGATGCTGTCCCCGGACGATATTGAACAATGGTTCACTGAAG |
| | ACCCAGGTCCAGATGAAGCTCCCAGAATGCCAGAGGCTGCTCCCCGCG |
| | TGGCCCCTGCACCAGCAGCTCCTACACCGGCGGCCCCTGCACCAGCCC |
| | CCTCCTGGCCCCTGTCATCTTCTGTCCCTTCCCAGAAAACCTACCAGGG |
| | CAGCTACGGTTTCCGTCTGGGCTTCTTGCATTCTGGGACAGCCAAGTCT |
| | GTGACTTGCACGTACTCCCCTGCCCTCAACAAGATGTTTTGCCAACTGG |
| | CCAAGACCTGCCCTGTGCAGCTGTGGGTTGATTCCACACCCCCGCCCGG |
| | CACCCGCGTCCGCGCCATGGCCATCTACAAGCAGTCACAGCACATGAC |
| | GGAGGTTGTGAGGCGCTGCCCCCACCATGAGCGCTGCTCAGATAGCGA |
| | TGGTCTGGCCCCTCCTCAGCATCTTATCCGAGTGGAAGGAAATTTGCGT |
| | GTGGAGTATTTGGATGACAGAAACACTTTTCGACATAGTGTGGTGGTG |
| | CCCTATGAGCCGCCTGAGGTTGGCTCTGACTGTACCACCATCCACTACA |
| | ACTACATGTGTAACAGTTCCTGCATGGGCGGCATGAACCGGAGGCCCA |
| | TCCTCACCATCATCACACTGGAAGACTCCAGTGGTAATCTACTGGGAC |
| | GGAACAGCTTTGAGGTGCGTGTTTGTGCCTGTGCTGGGAGAGACCGGC |
| | GCACAGAGGAAGAGAATCTCCGCAAGAAAGGGGAGCCTCACCACGAG |
| | CTGCCCCCAGGGAGCACTAAGCGAGCACTGCCCAACAACACCAGCTCC |
| | TCTCCCCAGCCAAAGAAGAAACCACTGGATGGAGAATATTTCACCCTT |
| | CAGATCCGTGGGCGTGAGCGCTTCGAGATGTTCCGAGAGCTGAATGAG |
| | GCCTTGGAACTCAAGGATGCCCAGGCTGGGAAGGAGCCAGGGGGGAG |
| | CAGGGCTCACTCCAGCCACCTGAAGTCCAAAAAGGGTCAGTCTACCTC |
| | CCGCCATAAAAAACTCATGTTCAAGACAGAAGGGCCTGACTCAGACTG |
| | ACATTCTCCACTTCTTGTTCCCCACTGACAGCCTCCCACCCCCATCTCTC |
| | CCTCCCCTGCCATTTTGGGTTTTGGGTCTTTGAACCCTTGCTTGCAATAG |
| | GTGTGCGTCAGAAGCACCCAGGACTTCCATTTGCTTTGTCCCGGGGCTC |
| | CACTGAACAAGTTGGCCTGCACTGGTGTTTTGTTGTGGGGAGGAGGAT |
| | GGGGAGTAGGACATACCAGCTTAGATTTTAAGGTTTTTACTGTGAGGG |
| | ATGTTTGGGAGATGTAAGAAATGTTCTTGCAGTTAAGGGTTAGTTTACA |
| | ATCAGCCACATTCTAGGTAGGGGCCCACTTCACCGTACTAACCAGGGA |
| | AGCTGTCCCTCACTGTTGAATTTTCTCTAACTTCAAGGCCCATATCTGT |
| | GAAATGCTGGCATTTGCACCTACCTCACAGAGTGCATTGTGAGGGTTA |
| | ATGAAATAATGTACATCTGGCCTTGAAACCACCTTTTATTACATGGGGT |
| | CTAGAACTTGACCCCCTTGAGGGTGCTTGTTCCCTCTCCCTGTTGGTCG |
| | GTGGGTTGGTAGTTTCTACAGTTGGGCAGCTGGTTAGGTAGAGGGAGT |
| | TGTCAAGTCTCTGCTGGCCCAGCCAAACCCTGTCTGACAACCTCTTGGT |
| | GAACCTTAGTACCTAAAAGGAAATCTCACCCCATCCCACACCCTGGAG |
| | GATTTCATCTCTTGTATATGATGATCTGGATCCACCAAGACTTGTTTTAT |
| | GCTCAGGGTCAATTTCTTTTTTCTTTTTTTTTTTTTTTTTCTTTTTCTTTGA |
| | GACTGGGTCTCGCTTTGTTGCCCAGGCTGGAGTGGAGTGGCGTGATCTT |
| | GGCTTACTGCAGCCTTTGCCTCCCCGGCTCGAGCAGTCCTGCCTCAGCC |
| | TCCGGAGTAGCTGGGACCACAGGTTCATGCCACCATGGCCAGCCAACT |
| | TTTGCATGTTTTGTAGAGATGGGGTCTCACAGTGTTGCCCAGGCTGGTC |
| | TCAAACTCCTGGGCTCAGGCGATCCACCTGTCTCAGCCTCCCAGAGTGC |
| | TGGGATTACAATTGTGAGCCACCACGTCCAGCTGGAAGGGTCAACATC |
| | TTTTACATTCTGCAAGCACATCTGCATTTTCACCCCACCCTTCCCCTCCT |
| | TCTCCCTTTTTATATCCCATTTTTATATCGATCTCTTATTTTACAATAAA |
| | ACTTTGCTGCCAAAAAAAAAAAAAAAAAAAA (SEQ ID NO: 38) |
| Inter- | ACCAAACCTCTTCGAGGCACAAGGCACAACAGGCTGCTCTGGGATTCT |
| leukin 1 | CTTCAGCCAATCTTCATTGCTCAAGTGTCTGAAGCAGCCATGGCAGAA |
| | GTACCTGAGCTCGCCAGTGAAATGATGGCTTATTACAGTGGCAATGAG |
| | GATGACTTGTTCTTTGAAGCTGATGGCCCTAAACAGATGAAGTGCTCCT |
| | TCCAGGACCTGGACCTCTGCCCTCTGGATGGCGGCATCCAGCTACGAA |
| | TCTCCGACCACCACTACAGCAAGGGCTTCAGGCAGGCCGCGTCAGTTG |
| | TTGTGGCCATGGACAAGCTGAGGAAGATGCTGGTTCCCTGCCCACAGA |
| | CCTTCCAGGAGAATGACCTGAGCACCTTCTTTCCCTTCATCTTTGAAGA |
| | AGAACCTATCTTCTTCGACACATGGGATAACGAGGCTTATGTGCACGA |
| | TGCACCTGTACGATCACTGAACTGCACGCTCCGGGACTCACAGCAAAA |
| | AAGCTTGGTGATGTCTGGTCCATATGAACTGAAAGCTCTCCACCTCCAG |
| | GGACAGGATATGGAGCAACAAGTGGTGTTCTCCATGTCCTTTGTACAA |
| | GGAGAAGAAAGTAATGACAAAATACCTGTGGCCTTGGGCCTCAAGGA |
| | AAAGAATCTGTACCTGTCCTGCGTGTTGAAAGATGATAAGCCCACTCT |
| | ACAGCTGGAGAGTGTAGATCCCAAAAATTACCCAAAGAAGAAGATGG |
| | AAAAGCGATTTGTCTTCAACAAGATAGAAATCAATAACAAGCTGGAAT |
| | TTGAGTCTGCCCAGTTCCCCAACTGGTACATCAGCACCTCTCAAGCAGA |
| | AAACATGCCCGTCTTCCTGGGAGGGACCAAAGGCGGCCAGGATATAAC |
| | TGACTTCACCATGCAATTTGTGTCTTCCTAAAGAGAGCTGTACCCAGAG |
| | AGTCCTGTGCTGAATGTGGACTCAATCCCTAGGGCTGGCAGAAAGGGA |
| | ACAGAAAGGTTTTTGAGTACGGCTATAGCCTGGACTTTCCTGTTGTCTA |
| | CACCAATGCCCAACTGCCTGCCTTAGGGTAGTGCTAAGAGGATCTCCT |
| | GTCCATCAGCCAGGACAGTCAGCTCTCTCCTTTCAGGGCCAATCCCCAG |
| | CCCTTTTGTTGAGCCAGGCCTCTCTCACCTCTCCTACTCACTTAAAGCCC |
| | GCCTGACAGAAACCACGGCCACATTTGGTTCTAAGAAACCCTCTGTCA |
| | TTCGCTCCCACATTCTGATGAGCAACCGCTTCCCTATTTATTTATTTATT |
| | TGTTTGTTTGTTTTATTCATTGGTCTAATTTATTCAAAGGGGGCAAGAA |
| | GTAGCAGTGTCTGTAAAAGAGCCTAGTTTTTAATAGCTATGGAATCAAT |
| | TCAATTTGGACTGGTGTGCTCTCTTTAAATCAAGTCCTTTAATTAAGAC |
| | TGAAAATATATAAGCTCAGATTATTTAAATGGGAATATTTATAAATGA |
| | GCAAATATCATACTGTTCAATGGTTCTGAAATAAACTTCACTGAAAAA |
| | AAAAAAAAAAAAAAAAAAAAAA (SEQ ID NO: 39) |
| CDKN2D | ATGCTGCTGGAGGAGGTTCGCGCCGGCGACCGGCTGAGTGGGGCGGCG |
| (P19) | GCCCGGGGCGACGTGCAGGAGGTGCGCCGCCTTCTGCACCGCGAGCTG |
| | GTGCATCCCGACGCCCTCAACCGCTTCGGCAAGACGGCGCTGCAGGTC |
| | ATGATGTTTGGCAGCACCGCCATCGCCCTGGAGCTGCTGAAGCAAGGT |
| | GCCAGCCCCAATGTCCAGGACACCTCCGGTACCAGTCCAGTCCATGAC |
| | GCAGCCCGCACTGGATTCCTGGACACCCTGAAGGTCCTAGTGGAGCAC |
| | GGGGCTGATGTCAACGTGCCTGATGGCACCGGGGCACTTCCAATCCAT |
| | CTGGCAGTTCAAGAGGGTCACACTGCTGTGGTCAGCTTTCTGGCAGCTG |
| | AATCTGATCTCCATCGCAGGGACGCCAGGGGTCTCACACCCTTGGAGC |
| | TGGCACTGCAGAGAGGGGCTCAGGACCTCGTGGACATCCTGCAGGGCC |
| | ACATGGTGGCCCCGCTG (SEQ ID NO: 40) |

What is claimed is:

1. A method of treating a subject for glaucoma or symptoms thereof comprising administering to an eye of the subject:
a) a guide RNA that hybridizes to a target site of a gene, wherein the gene encodes a protein that contributes to glaucoma or symptoms thereof; and
b) a Cas nuclease that cleaves a strand of the gene at the target site,
wherein cleaving the strand modifies expression of the gene, thereby reducing contribution of the protein to glaucoma or symptoms thereof.

2. The method of claim 1, comprising administering a repair template to replace a portion of the gene.

3. The method of claim 1, comprising reducing expression of the protein or reducing activity of the protein.

4. The method of claim 1, wherein the method results in reducing retinal ganglion cell senescence in the eye.

5. The method of claim 1, comprising administering a polynucleotide encoding the Cas nuclease and the guide RNA in a delivery vehicle selected from a vector, a liposome, and a ribonucleoprotein.

6. The method of claim 1, wherein the gene is a p16 gene.

7. The method of claim 1, wherein the subject harbors a p16 allelic variant of a wildtype p16 gene, wherein the wildtype p16 gene comprises a coding sequence of SEQ ID NO. 36.

8. The method of claim 7, wherein the p16 allelic variant harbors a single nucleotide polymorphism that contributes to glaucoma or symptoms thereof.

9. The method of claim 8, wherein the single nucleotide polymorphism is an alanine residue at rs1042522.

10. The method of claim 6, wherein the guide RNA targets the Cas nuclease to a sequence of the p16 gene selected from SEQ ID NOS: 17-35.

11. The method of claim 1, wherein the gene is a Six6 gene.

12. The method of claim 1, wherein the subject harbors a p16 allelic variant of a wildtype p16 gene, wherein the wildtype p16 gene comprises a coding sequence of SEQ ID NO. 37.

13. The method of claim 12, wherein the Six6 gene comprises a single nucleotide polymorphism of a cytosine at rs33912345.

14. A method of treating a subject for glaucoma comprising administering to an eye of the subject an antisense oligonucleotide that hybridizes to a p16 messenger RNA, thereby reducing expression of the p16 gene via RNA interference.

15. The method of claim 14, wherein reducing expression of the p16 gene reduces retinal ganglion cell senescence.

16. The method of claim 15, wherein retinal ganglion cell senescence is reduced from about 10% to about 90%.

17. The method of claim 15, wherein retinal ganglion cell senescence is reduced at least about 40%.

18. The method of claim 14, wherein the antisense oligonucleotide is a short hairpin RNA encoded by a sequence selected from SEQ ID NOS: 9-13.

19. The method of claim 14, wherein the antisense oligonucleotide is administered in a polynucleotide vector, a liposome, or ribonucleoprotein.

20. A pharmaceutical composition for the treatment of glaucoma comprising:
a. a polynucleotide encoding a Cas protein; and
b. a guide RNA that is complementary to a portion of a gene selected from a p16 gene and a Six6 gene.

21. The pharmaceutical composition of claim 20, comprising a repair template, wherein the guide RNA targets the Cas protein to the gene, resulting in Cas-mediated cleavage of the gene and insertion of the repair template.

22. The pharmaceutical composition of claim 20, wherein the polynucleotide encoding the Cas protein and the guide RNA are present in at least one viral vector.

23. The pharmaceutical composition of claim 20, wherein the polynucleotide encoding the Cas protein or the guide RNA are present in a liposome.

24. The pharmaceutical composition of claim 20, wherein the p16 gene comprises a coding sequence of SEQ ID NO: 36.

25. The pharmaceutical composition of claim 20, wherein the portion of the p16 gene comprises a single nucleotide polymorphism of an alanine residue at rs1042522.

26. The pharmaceutical composition of claim 20, wherein the guide RNA targets the Cas nuclease to a sequence of the p16 gene selected from SEQ ID NOS: 17-35.

27. The pharmaceutical composition of claim 20, wherein the Six6 gene comprises a coding sequence of SEQ ID NO: 37.

28. The pharmaceutical composition of claim 20, wherein the portion of the Six6 gene comprises single nucleotide polymorphism of a cytosine at rs33912345.

29. The pharmaceutical composition of claim 20, wherein the pharmaceutical composition is formulated as a liquid for administration with an eye dropper.

30. The pharmaceutical composition of claim 20, wherein pharmaceutical composition is formulated as a liquid for intravitreal administration.
